(19) United States
(12) Patent Application Publication
(10) Pub. No.: US 2013/0096178 A1
(43) Pub. Date: Apr. 18, 2013

(54) GENETIC MARKERS FOR PAGET'S DISEASE

(71) Applicants: Stuart H. Ralston, Edinburgh (GB); Omar Albagha, Edinburgh (GB)

(72) Inventors: Stuart H. Ralston, Edinburgh (GB); Omar Albagha, Edinburgh (GB)

(73) Assignee: THE UNIVERSITY COURT OF THE UNIVERSITY OF EDINBURGH, Edinburgh (GB)

(21) Appl. No.: 13/640,845

(22) PCT Filed: Apr. 13, 2011
(86) PCT No.: PCT/GB2011/000581

§ 371 Date: Dec. 21, 2012

(30) Foreign Application Priority Data
Apr. 14, 2010 (GB) 1006197.6

(51) Int. Cl. C12Q 1/68

(52) U.S. Cl. CPC C12Q 1/6883
USPC 514/44 A; 436/501; 435/6.12; 435/6.11; 514/44 R

(57) ABSTRACT

The present invention is based on the identification of a number of genetic markers that are associated with susceptibility to Paget's disease of bone (PDB). This invention provides details of markers and nucleotide sequences as well as associated proteins/peptides and/or compositions and methods, for use in treating, preventing and/or detecting/diagnosing PDB and/or a susceptibility/predisposition thereto.

### FIELD OF THE INVENTION

The present invention relates to genetic markers associated with Paget's disease of bone (PDB) and/or a predisposition/susceptibility thereto. Accordingly, the invention provides nucleotide sequences as well as associated proteins/peptides and/or compositions and methods, for use in preventing and/or treating PDB. The invention also extends to uses and methods related to the detection and/or diagnosis of PDB and/or a susceptibility/predisposition thereto.

### BACKGROUND OF THE INVENTION

Paget's disease of bone (PDB) is a common disorder of the skeleton that affects up to 2% of individuals of European ancestry aged 55 years and above¹. The disease is characterized by focal areas of increased and disorganized bone remodeling that can cause bone pain, bone deformity, pathological fracture, deafness and secondary osteoarthritis². Genetic factors are important contributors to PDB risk, and between 15% and 40% of individuals with PDB have an affected first-degree relative³. Mutations affecting the ubiquitin-associated domain of SQSTM1 have been identified in about 10% of individuals with what is termed ‘sporadic’ PDB and in about 40% of individuals with familial PDB^{4,5}. Despite extensive research efforts and the identification of several susceptibility loci by linkage analysis⁶⁻⁸, the remaining genes that predispose to PDB have yet to be identified.

### SUMMARY OF THE INVENTION

The present invention is based on the identification of a number of genetic markers that are associated with susceptibility to Paget's disease of bone (PDB). In particular, the inventors have determined that variations in sequence at several chromosomal loci are associated with the development of PDB and therefore provide markers for disease and/or a susceptibility/predisposition thereto.

Accordingly, this invention provides details of markers and nucleotide sequences as well as associated proteins/peptides and/or compositions and methods, for use in treating, preventing and/or detecting/diagnosing PDB and/or a susceptibility/predisposition thereto.

It should be understood that the phrase “associated with PDB disease” encompasses any link or correlation between the presence and/or absence of one or more nucleic acid sequence(s) or gene(s) and the symptoms, progression and/or development of PDB as well as a susceptibility/predisposition thereto.

One of skill will appreciate that the term “variant” as applied to the nucleic acid sequences and/or genes described herein, may encompass any variation in a nucleic acid sequence relative to, for example, a wild-type sequence or reference sequence obtained from an individual not suffering from or predisposed to PDB. Variations in a sequence may manifest as the addition, deletion, substitution and/or inversion of one or more nucleotides within a sequence. Additionally, or alternatively, the sequence variations may take the form of one or more polymorphism(s), where, for example, individual nucleotides are substituted for nucleotides not present in the wild-type or reference sequence. In other embodiments, a “variant” nucleic acid sequence may result from the occurrence of one or more mutations within the sequence. Again, as one of skill will appreciate, a mutated nucleic acid sequence may comprise one or more nucleotide inversions, additions, deletions and/or substitutions.

Each of the genetic loci identified as being associated with PDB are discussed in more detail below.

In one embodiment, the invention concerns the finding that chromosomal locus 1p13.3, is associated with PDB. Furthermore, the inventors have discovered that variations within the sequence of this region of chromosome 1 are associated with PDB. In one embodiment, the variant sequences associated with PDB are located within a 14-Kb linkage disequilibrium (LD) block, 87 Kb upstream of the CSF1 gene.

The CSF1 gene encodes macrophage colony-stimulating factor (M-CSF) which plays a critical role in osteoclast formation and survival. Accordingly, and without wishing to be bound by theory, the inventors suggest that the CSF1 gene and its product (M-CSF) are also associated with PDB. In particular, the inventors hypothesise that variations in sequences adjacent (i.e. upstream or downstream of) the CSF1 gene, for example variations within the 1p13.3 loci, may modulate the function, expression and/or activity of CSF1. In turn, this may result in a modulation of the function, expression and/or activity of the M-CSF protein which may further modulate (for example increase or decrease) osteoclast formation, differentiation and/or survival.

The variant 1p13.3 sequences provided by this invention, may comprise one or more polymorphisms. In one embodiment, the polymorphisms are selected from the group consisting of rs10494112; rs499345; and rs484959 see Table 1/Table 1 (Example 2) for details).

As such, the present invention establishes a correlation/association between variant 1p13.3 sequences (including variant CSF1 sequences) and PDB.

The invention also concerns the finding that chromosomal loci, 10p13, is associated with PDB. Furthermore, the inventors have discovered that variations within the sequence of this region of chromosome 10 are associated with PDB. In one embodiment, the variant sequences associated with PDB are located within a 30-Kb region the OPTN gene.

The OPTN gene encodes optineurin and as a result of the observations presented herein, represents a new candidate gene for PDB. Accordingly, in addition to establishing an association between 10p13 sequence variants and instances of PDB, the inventors have identified an association between modulated (for example increased and/or decreased) OPTN (or Optineurin) function, activity and/or expression and PDB.

Without wishing to be bound by theory, the inventors note that optineurin is a ubiquitously expressed cytoplasmic protein having an ubiquitin binding domain, similar to that present in the protein NEMO. Optineurin negatively regulates TNF-α-induced NF-κB activation by interacting with ubiquitylated RIP proteins. Furthermore, the inventors recognise that studies have shown that Optineurin interacts with myosin VI, suggesting it plays a role in vesicular trafficking between the Golgi apparatus and plasma membrane. Given the fact that mutations affecting the VCP protein (also involved in vesicular trafficking), cause inclusion body myopathy with early onset Paget's disease and frontotemporal dementia (IBMPFD) syndrome, modulated optineurin function, activity and/or expression, may play a role in regulating bone metabolism through effects on NF-κB signalling and vesicular trafficking.

Accordingly, variations in the sequence of the 10p13 locus, including, for example variations in the OPTN sequence and/or sequences adjacent thereto, may modulate the function, expression and/or activity of OPTN. In turn, this may result in a modulation of the function, expression and/or activity of the Optineurin protein which may further modulate (for example increase or decrease) osteoclast formation, differentiation and/or survival.

The variant OPTN sequences provided by this invention may comprise one or more polymorphisms. In one embodiment, the variant OPTN sequences comprise the polymorphism rs1561570 see Table 1/Table 1 (Example 2) for details).

In view of the above, the present invention establishes a correlation/association between sequence variations within the 10p13 locus and PDB.

The invention also concerns the finding that chromosomal locus 18q21, is associated with PDB. In particular, the inventors have discovered that variations in the sequence of this region of chromosome 18 (and in particular 18q21.33) are associated with PDB. In one embodiment, the variant sequences associated with PDB may be located near the TNFRSF11A gene and within a 22-Kb region in proximity to the TNFRSF11A gene.

TNFRSF11A encodes the receptor activator of NF-κB (RANK) which plays an important role in the differentiation and formation of osteoclasts. The inventors note that mice comprising a targeted disruption of this gene exhibit severe osteopetrosis resulting from an almost complete lack of osteoclasts. Furthermore loss-of-function mutations in TNFRSF11A cause osteoclast-poor osteopetrosis in humans. Moreover, mutations affecting the signal-peptide region of RANK cause the PDB-like syndromes of familial expansile osteoplysis, early onset familial PDB and expansile skeletal hyperphosphatasia. In view of the above, the inventors suggest that TNFRSF11A and/or RANK function, activity and/or expression, is important in the genetic regulation of bone metabolism.

Accordingly, variations in the TNFRSF11A sequence and/or sequences adjacent thereto (for example, upstream or downstream sequences) may modulate the function, expression and/or activity of TNFRSF11A. In turn, this may result in a modulation of the function, expression and/or activity of RANK which may further modulate (for example increase or decrease) osteoclast formation, differentiation and/or survival.

As such, in addition to establishing an association between 18p21 sequence variants and instances of PDB, the inventors have identified an association between modulated (for example increased and/or decreased) TNFRSF11A and/or RANK function, activity and/or expression and PDB.

The variant 18p21 sequences provided by this invention, may comprise one or more polymorphisms. In one embodiment, the polymorphisms may be selected from the group consisting of rs2957128; and rs3018362 (see (see Table 1/Table 1 (Example 2) for details).

Accordingly, the present invention establishes a correlation/association between 18p21 sequence variants and PDB.

The invention also concerns the finding that chromosomal locus 8q22.3, is associated with PDB. In particular, the inventors have discovered that variations in the sequence of this region of chromosome 8 (and in particular 8q22.3) are associated with PDB. In one embodiment, the variant sequences associated with PDB may be located in a region spanning approximately 400 kb and containing three known genes (RIMS2, TM7SF4, and DPYS). In particular, sequence variants associated with PDB cluster within an 18-kb linkage disequilibrium (LD) block spanning the entire Transmembrane 7 superfamily member 4 gene (TM7SF4).

TM7SF4 encodes dendritic cell-specific transmembrane protein (DC-STAMP) which, without wishing to be bound by theory, is a strong functional candidate gene for PDB since it is involved in osteoclast differentiation and is required for the fusion of osteoclast precursors to form mature osteoclasts. RANKL induced DC-STAMP expression is essential for osteoclast formation and connective tissue growth factor CCN2, stimulates osteoclast fusion through interaction with DC-STAMP. Since osteoclasts from patients with PDB are larger in size and contain more nuclei than normal osteoclasts, it is possible that the genetic variants that predispose to PDB at this locus (8q22.3) do so by enhancing TM7SF4 expression (causing gain-of-function).

Accordingly, variations in the TM7SF4 sequence and/or sequences adjacent thereto (for example, upstream or downstream sequences) may modulate the function, expression and/or activity of TM7SF4.

As such, in addition to establishing an association between 8q22.3 sequence variants and instances of PDB, the inventors have identified an association between modulated (for example increased and/or decreased) TM7SF4 function, activity and/or expression and PDB.

The variant 8q22.3 sequences provided by this invention, may comprise one or more polymorphisms. In one embodiment, the polymorphisms may comprise rs2458413 (see Table 1/Table 1 (Example 2) for details).

Accordingly, the present invention establishes a correlation/association between 8q22.3 sequence variants and PDB.

In one embodiment, the invention concerns the finding that chromosomal locus 7q33, is associated with PDB. Furthermore, the inventors have discovered that variations within the sequence of this region of chromosome 7 are associated with PDB. In one embodiment, the variant sequences associated with PDB are located within a ˜500 kb region containing three known genes (CNOT4, NUP205, and SLC13A4) and two predicted protein coding transcripts (PL-5283 and FAM180A).

The variant 7q33 sequences provided by this invention, may comprise one or more polymorphisms. In one embodiment, the polymorphisms comprise rs4294134, located within the 22^{nd} intron of the gene NUP205. This gene encodes a protein called nucleoporin 205 kDa which is one of the main components of the nuclear pore complex involved in the regulation of transport between the cytoplasm and nucleus⁷.

As such, the present invention establishes a correlation/association between variant 7q33 sequences (including variant NUP205 sequences) and PDB.

The invention also concerns the finding that chromosomal loci, 14q32.12, is associated with PDB. Furthermore, the inventors have discovered that variations within the sequence of this region of chromosome 14 are associated with PDB. In particular, the inventors have identified a 62 kb PDB associated region of chromosome 14 bounded by two recombination hotspots and the gene RIN3, that encodes Ras and Rab interactor 3.

Without wishing to be bound by theory, the inventors hypothesise that given the importance of small GTPases in vesicular trafficking and osteoclast function¹⁴, RIN3 may be involved in bone resorption. The inventors also note that mutations affecting the VCP, a protein also involved in vesicular trafficking, cause inclusion body myopathy with early-onset Paget's disease and frontotemporal dementia (IBMPFD)¹⁵.

Accordingly, in addition to establishing an association between 14q32.12 sequence variants and instances of PDB, the inventors have identified an association between modulated (for example increased and/or decreased) RIN3 function, activity and/or expression and PDB.

Accordingly, variations in the sequence of the 14q32.12 locus, including, for example variations in the RIN3 sequence and/or sequences adjacent thereto, may modulate the function, expression and/or activity of RIN3. In turn, this may result in a modulation of the function, expression and/or activity of the RIN3 protein which may further modulate (for example increase or decrease) osteoclast formation, differentiation and/or survival.

The variant sequences provided by this embodiment of the invention may comprise one or more polymorphisms. In one embodiment, the variant 14q32.12 sequences comprise the polymorphism rs10498635 (see Table 1/Table 1 (Example 2) for details).

In view of the above, the present invention establishes a correlation/association between sequence variations within the 14q32.12 locus and PDB.

The invention also concerns the finding that chromosomal locus 15q24.1, is associated with PDB. In particular, the inventors have discovered that variations in the sequence of this region of chromosome 15 (and in particular 15q24.1) are associated with PDB. In one embodiment, the variant sequences associated with PDB may be located in a ˜200 kb region bounded by two recombination hot spots and containing the promyelocytic leukaemia (PML) gene.

Without wishing to be bound by theory, the inventors hypothesise that the association between PDB and PML could be mediated by an effect on TGF-β signaling.

Accordingly, variations in the PML sequence and/or sequences adjacent thereto (for example, upstream or downstream sequences) may modulate the function, expression and/or activity of PML. In turn, this may result in a modulation of the function, expression and/or activity of PML which may further modulate (for example increase or decrease) osteoclast formation, differentiation and/or survival via effects on TGF-β signalling.

As such, in addition to establishing an association between 15q24.1 sequence variants and instances of PDB, the inventors have identified an association between modulated (for example increased and/or decreased) PML and/or PML function, activity and/or expression and PDB.

The variant 15q24.1 sequences provided by this invention, may comprise one or more polymorphisms. In one embodiment, the polymorphisms comprise rs5742915, which results in a phenylalanine to leucine amino acid change at codon 645 (F645L) of the PML protein (see Table 1/Table 1 (Example 2) for details).

Accordingly, the present invention establishes a correlation/association between 15q24.1 sequence variants and PDB.

The invention also concerns the finding that chromosomal loci, 6p22.3, is associated with PDB. Furthermore, the inventors have discovered that variations within the sequence of this region of chromosome 6, near the Prolactin gene PRL, are associated with PDB.

Without wishing to be bound by theory, it is suggested that Prolactin may affect bone metabolism by reducing sex hormone levels. Further, studies have shown that prolactin decreases the ratio of RANKL/OPG in human fetal osteoblast cells (Seriwatanachai D et al Cell Biol International 2008) and in animal models, prolactin was found to inhibit osteoclastic activity (Takahashi, H. et al Zoological Science, 2008)

Accordingly, in addition to establishing an association between 6p22.3 sequence variants and instances of PDB, the inventors have identified an association between modulated (for example increased and/or decreased) PRL function, activity and/or expression and PDB.

Accordingly, variations in the sequence of the 6p22.3 locus, including, for example variations in the PRL sequence and/or sequences adjacent thereto, may modulate the function, expression and/or activity of PRL. In turn, this may result in a modulation of the function, expression and/or activity of the prolactin protein which may further modulate (for example increase or decrease) bone metabolism and osteoclast activity.

The variant sequences provided by this embodiment of the invention may comprise one or more polymorphisms. In one embodiment, the variant 6p22.3 sequences comprise the polymorphism rs1341239 (see Table 1/Table 1 (Example 2) for details).

In view of the above, the present invention establishes a correlation/association between sequence variations within the 6p22.3 locus and PDB.

The invention also concerns the finding that chromosomal loci, Xq24, is associated with PDB. Furthermore, the inventors have discovered that variations within the sequence of this region of chromosome X are associated with PDB. In one embodiment, the variant sequences associated with PDB may be located within the SLC25A43 gene encoding a member of the mitochondrial carrier family of proteins.

Accordingly, in addition to establishing an association between Xq24 sequence variants and instances of PDB, the inventors have identified a possible association between modulated (for example increased and/or decreased) SLC25A43 function, activity and/or expression and PDB

Accordingly, variations in the sequence of the Xq24 locus, including, for example variations in the SLC25A43 sequence and/or sequences adjacent thereto, may modulate the function, expression and/or activity of SLC25A43. In turn, this may result in a modulation of the function, expression and/or activity of the solute carrier family 25, member 43 protein which may further modulate (for example increase or decrease) osteoclast formation, differentiation and/or survival.

The variant sequences provided by this embodiment of the invention may comprise one or more polymorphisms. In one embodiment, the variant Xq24 sequences comprise the polymorphism rs5910578 (see Table 1/Table 1 (Example 2) for details).

In view of the above, the present invention establishes a correlation/association between sequence variations within the Xq24 locus and PDB.

As stated, the invention relates to variant nucleic acid and/or gene sequences that are associated with PDB. For convenience, it is reiterated that the term “variant” relates to nucleic acid (or gene) sequences which, when compared to corresponding wild-type/reference sequences, or sequences derived from subjects not suffering from or susceptible/predisposed to PDB, comprise one or more nucleotide variations and/or mutations comprising nucleotide additions, deletions, inversions and/or substitutions.

In particular, the invention relates to the CSF1, OPTN, TNFRSF11A, TM7SF4 and RIMS2, DPYS, CNOT4, NUP205 SLC13A4, RIN3, PML, PRL and SLC25A43 genes and their products (including M-CSF, Optineurin, RANK, DC-STAMP, nucleoporin and Ras and Rab interactor 3) which have, for the first time, been associated with PDB. Hereinafter, these genes (i.e. those described in detail above) will be collectively referred to as “PDB associated genes”. Similarly, the products of each of these PDB associated genes will be referred to as “PDB associated proteins”. One of skill in this field will understand that a wild-type or reference sequence may comprise those deposited in the SNP database http://www.ncbi.nlm.nih.gov/snp as summarised in Table 1/Table 1 (Example 2), for the most strongly associated genetic markers. Additionally, or alternatively, wild-type or reference sequences for each of the genetic loci described herein may be obtained from the genome database at: http://www.ncbi.nlm.nih.gov/sites/entrez?db=Genome&itool=toolbar.

In view of the above, it should be understood that subjects in which the expression, function and/or activity of one or more PDB associated gene(s) and/or protein(s) is/are modulated, may be suffering from PDB or may be at altered risk of (i.e. susceptible or predisposed to) developing PDB.

In addition to identifying an association between certain genes, their protein products and PDB, the inventors have also identified and characterised a number of chromosomal loci linked to PDB. In particular, the inventors have determined that the loci 1p13.3, 10p13, 18q21, 8q22.3, 7q33, 14q32.12, 15q24.1, 6p22.3 and Xq24 are associated with PDB. For convenience, these chromosomal loci will be collectively referred to as “PDB associated chromosomal loci”.

In one embodiment, the inventors have established that variations in the nucleic acid sequence of these parts of chromosomes 1, 10, 18, 8, 7, 14, 15, 6 and X are associated with PDB. In some cases, these sequence variations may affect the function, expression and/or activity of the PDB associated genes provided by this invention. For example, the sequence variations may reside in regulatory sequences associated with the expression, function or activity of the PDB associated genes—such variations leading to the modulated expression, function and/or activity of PDB associated genes.

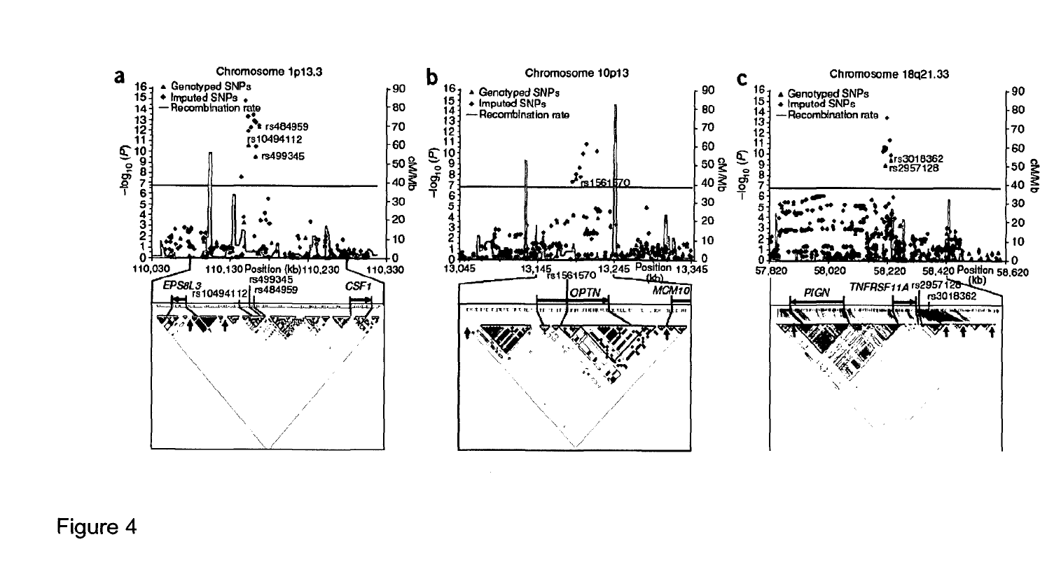

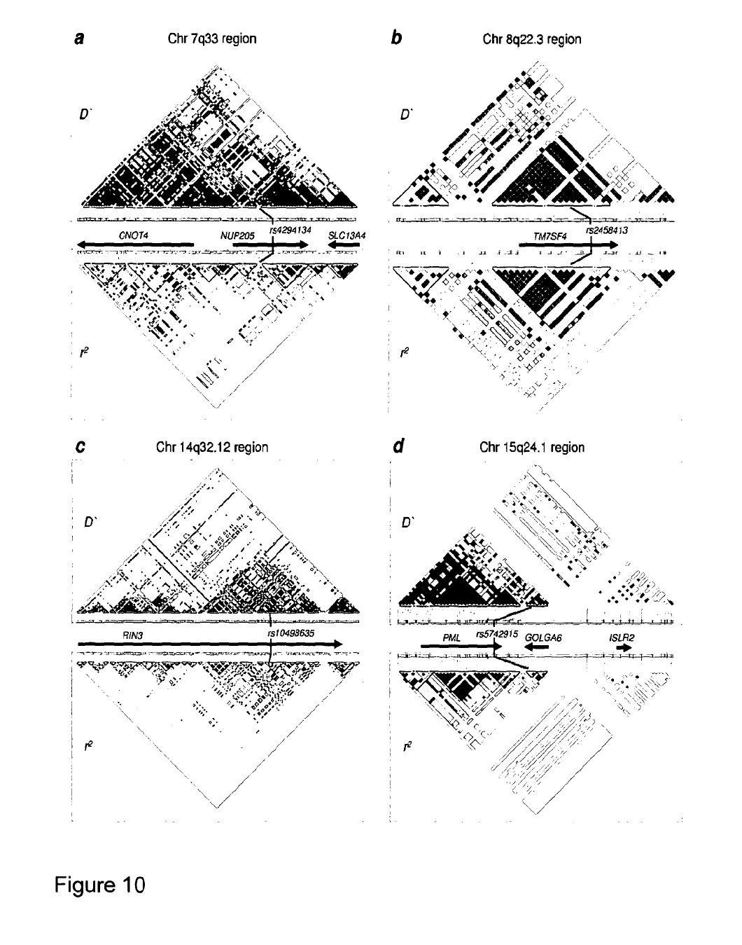

In one embodiment, the sequence variations take the form of one or more polymorphisms (i.e. SNPs) and the details of certain, specific SNPs are detailed herein (see Table 1/Table 1 (Example 2)). However, one of skill will appreciate that the invention relates to other SNPs which are themselves associated with these specific SNPs. For example, the invention also relates to SNPs which are identified as being in linkage disequilibrium (in other words SNPs which are proximal/close to and linked) with any one of the SNPs detailed in Table 1. Accordingly, the data presented in, for example, FIG. 4, FIG. 10 and Tables 3, 3 (Example 2) and 4 represents the identification of SNPs in likage disequilibrium with the specific SNPs described herein (such as those presented in Table 1/Table 1 (Example 2)).

In view of the above, a further aspect of this invention provides a method of diagnosing PDB in a subject or detecting or identifying an altered risk of developing PDB in a subject, said method comprising the step of identifying any modulation in the function, expression and/or activity of a (or one or more of) PDB associated gene/protein, in a sample provided by a subject, wherein modulated function, expression and/or activity indicates a positive diagnosis of PDB and/or an altered risk of developing PDB.

Additionally, or alternatively, the method of diagnosing PDB in a subject or detecting or identifying an altered risk of developing PDB in a subject, may comprise the step of, identifying or detecting in a sample, provided by said subject, sequence variations within one or more of the PDB associated chromosomal loci detailed above.

In one embodiment, the sequence variations may take the form of one or more SNPs at a locus selected from the group consisting of:

(i) position 110,154,000 on chromosome 1;

(ii) position 110,163,205 on chromosome 1;

(iii) position 110,167,606 on chromosome 1;

(iv) position 105,428,608 on chromosome 8;

(v) position 13,195,732 on chromosome 10;

(vi) position 58,211,715 on chromosome 18;

(vii) position 58,233,073 on chromosome 18;

(viii) position 134,943,668 on chromosome 7

(ix) position 92,173,062 on chromosome 14

(x) position 72,123,686 on chromosome 15

(xi) position 22,412,183 on chromosome 6; and

(xii) position 118,451,730 on chromosome X.

It should be noted that the nucleotide positions are based on NCBI human genome build 36 and thus, over time, these positions/co-ordinates may alter (although the “rs” notation (see below) will still apply and one of skill would easily be able to determine the new position.

In one embodiment, the variations at each of the above described loci, may comprise the addition, deletion, insertion, inversion, substitution and/or mutation of one or ore nucleotides. In a further embodiment, the variations are single nucleotide polymorphisms (SNPs).

In a further embodiment, the sequence variations within the PDB associated chromosomal loci may comprise one or more SNPs which are in linkage disequilibrium with SNPs occurring at each of the loci listed as (i)-(vii) above—examples of such SNPs being identified in FIG. 4, FIG. 10 and Tables 3, 3 (Example 2) and 4.

In a yet further embodiment, the methods described herein may comprise the step of detecting one or more of the specific SNPs selected from the group consisting of:
(i) rs10494112;
(ii) rs499345;
(iii) rs484959;
(iv) rs2458413;
(v) rs1561570;
(vi) rs2957128;
(vii) rs3018362;
(viii) rs4294134;
(ix) rs10498635;
(x) rs5742915;
(xi) rs1341239
(xii) rs5910578
(xiii) SNPs or sequence variations in linkage disequilibrium with any of (i)-(xii).

Further details regarding each of these exemplary SNPs are given in Table 1/Table 1 (Example 2).

In one embodiment, the methods of detecting or diagnosing PDB and/or a predisposition or susceptibility thereto, comprise the step of analysing the nucleotides present at each of the alleles associated with SNPs (i)-(xiii) above and determining a risk allele score. One of skill will appreciate that a risk allele score reflects the number of risk alleles present at any one allele. A risk allele score can be 0 (no risk alleles present), 1 (1 risk allele present) or 2 (2 risk alleles present). A risk allele score can be determined for one or more of the SNP alleles listed above. The total risk allele score (i.e. the sum of the score calculated for one or more of the SNPs listed above) may then be used to predict the disease risk in a patient.

In one embodiment, the method of detecting or diagnosing PDB and/or a predisposition or susceptibility thereto, comprises the step of identifying the presence or absence of risk alleles at the SNP denoted rs10494112, wherein the presence of one or more risk alleles represents a disease risk. It should be understood that the “risk allele” at this SNP is “G”.

In a further embodiment, methods for detecting or diagnosing PDB and/or a predisposition or susceptibility thereto and which comprise the step of identifying the presence or absence of risk alleles at the SNP denoted rs10494112, may further comprise the steps of identifying the presence or absence of risk alleles at one or more other SNPs selected from the group consisting of:
(i) rs499345;
(ii) rs484959;
(iii) rs2458413;
(iv) rs1561570;
(v) rs2957128;
(vi) rs3018362;
(vii) rs4294134;
(viii) rs10498635;
(ix) rs5742915;
(x) rs1341239; and
(xi) rs5910578;
(xii) SNPs or sequence variations in linkage disequilibrium with any of (i)-(xi);

wherein the presence of risk alleles at one or more of SNP locations (a)-(f) above, represents a disease risk.

It should be understood that the risk alleles at each of the above-mentioned SNP locations (i)-(xiii) are A, G, A, T, A, A, G, C, C, T and C respectively.

Based on the methods described above, the inventors have produced the following system, whereby one calculates the number of risk alleles detected in a sample provided by a subject being tested, adds the number of risk alleles for each SNP tested and uses the following table to predict disease risk in that subject, wherein the scores highlighted in grey represent an increase disease risk.

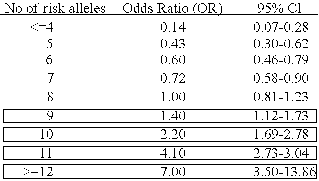

One of skill will appreciate that the results of any of the methods described herein may be compared to the results of control or reference values obtained from healthy subjects or subjects known not to be susceptible or predisposed to PDB. For example, where an assay determines the level of modulation of activity, function and/or expression of a PDB associated gene, the results may be compared to the level of expression, function and/or activity of the same PDB associated gene in a reference or control sample.

One of skill will appreciate that, in the early stages, PDB is an asymptomatic disease and thus diagnosis of early stage disease and/or identifying individuals who would benefit from early treatment (as a prophylactic, delaying or preventative measure) is difficult. Accordingly, the present invention provides methods which may be used to predict the likelihood of an individual developing PDB and recommend that individual for early treatment. Accordingly, the methods described herein may be used as a predictive test.

In addition, the methods described herein may be used in combination with existing PDB diagnostic methods—including those relying on variant SQSTM1 sequences. In this way, while existing tests may be able to predict disease (or at least an increased risk of disease) in about 10% of those who are predisposed/susceptible to PDB or who will go on to develop PDB, by combining these existing tests with the new methods described herein, it may be possible to significantly increase the number of people identified as likely to get PDB by about 80% (to about 90% of the population actually susceptible to PDB).

Detecting the modulated function, expression and/or activity of a PDB associated gene/protein and/or one or more variants the sequence of loci 1p13.3, 10p13, 18q21, 8q22.3, 7q33, 14q32.12, 15q24.1, 6p22.3 and Xq24 in a sample from a subject, indicates that that subject might have PDB or might be predisposed or susceptible to developing PDB. Accordingly, the subject to be tested may be known to be suffering from PDB or may be suspected of having PDB. In other embodiments, the subject may be healthy (i.e. with no symptoms of PDB) or may be suspected of being susceptible or predisposed to developing PDB—perhaps because of familial cases.

The methods provided by this invention may require the use of a sample comprising nucleic acid or from which nucleic acid can be obtained. The sample may be provided by (or obtained from) a subject to be tested and may take the form of a tissue biopsy, scraping or swab. Furthermore, the sample may comprise a fluid, for example a bodily fluid, such as whole blood, serum, sweat, plasma, semen, urine, lymph amniotic fluid, tissue and/or glandular secretions and/or saliva.

The Methods which may be used to detect modulated expression, function and/or activity of PDB associated gene(s), or variant nucleic acid sequences at each of the genetic loci described herein, are well known to one of skill in this field and may include, polymerase chain reaction (PCR) based techniques utilising, for example using genomic DNA as template or reverse transcriptase (RT)-PCR (see below) techniques in combination with real-time PCR (otherwise known as quantitative PCR). Other useful techniques may include restriction fragment length polymorphism analysis (RFLP) and/or hybridisation techniques using probes and/or primers designed to hybridise under conditions of high, medium and/or low stringency, to sequences within these loci. Suitable probes and/or primers (i.e. oligonucleotide sequences) are described herein (see below). Further information on such techniques may be found in Molecular Cloning: A Laboratory Manual (Third Edition) By Sambrook, MacCallum & Russell, Pub. CSHL; ISBN 978-087969577-4—incorporated herein by reference.

For example, PCR techniques useful in the detection of variant nucleic acid sequences may require the use of short oligonucleotide primers designed to hybridise to sequences proximal to (for example 3′ and 5′ (upstream or downstream)) of a nucleic acid sequence of interest—for example a sequence potentially harbouring a variant sequence. Once oligonucleotides have been hybridised to a nucleic acid sequence, the nucleic acid sequence between the primers is enzymatically amplified via the PCR. The amplified nucleic acid may then be sequenced to determine whether or not it comprises a variant sequence. Additionally, or alternatively, the amplified nucleic acid may be contacted with one or more restriction enzymes—this technique is particularly useful if a variant nucleic acid sequence is known either to remove a particular restriction site or create a restriction site. The presence or absence of a variant nucleic acid sequence may be detected via analysis of the resulting restriction fragment length polymorphism (RFLP) profile. When analysing RFLP profiles, the results may be compared to standard or control profiles obtained by contacting nucleic acid sequences obtained from health patients (i.e. patients not suffering from or predisposed to PDB), with the same restriction enzymes.

In addition to the above, altered electophoretic mobility may be used to detect alterations in nucleic acid sequences. For example, small sequence deletions and insertions may be visualised by high resolution gel electrophoresis—nucleic acid sequences with different sequences migrating through agarose gels (denaturing or non-denaturing and/or gradient gels) at different speeds/rates.

One of skill will appreciate that relative levels of mRNA expression may used as a means of determining the level of expression, activity and/or function of a particular gene (such as, for example, the PDB associated genes described herein). By way of example, modulation (i.e. an increase or decrease) in the amount of mRNA encoding the genes now found to be associated with PDB, may indicate modulated gene expression, function and/or activity and may further indicate that a subject is suffering from or predisposed/susceptible to, PDB. More specifically, real time-PCR may used to determine the level of expression of any of the PDB associated genes described herein. Typically, and in order to quantify the level of expression of a particular nucleic acid sequence, RT-PCR may be used to reverse transcribe the relevant mRNA to complementary DNA (cDNA). Preferably, the reverse transcriptase protocol may use primers designed to specifically amplify an mRNA sequence of interest (in this case mRNA encoding all or part of a PDB associated gene). Thereafter, PCR may be used to amplify the cDNA generated by reverse transcription. Typically, the cDNA is amplified using primers designed to specifically hybridise with a certain sequence and the nucleotides used for PCR may be labelled with fluorescent or radiolabelled compounds.

One of skill in the art will be familiar with the technique of using labelled nucleotides to allow quantification of the amount of DNA produced during a PCR. Briefly, and by way of example, the amount of labelled amplified nucleic acid may be determined by monitoring the amount of incorporated labelled nucleotide during the cycling of the PCR.

Further information regarding the PCR based techniques described herein may be found in, for example, PCR Primer: A Laboratory Manual, Second Edition Edited by Carl W. Dieffenbach & Gabriela S. Dveksler: Cold Spring Harbour Laboratory Press and Molecular Cloning: A Laboratory Manual by Joseph Sambrook & David Russell: Cold Spring Harbour Laboratory Press.

Other techniques that may be used to determine the level of PDB associated gene expression in a sample, include, for example, northern and/or Southern blot techniques. A northern blot may be used to determine the amount of a particular mRNA present in a sample and as such, could be used to determine the amount of PDB associated gene expression. Briefly, total or messenger (m)RNA may be extracted from any of the samples described above using techniques known to the skilled artisan. The extracted RNA may then be subjected to electrophoresis. A nucleic acid probe, designed to hybridise (i.e. complementary to) an RNA sequence of interest—in this case the mRNA encoding all or part of a PDB associated gene, may then be used to detect and quantify the amount of a particular mRNA present in a sample.

Additionally, or alternatively, a level of PDB associated gene expression may be identified by way of microarray analysis. Such a method would involve the use of a DNA micro-array which comprises nucleic acid derived from PDB associated genes. To identify a level of PDB associated gene expression, one of skill in the art may extract the nucleic acid, preferably the mRNA, from a sample and subject it to an amplification protocol such as, RT-PCR to generate cDNA. Preferably, primers specific for a certain mRNA sequence—in this case sequences encoding PDB associated genes may be used.

The amplified PDB associated gene cDNA may be subjected to a further amplification step, optionally in the presence of labelled nucleotides (as described above). Thereafter, the optionally labelled amplified cDNA may be contacted with the microarray under conditions which permit binding with the DNA of the microarray. In this way, it may be possible to identify a level of PDB associated gene expression.

In addition, other techniques such as deep sequencing and/or pyrosequencing may be used to detect PDB associated sequences and/or variant 1p13.3, 10p13, 18q21, 8q22.3, 7q33, 14q32.12, 15q24.1, 6p22.3 and Xq24 sequences, in any of the samples described above. Further information on these techniques may be found in “Applications of next-generation sequencing technologies in functional genomics”, Olena Morozovaa and Marco A. Marra, Genomics Volume 92, Issue 5, November 2008, Pages 255-264 and “Pyrosequencing sheds light on DNA sequencing”, Ronaghi, Genome Research, Vol. 11, 2001, pages 3-11.

In other embodiments, samples provided by subjects to be tested may be analysed or probed for the levels of each of the PDB associated proteins described herein. For example, immunological detection techniques such as, for example, enzyme linked immunosorbent assays (ELISAs) or Western blot and/or immunoblot techniques may be used. Such techniques may require the use of binding agents or antibodies specific to, or selective for, the various PDB associated gene products (or fragments thereof) described herein. Further information on such techniques may be found in Using Antibodies: A Laboratory Manual By Harlow & Lane, Pub. CSHL, ISBN 978-087969544-6 and Antibodies: A Laboratory Manual by Harlow & Lane, CSHL, ISBN 978-087969314-5—both of which are incorporated herein by reference.

In one embodiment, binding agents (for example antibodies) having affinity/specificity/selectivity to/for any of the PDB associated proteins (or epitopes thereof), may be coated onto the surface of a suitable substrate (for example a microtitre plate). Thereafter, the coated substrate may be contacted with a sample to be tested for the presence or absence of PDB associated proteins. Binding between any PDB associated proteins present in the sample and the binding agents coated onto the surface of the substrate, may be detected by means of a secondary binding agent having specificity for a PDB associated protein. Secondary antibodies useful in the present invention may optionally be conjugated to moieties which permit them to be detected (referred to hereinafter as “detectable moieties”). For example, the secondary antibodies may be conjugated to an enzyme capable of reporting a level via a colourmetric chemiluminescent reaction. Such conjugated enzymes may include but are not limited to Horse Radish Peroxidase (HRP) and Alkaline Phosphatase (AlkP). Additionally, or alternatively, the secondary antibodies may be conjugated to a fluorescent molecule such as, for example a fluorophore, such as FITC, rhodamine or Texas Red. Other types of molecule which may be conjugated to binding agents include radiolabelled moieties.

Other techniques which exploit the use of agents capable of binding PDB associated proteins, for example antibodies, include, for example, techniques such as western blot or dot blot. A western blot may involve subjecting a sample to electrophoresis so as to separate or resolve the components, for example the proteinaceous components, of the sample. The resolved components/proteins may then be transferred to a substrate, such as nitrocellulose.

In order to identify any PDB associated proteins present in a sample, the substrate (for example nitrocellulose substrate) to which the resolved components and/or proteins have been transferred, may be contacted with a binding agent capable of binding PDB associated proteins under conditions which permit binding between any PDB associated proteins present in the sample (or transferred to the substrate) and the agents capable of binding the PDB associated proteins.

Advantageously, the agents capable of binding the PDB associated proteins may be conjugated to a detectable moiety.

Additionally, the substrate may be contacted with a further binding agent having affinity for the binding agent(s) capable of binding PDB associated proteins. Advantageously, the further binding agent may be conjugated to a detectable moiety.

In certain embodiments any of the samples described above may be used a source of PDB associated protein. Additionally or alternatively, the PDB associated protein may be isolated or purified from the sample, or produced in recombinant form.

Other immunological techniques which may be used to identify a level of PDB associated protein in a sample (particularly tissue or biopsy samples) include, for example, immunohistochemistry wherein PDB associated protein binding agents, are contacted with a sample such as those described above, under conditions which permit binding between any PDB associated protein present in the sample and the binding agent. Typically, prior to contacting the sample with the binding agent, the sample is treated with, for example a detergent such as Triton X100. Such a technique may be referred to as “direct” immunohistochemical staining.

Alternatively, the sample to be tested may be subjected to an indirect immunohistochemical staining protocol wherein, after the sample has been contacted with a PDB associated protein binding agent, a further binding agent (a secondary binding agent) which is specific for, has affinity for, or is capable of binding the PDB associated protein antigen binding agent, is used to detect PDB associated protein/binding agent complexes.

The skilled person will understand that in both direct and indirect immunohistochemical techniques, the binding agent or secondary binding agent may be conjugated to a detectable moiety. Preferably, the binding agent or secondary binding agent is conjugated to a moiety capable of reporting a level of bound binding agent or secondary binding agent, via a colourmetric chemiluminescent reaction.

In order to identify the levels of PDB associated protein present in a sample, one may compare the results of any of the immunological of molecular (i.e. PCR, RT-PCR) techniques described herein, with results obtained from the same procedures using control or reference samples obtained from subjects not suffering from PDB and/or not predisposed or susceptible thereto. By way of example, a sample revealing an increased or decreased level of PDB associated gene activity, expression or function and/or an increased or decreased level of PDB associated protein than detected in a corresponding reference or control sample, may have been provided by a subject with PDB or susceptible or predisposed thereto.

In a further aspect the present invention, provides compounds for example polynucleotides and/or polypeptides (proteins, peptides or amino acids), useful in the treatment or prevention of PDB.

Accordingly, in one embodiment, the present invention relates to polynucleotide and/or polypeptide fragments for use in treating or preventing PDB.

In a further aspect, the invention relates to the use of polynucleotide and/or polypeptide fragments for the manufacture of a medicament for treating or preventing PDB.

In a yet further aspect, the present invention provides a method of treating or preventing PDB, said method comprising the step of administering to a subject in need thereof, a therapeutically effective amount of a polynucleotide and/or polypeptide compound.

One of skill will appreciate that a “polynucleotide” compound comprises a chain of DNA or RNA nucleotides—also known as an oligonucleotide. Where the polynucleotide sequences find application in the treatment or prevention of PDB, they should be designed to restore wild-type gene expression, function and/or activity and/or to correct any aberrant (i.e. increased or decreased) gene expression, function and/or activity.

The methods, uses, medicaments and/or compositions provided by this invention may comprise polynucleotides and/or polypeptides, wherein said polynucleotides and/or polypeptides modulate the expression, activity and/or function of the CSF1, OPTN, TNFRSF11A, TM7SF4, RIMS2, DPYS, CNOT4, NUP205 SLC13A4, RIN3, PML, PRL and/or SLC25A43 genes described herein.

By way of example, the polynucleotide compounds of the present invention may comprise all or part of the sequence of the PDB associated genes described herein. Such sequences may be used in gene therapy techniques to restore wild-type PDB associated gene expression, function and/or activity in subjects suffering from PDB or susceptible/predisposed thereto. Accordingly, the polynucleotide sequences for use in the treatment or prevention of PDB may comprise sequences derived from wild-type, or normally functioning, PDB associated genes.

In one embodiment, the polynucleotide sequences for use in the compositions and medicaments of this invention, comprise the CSF1, OPTN, TNFRSF11A, TM7SF4, RIMS2, DPYS, CNOT4, NUP205 SLC13A4, RIN3, PML, PRL and/or SLC25A43 gene sequences or fragments or portions thereof. Accordingly, the present invention provides polynucleotide sequences derived from the CSF1, OPTN, TNFRSF11A, TM7SF4, RIMS2, DPYS, CNOT4, NUP205 SLC13A4, RIN3, PML, PRL and/or SLC25A43 genes, for use in treating and/or preventing PDB. Compositions comprising polynucleotide sequences provided by this invention may be administered to subjects suffering from PDB or to those who are at risk of developing PDB.

In other embodiments, polynucleotide sequences of this invention may comprise antisense sequences which may be used to, for example, suppress aberrant gene expression. Where, for example, one or more of the genes CSF1, OPTN, TNFRSF11A, TM7SF4, RIMS2, DPYS, CNOT4, NUP205 SLC13A4, RIN3, PML, PRL and/or SLC25A43 is aberrantly expressed due to, for example, a variation in the gene sequence itself or some other sequence within the chromosomal loci described herein, an antisense oligonucleotide may be used to modulate, preferably suppress or ablate, the aberrant gene expression. Antisense oligonucleotides may comprise DNA and/or RNA. In the case of RNA based antisense oligonucleotide sequences, the oligonucleotide may take the form of a small/short interfering and/or silencing RNA—such molecules being referred to hereinafter as siRNA. One of skill will appreciate that RNA molecules of this type may be modified in some way so as to be nuclease resistant.

By analysing the sequence of the various loci of this invention, one of skill may utilise algorithms such as, for example, BIOPREDsi, to determine or computationally predict nucleic acid sequences that have an optimal knockdown effect for a particular gene sequence. Accordingly, the skilled person may easily and without burden, prepare and test a library of different oligonucleotides to determine whether or not any are capable of modulating the expression, function and/or activity of any of the CSF1, OPTN, TNFRSF11A, TM7SF4, RIMS2, DPYS, CNOT4, NUP205 SLC13A4, RIN3, PML, PRL and/or SLC25A43 genes.

Polypeptide sequences provide by this invention may take the form of proteins, polypeptides or amino acids, comprising sequences derived from or comprising the PDB associated genes described herein. For example, the medicaments, uses, methods and/or compositions provided by this invention may comprise polypeptides designed to modulate or mimic the expression, function or activity of a PDB associated protein. More specifically, where the expression, activity or function of a PDB associated protein is aberrant resulting in PDB or a susceptibility/predisposition thereto, polypeptides comprising wild-type or normal PDB associated protein sequences may be used to treat or prevent PDB.

One of skill will readily understand that genes homologous to the human PDB associated genes provided by this invention, may be found in a number of different species, including, for example, other mammalian (particularly rodent) species.

Homologous genes may exhibit as little as approximately 20 or 30% sequence homology or identity however, in other cases, homologous genes may exhibit at least 40, 50, 60, 65 70, 75, 80, 85, 90, 91, 92, 93, 94, 95, 96, 97, 98, 99% homology to the various nucleotide sequences given above. As such, homologous genes from other species are to be included within the scope of this invention.

Furthermore, using the various nucleic acid and amino acid sequences of the genes/proteins described herein, one of skill in the art could readily identify related sequences in other species, such as other mammals etc. For example, nucleic acid obtained from a particular species may be analysed using any of the probes described herein (see below), for homologous, identical or closely related sequences.

One of skill in this field will readily understand that for the various nucleic acid sequences and polypeptides described herein, natural variations due to, for example, polymorphisms, may exist between genes and proteins isolated from any given species. These variants may manifest as proteins and/or genes that exhibit one or more amino acid/nucleic acid substitutions, additions, deletions and/or inversions relative to a reference sequence (for example any of the sequences described above). As such, it is to be understood that all such variants, especially those that are functional or display the desired activity, are to be included within the scope of this invention.

In other embodiments, the invention relates to derivatives of any of the sequences described herein. The term “derivatives” may encompass gene or peptide sequences which, relative to those described herein, comprise one or more amino acid substitutions, deletions, additions and/or inversions.

Additionally, or alternatively, analogues of the various peptides described herein may be produced by introducing one or more conservative amino acid substitutions into the primary sequence. One of skill in this field will understand that the term “conservative substitution” is intended to embrace the act of replacing one or more amino acids of a protein or peptide with an alternate amino acid with similar properties and which does not substantially alter the physcio-chemical properties and/or structure or function of the native (or wild type) protein. Analogues of this type are also encompassed with the scope of this invention.

As is well known in the art, the degeneracy of the genetic code permits substitution of one or more bases in a codon without changing the primary amino acid sequence. Consequently, although the sequences described in this application are known to encode certain proteins (each of which is described herein), the degeneracy of the code may be exploited to yield variant nucleic acid sequences which encode the same primary amino acid sequences.

The present invention also provides polynucleotide sequences comprising nucleotides which are complementary to nucleotide sequences (preferably contiguous sequences) adjacent to and/or comprising, the variant sequences described herein. In one embodiment, the polynucleotide sequences are primer or probe sequences which may otherwise be referred to as oligonucleotides. In one embodiment, the probe or primer oligonucleotides provided by this invention may comprise, for example 5-50, 6-40, 7-30, 8-20 nucleotides. In one embodiment, and particularly where the oligonucleotide has application as a probe, the oligonucleotide may comprise a nucleotide complementary to a SNP (i.e. complementary to the minor allele). One of skill will appreciate that primer sequences comprising nucleotides complementary to sequences upstream and/or downstream of any of the SNPs described herein, may be used in PCR based techniques to amplify sections of nucleic acid comprising one or more SNP or SNP loci. Oligonucleotide sequences useful as probes or primers may be used to detect the presence or absence of certain SNP sequences in nucleic acid samples provided by subjects.

In a further aspect, the present invention provides a method of treating and/or preventing PDB, said method comprising the step of obtaining a sample of nucleic acid (or a sample from which nucleic acid may be extracted or prepared) and subjecting the sample to the methods of detecting the presence or absence of a gene and/or polymorphism associated with PDB described herein, wherein subjects identified as suffering from or predisposed/susceptible to PDB are administered a medicament or composition to treat and/or prevent PDB. The medicament and/or composition may comprise any of the polynucleotides and/or polypeptides provided by this invention.

The polynucleotide and/or polypeptide sequences described herein may be isolated in that they are substantially free of any other biological material. Furthermore, the invention relates to recombinant polypeptide sequences generated using vectors comprising, for example, the CSF1, OPTN, TNFRSF11A, TM7SF4, RIMS2, DPYS, CNOT4, NUP205 SLC13A4, RIN3, PML, PRL and/or SLC25A43 genes (or fragments or portions thereof) and host cells, into which the vectors are introduced.

In one embodiment, the invention provides vectors, for example, natural or synthetic vectors, adapted to receive and, in some cases express, genes or gene fragments. Such vectors may include plasmids or expression cassettes. As such, the vectors encompassed by this invention may include plasmid constructs comprising any of the polynucleotide sequences, including the antisense oligonucleotide sequences, described herein.

Vectors of the type described above may be introduced into a suitable cell for the generation of a recombinant product of any of the PDB associated genes (or fragment thereof) described herein. Accordingly, the present invention also extends to host cells modified to comprise any of the vectors described herein. Again, further information relating to the use of cell transformation protocols (for example heat-shock, electroporation and/or chemical transformation) may be found in Molecular Cloning: A Laboratory Manual (Third Edition) By Sambrook, MacCallum & Russell, Pub. CSHL; ISBN 978-087969577-4—incorporated herein by reference.

A further aspect of this invention relates to methods of identifying compounds which modulate the expression/function and/or activity of PDB associated genes/proteins—such compounds being of use in the treatment and prevention of PDB. In one embodiment, such a method may comprise the steps of:

(a) contacting a PDB associated gene/protein with an agent to be tested; and

(b) identifying or detecting any modulation in the expression, function and/or activity of the PDB associated gene/protein.

The step of contacting a PDB associated gene/protein with a test agent, may comprise the use of a cell, for example a mammalian cell, expressing (either naturally or through recombinant manipulation) a PDB associated gene/protein. The techniques which may be used to detect modulated PDB associated gene/protein expression are discussed in detail above.

The method provided by this aspect of the invention may easily be adapted to provide micro-array or high throughput assays capable of analysing large numbers of agents for modulatory activity on the expression, function or activity of a PDB associated gene/protein.

In a further aspect, the invention provides antibodies having affinity for or selective/specific for a polypeptide (or an epitope thereof) encoded by a variant 1p13.3, 10p13, 18q21, 8q22.3, 7q33, 14q32.12, 15q24.1, 6p22.3 and Xq24 sequence. Polyclonal and/or monoclonal antibodies are easily produced and/or purifies using routine laboratory techniques. It should be understood that the term antibody also includes epitope binding fragments or derivatives such as, for example, single chain antibodies, diabodies, triabodies, minibodies and/or single domain antibodies. The term antibodies also encompasses Fab, (Fab)₂ and/or other epitope binding fragments.

Accordingly, any of the polynucleotides or polypeptides, antibodies or test agents subjected found to be potentially useful in the treatment or prevention of PDB, may be formulated as sterile pharmaceutical compositions comprising a pharmaceutically acceptable carrier or excipient. Such carriers or excipients are well known to one of skill in the art and may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycon, sodium carboxymethylcellulose, polyacrylates, waxes, polyethylene-polypropylene-block polymers, polyethylene glycol and wool fat and the like, or combinations thereof.

A further aspect of the invention relates to kits for identifying and/or determining whether or not a variant PDB associated gene/protein and/or 1p13.3, 10p13, 18q21, 8q22.3, 7q33, 14q32.12, 15q24.1, 6p22.3 and Xq24 sequence is present or absent in, for example, a nucleic acid sample. A kit according to this aspect of the invention may include one or more pairs of oligonucleotide primers useful for amplifying a nucleotide sequence of interest. For example, the nucleotide sequence of interest may comprise one or more nucleic acid sites known to harbour variant 1p13.3, 10p13, 18q21, 8q22.3, 7q33, 14q32.12, 15q24.1, 6p22.3 and Xq24 sequence associated with PDB. The kit may comprises a polymerizing agent, for example, a thermo-stable nucleic acid polymerase such as one disclosed in U.S. Pat. No. 4,889,818 or 6,077,664. Furthermore, the kit may comprises an elongation oligonucleotide that hybridizes to sequence adjacent or proximal to a site potentially harbouring a variant 1p13.3, 10p13, 18q21, 8q22.3, 7q33, 14q32.12, 15q24.1, 6p22.3 and Xq24 sequence. Where the kit includes an elongation oligonucleotide, it may also include chain elongating nucleotides, such as dATP, dTTP, dGTP, dCTP, and dITP and/or analogs thereof. In addition, the kit provided by this aspect of this invention may optionally include terminating nucleotides such as, for example, ddATP, ddTTP, ddGTP, ddCTP. The kit may include one or more oligonucleotide primer pairs, a polymerizing agent, chain elongating nucleotides, at least one elongation oligonucleotide, and one or more chain terminating nucleotides. Kits may also optionally include reaction and/or storage buffers, vials or other storage/reaction vessels, microtiter plates and instructions for use.

### DETAILED DESCRIPTION

The present invention will now be described in detail with reference to the following Figures which show:

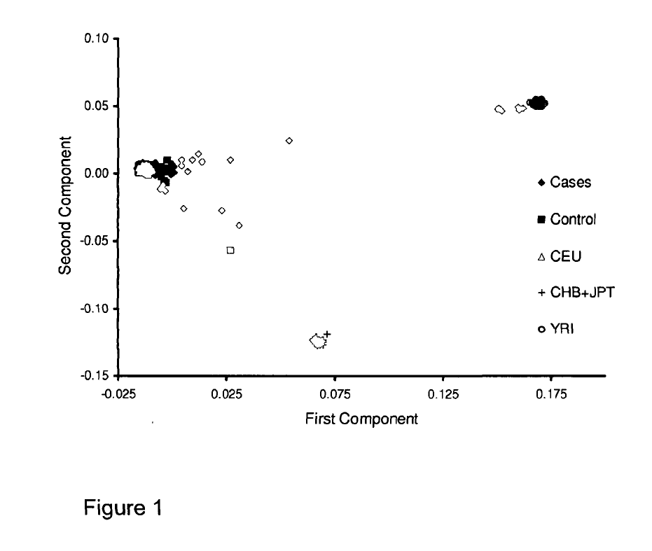

FIG. 1: The first two component of multidimensional scaling analysis of IBS sharing matrix of all case (red diamonds) and control (blue squares) samples including HapMap project samples of European (CEU; green triangles), Asian (CHB+JPT; cyan plus sign), and African (YRI; purple circles) population. Samples located outside the main CEU cluster (greyed, n=22) were excluded before testing for association with Paget's disease of bone.

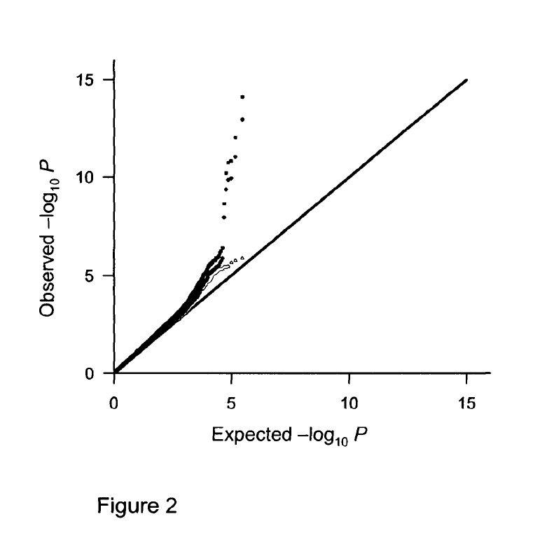

FIG. 2: A Q-Q plot showing the distribution of expected compared to observed −log₁₀ P for the association test results using stratified Cochran-Mantel-Haenszel test before (red squares; λ=1.096) and after genomic control adjustment (blue diamonds; λ=1.0004). The green triangles represent the distribution of −log₁₀ P after removal of genome wide significant and correlated SNPs from chr1, chr10, and chr18.

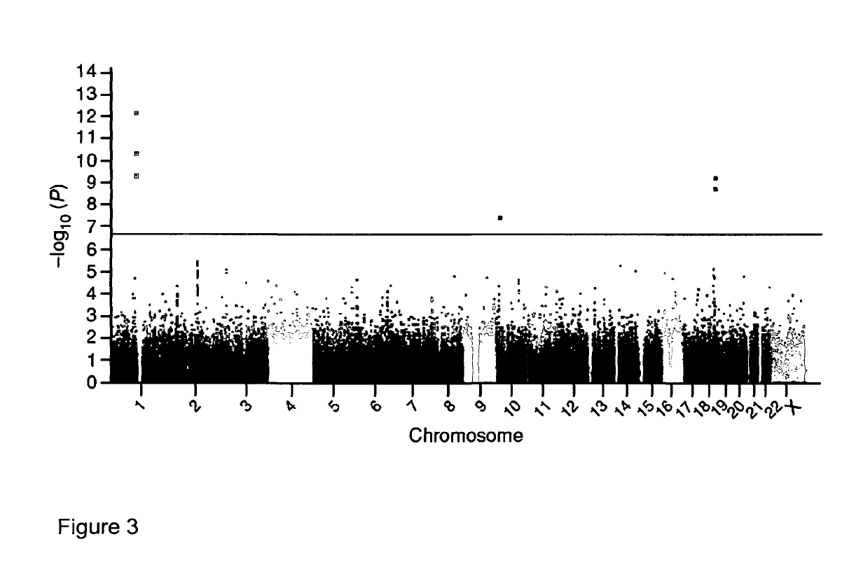

FIG. 3: Detection of loci conferring susceptibility to PDB by genome-wide association. Manhattan plot of association test results from the discovery cohort showing chromosomal positions of the 294,633 SNPs passing quality control plotted against genomic control-adjusted −log₁₀ P. Association with PDB was tested using stratified CMH tests. The red horizontal line indicates the threshold for genome-wide significance (P<1.7×10⁻⁷).

FIG. 4: Details of loci associated with PDB. (a-c) Association and LD plots of regions showing genome-wide significant association with PDB located on (a) 1p13, (b) 10p13 and (c) 18q21. The chromosomal positions (based on NCBI human genome Build 36) of the SNPs are plotted against genomic control-adjusted −log₁₀ P. Genotyped SNPs are shown as red triangles and imputed SNPs are blue diamonds. The estimated recombination rates (cM/Mb) from HapMap CEU release 22 are shown as gray lines; the red horizontal line indicates the genome-wide significance threshold (P<1.7×10⁻⁷). Genotyped SNPs were tested using stratified CMH tests; imputed SNPs were tested using a regression analysis based on imputed allelic dosage and adjusting for population clusters. SNPs reaching genome-wide significance are indicated with red text. LD plots for the indicated regions are based on HapMap CEU release 22 showing LD blocks depicted for alleles with MAF >0.05 using the r² coloring scheme of Haploview³⁷. The blue arrows indicate known genes in the region; possible recombination hot spots (>20 cM/Mb) are shown as green arrows on the LD plots.

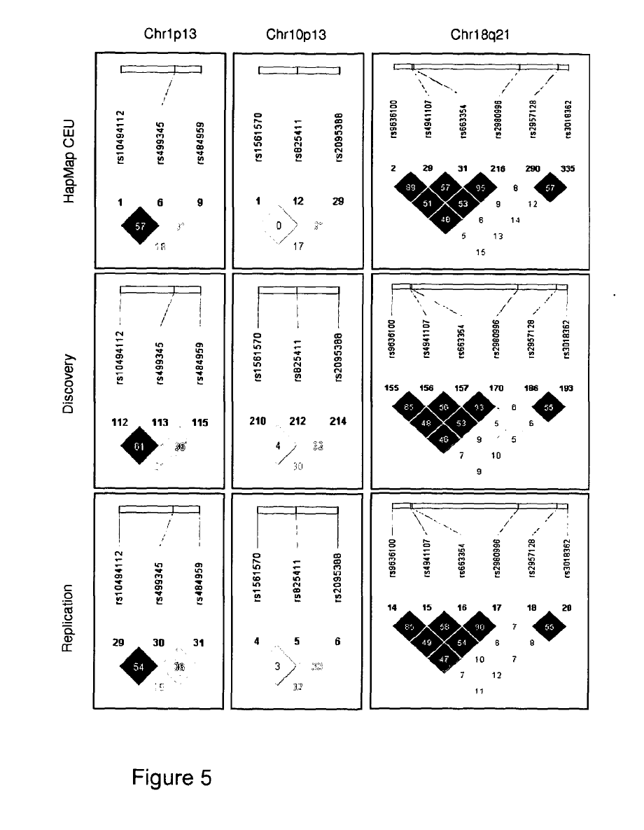

FIG. 5: Linkage disequilibrium between SNP at each of the three candidate loci are shown as depicted in the Hapmap CEU panel (top panel), the discovery sample (middle panel), and replication sample (bottom panel). LD values shown are r² determined using Haploview v 4.1.

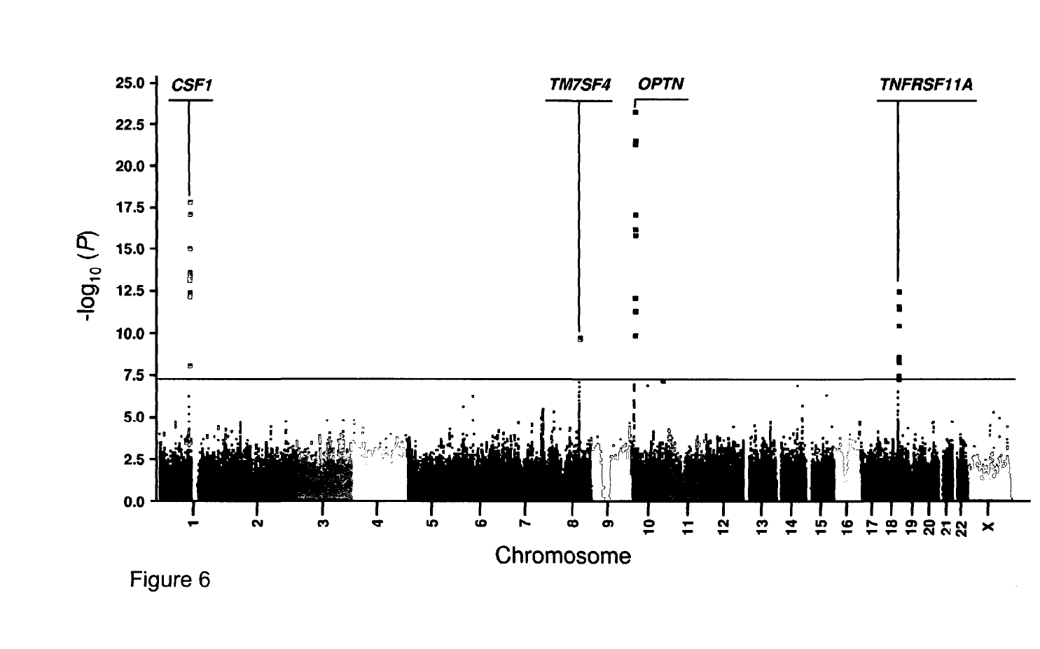

FIG. 6: Loci for susceptibility to PDB detected by genome wide association study.

Manhattan plot of association test results of GWAS stage data showing chromosomal position of 2,487,078 genotyped or imputed SNPs plotted against genomic-control adjusted −log₁₀ P. The red horizontal line represents the threshold for genome wide significance (P<5×10⁻⁸).

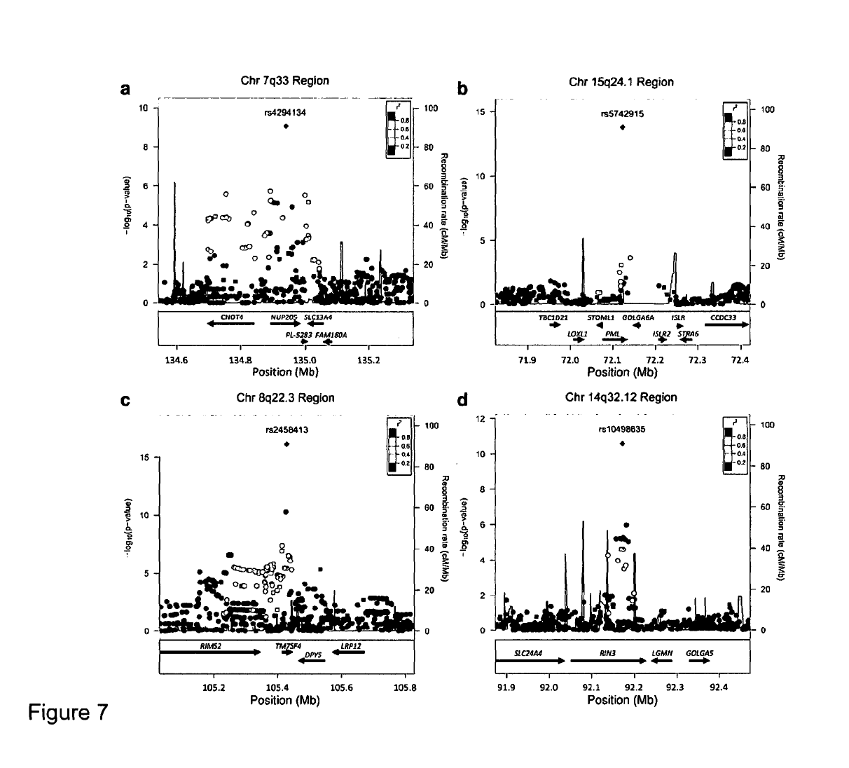

FIG. 7: Regional association plots of loci showing genome wide significant association with PDB. Details of loci on chromosome (a) 7q33, (b) 15q24.1, (c) 8q22.3 and (d) 14q32.12 showing the chromosomal position (based on NCBI human genome build 36) of SNPs in each region plotted against −log₁₀ P values. Genotyped (squares) and imputed (circles) SNPs are colour-coded according to the extent of linkage disequilibrium with the SNP showing the highest association signal (represented as purple diamonds) from each region in the combined analysis The estimated recombination rates (cM/Mb) from HapMap CEU release 22 are shown as light blue lines and blue arrows represent known genes in each region. The associated regions were defined based on LD with the highest association signal (r²>0.2) within a window of 500 kb.

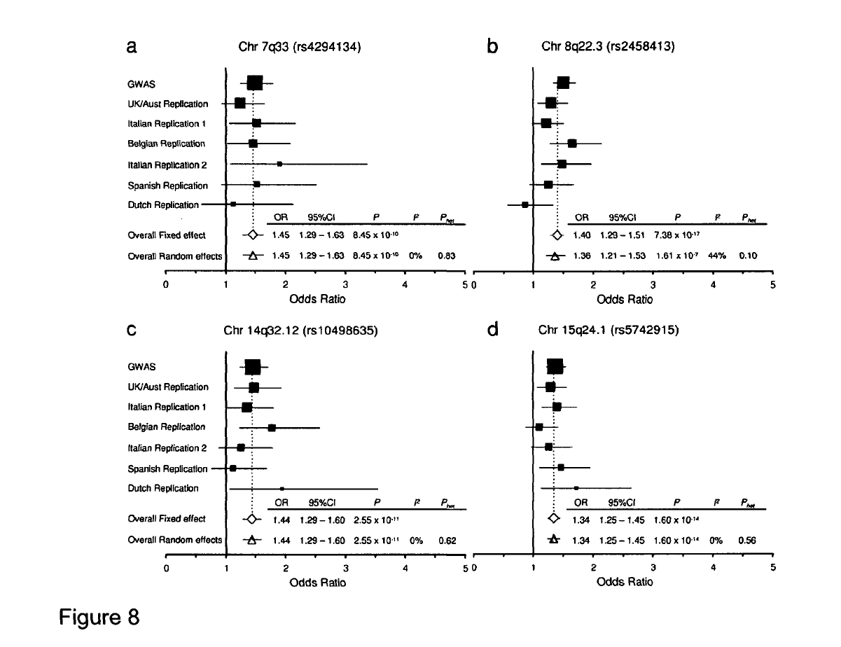

FIG. 8: Forest plots of overall effect size for SNPs associated with PDB risk from the identified loci on (a) 7q33 (rs4294134), (b) 8q22.3 (rs2458413), (c) 14q32.12 (rs10498635), and (d) 15q24.1 (rs5742915). The overall effect size was estimated using meta-analysis of the GWAS sample and the six replication samples. The black squares represent the effect estimates for the individual cohorts and the horizontal lines represent the 95% confidence interval of the estimates. The sizes of the squares are proportionate to the weight of the estimate. The diamonds and triangles represent the overall estimate under fixed effect and random effects model, respectively. Odds ratio (OR) and their 95% confidence interval (CI), P-values, I² statistics, and P-values for heterogeneity (Pₕₑₜ) are shown next to the overall effect estimate. The dotted vertical lines represent the overall fixed effect estimates.

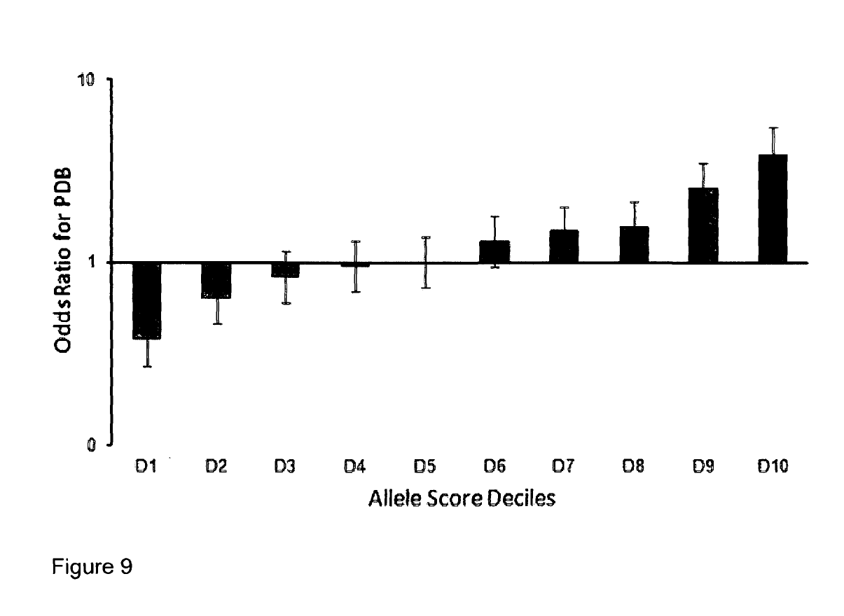

FIG. 9: Cumulative contribution of genome-wide significant loci to the risk of PDB. Risk allele scores defined by the seven loci associated with PDB risk is plotted against the odds ratio (OR) for PDB. Risk alleles were weighted according to their estimated effect size and weighted risk allele scores were divided into ten equal parts (deciles) using data from the replication cohorts. The OR for PDB risk was calculated for each decile in reference to the fifth decile (D5). Vertical bars represent 95% confidence intervals.

FIG. 10: Linkage disequilibrium patterns of newly identified or confirmed loci associated with PDB risk. Linkage disequilibrium (LD) patterns of genomic regions associated with PDB risk on (a) 7q33; (b) 8q22.3; (c) 14q32.12; and (d) 15q24.1. LD patterns were derived from the CEU HapMap release 22 data using haploview 4.2. Blue arrows represent genes in the region and the SNP showing the lowest P-values for association with PDB are shown in red text.

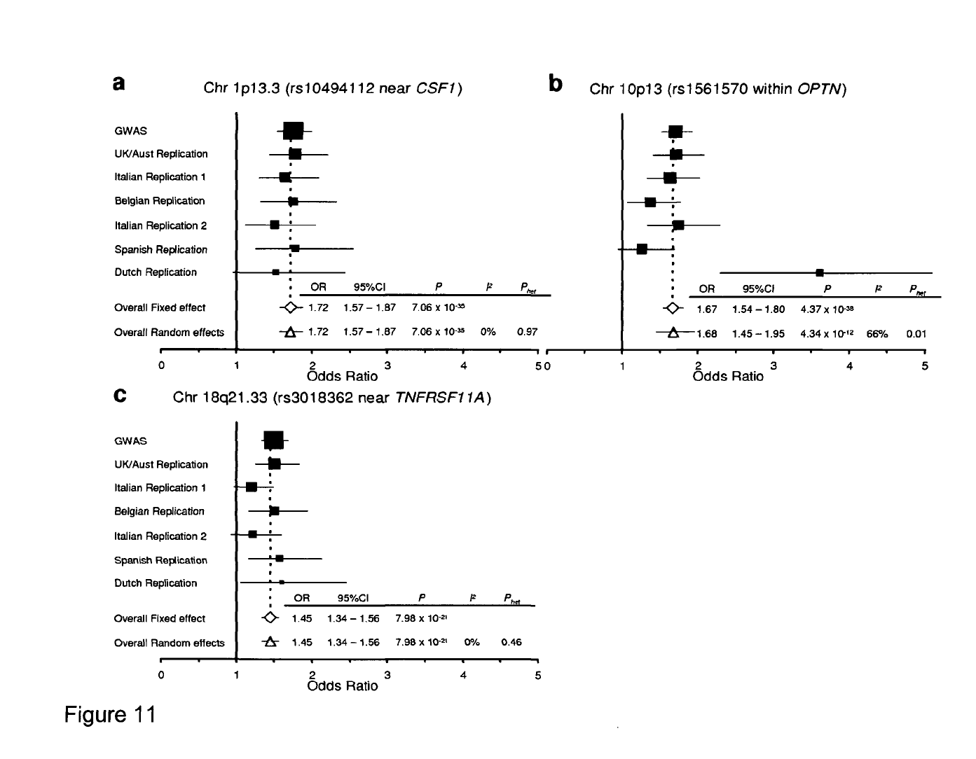

FIG. 11: Replication of association for the previously identified loci at CSF1, OPTN and TNFRSF11A. Forest plots of overall effect size for SNPs associated with PDB risk from the previously identified loci on (a) 1p13.3 (rs10494112), (b) 10p13 (rs1561570), and (c) 18q21.33 (rs3018362). The overall effect size was estimated by meta-analysis of the GWAS sample and the six replication cohorts. The black squares represent the effect estimates for the individual cohorts and the horizontal lines represent the 95% confidence interval of the estimates. The sizes of the squares are proportionate to the weight of the estimate for each cohort. The diamonds and triangles represent the overall estimates under fixed effect and random effects models, respectively. Odds ratio (OR) and their 95% confidence interval (CI), P-values, I² statistics, and P-values for heterogeneity (Pₕₑₜ) are shown next to the overall effect estimate. The dotted vertical lines represent the overall fixed effect estimates.

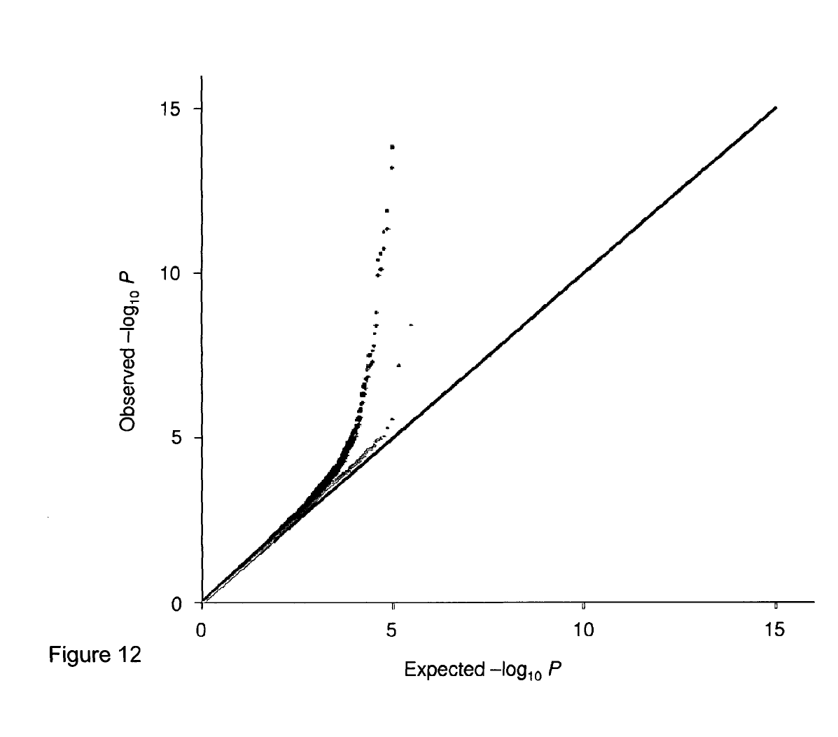

FIG. 12: Quantile-Quantile plot of association test results A Q-Q plot showing the distribution of expected compared to observed −log₁₀, P for the association test results before (red squares; λ=1.051) and after genomic control adjustment (blue diamonds; λ=1.00). The green triangles represent the distribution of −log₁₀ P values after removal of genome wide significant and correlated SNPs from the seven loci associated with PDB.

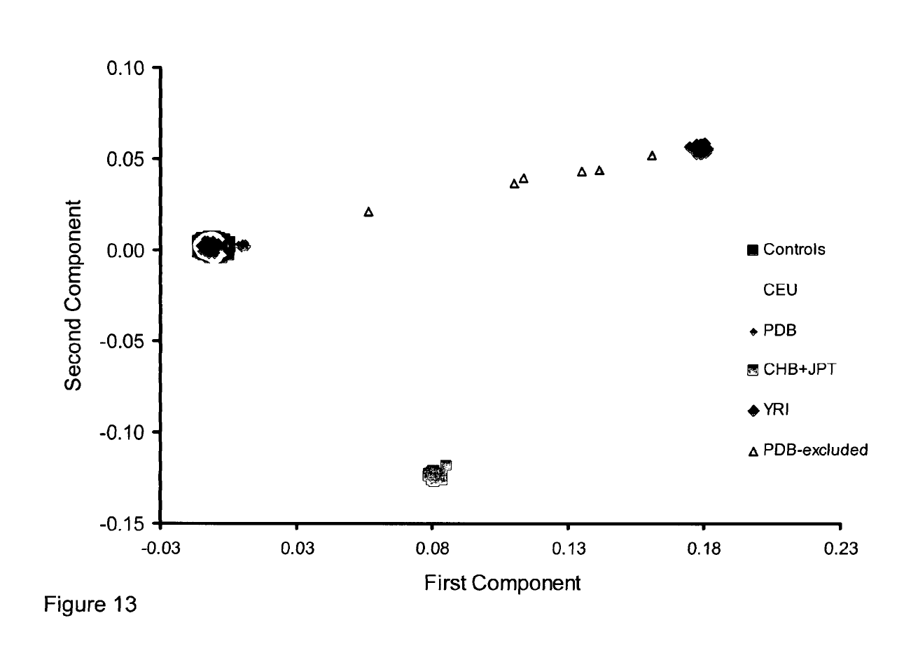

FIG. 13: Population ancestry in the GWAS sample. Multidimensional scaling analysis of IBS sharing matrix of PDB cases (purple diamonds) and controls (blue squares) including HapMap project samples of European (CEU; yellow circles), Asian (CHB+JPT; orange squares), and African (YRI; green diamonds) population. Samples located outside the main CEU cluster (grey, n=6) were excluded before testing for association with Paget's disease of bone.

### EXAMPLE 1

### Materials & Methods

### Study Subjects.

The genome-wide association study was conducted in a discovery sample of 750 cases of predominantly British descent with clinical and radiological evidence of PDB in whom mutations of the SQSTM1 gene had been excluded by DNA sequencing. These comprised subjects who had participated in the PRISM study (n=597), a randomized trial of two different treatment strategies for PDB³⁸; clinic-based subjects from the UK with sporadic PDB (n=55); and subjects with a family history of PDB derived from the UK (n=20), Australia (n=66), New Zealand (n=8) and Italy (n=4). Details of the 1,002 control subjects have previously been described⁹; in brief, they comprised healthy subjects of Scottish descent with no clinical evidence of PDB. For the replication study, we conducted genotyping in an additional 500 PDB cases without SQSTM1 mutations who were diagnosed according to standard techniques. These comprised subjects with sporadic PDB who had been recruited from hospital clinics in the UK (n=226), Italy (n=20) and Spain (n=200); subjects with sporadic PDB who had participated in the PRISM study (n=43); and subjects with a positive family history of PDB who had been recruited from hospital clinics in Australia (n=10) and the UK (n=1). The 535 replication controls comprised subjects from the UK who had been referred for investigation of osteoporosis but who had been found to have normal bone density on examination by dual energy X-ray absorptiometry (n=248), spouses of participants of the PRISM study who were not known to be affected by PDB (n=252) and clinic-based controls from Spain (n=35). All study participants were of European descent. The studies were approved by ethics review committees at the relevant institutions and all participants provided informed consent. The discovery sample had 96% power to detect disease-associated alleles with MAF=0.2 and a genotype relative risk of 1.6, assuming a multiplicative model and a disease with population prevalence of 2%.

### Stage 1 Genotyping and Quality Control.

Genotyping of PDB cases was performed at the genetics core of the Wellcome Trust Clinical Research Facility using Illumina HumanHap300-Duo BeadChip v2. Genotyping of the controls had been previously performed by Illumina Inc. using HumanHap300 v1 and HumanHap240S arrays⁹. Genotypes for cases and controls were called using BeadStudio v3.2 (Illumina, Inc.) by following the manufacturer's recommended protocol. Genotype data for control subjects were provided after applying the quality-control measures described previously⁹. For the cases, we used a no-call threshold of 0.15 in BeadStudio and quality-control metrics such as cluster separation, AB T mean (the mean normalized theta values of the heterozygote cluster) and AB R mean (the mean normalized intensity of the heterozygote cluster) to exclude badly performing SNPs. Samples with a call rate of less than 90% were excluded (n=30). The data were then subjected to further quality-control measures using PLINK³⁹ to exclude SNPs with a call rate of less than 95%, those with Hardy-Weinberg equilibrium P values of less than 1.0×10⁻⁴ in controls and those with a minor allele frequency of less than 1%. This left a total of 294,663 SNPs common to cases and controls with at least 95% call rates in each set. Samples with excess heterozygosity (1 case), non-European ancestry (21 cases and 1 control) and related subjects (6 cases) were excluded before analysis, leaving a final total of 692 cases and 1,001 controls with an average (±standard deviation) genotype call rate of 99.63±1.0. The genotype cluster plots for all SNPs showing association with PDB at P<1.0×10⁻⁴ were visually inspected in BeadStudio. Population ancestry was determined using multidimensional scaling analysis of the IBS distances matrix of all individuals after combining genotype data from the HapMap project (release 22) samples of European (CEU), Asian (CHB and JPT) and African (YRI) ancestry. For this analysis, we first removed SNPs in areas of extended LD (chr. 2: positions 134.0-138.0; chr. 6: 25.0-34.0; chr. 8: 8.0-12.0; chr. 11: 45.0-57.0)⁴⁰ and those with r²>0.2 within a 150-SNP window. SNPs with call rate <99%, MAF <5% and Hardy-Weinberg equilibrium P<1.0×10 in cases or controls were also excluded, leaving a total of 63,528 SNPs. The genome-wide average IBS distances matrix for all pairs of individuals was then calculated based on the total 63,528 SNPs using PLINK and was then used for multidimensional scaling analysis. FIG. 1 is a plot of the first two components of the multidimensional scaling analysis showing three clusters corresponding to the CEU, CHB with JPT, and YRI samples, with the majority of cases and controls located within the European CEU cluster. We identified 21 cases and 1 control as outliers from the CEU cluster, and these were excluded from further analysis. Based on genome-wide IBS distance, we identified five identical pairs (IBS distance>99%) and one related pair (IBS distance>85%) of samples from the cases cohort; the sample with the lowest call rate was excluded from each pair before further analysis.

### Stage 2 Genotyping and Quality Control.

Genotyping of replication samples was performed by Sequenom using the MassARRAY iPLEX platform. DNA from cases and controls was distributed into 384-well plates so that each plate had the same number of cases and controls to minimize genotyping bias due to variations between runs. We included 100 samples from stage 1 as a quality-control measure. The concordance rate between Illumina and Sequenom platforms was >99.9%. Replication samples with call rate <95% were excluded (19 cases and 15 controls), leaving a total of 481 cases and 520 controls with an average genotype call rate of 99.61%. The call rate of all the genotyped SNPs was >95%.

### Imputation.

Genotypes were imputed using MACH⁴¹ for untyped variants located within 2.0 Mb of SNPs identified in stage 1 as having genome-wide significant association with PDB. The HapMap CEU genotype data from release 22 were used as a reference. To avoid spurious association caused by inaccurate imputation of SNPs located at both ends of the imputed segments, analysis was restricted to SNPs located in the middle 2 Mb of each 4-Mb imputed segment. We used 200 rounds of Markov chain iterations to estimate allele dosage and the most likely genotypes of individuals in the stage 1 data. Imputation quality was assessed by estimating the correlation (r²) between imputed and true genotypes. SNPs with r<0.3 were excluded before further analysis. Analysis of imputed data was performed using logistic regression implemented in mach2dat⁴² in which the imputed allelic dosage was used to account for uncertainty in imputed genotypes.

### Statistical Analysis.

Statistical analyses were performed using PLINK (Version 1.07)³⁹. In stage 1, genotyped SNPs were tested for association with PDB using a stratified CMH test. Samples were stratified based on their genome-wide IBS similarity so that individuals assigned to one cluster were not genetically different (P>0.001, obtained from a pairwise population concordance test). The quantile-quantile plot and genomic control factor (λ) were used to assess overdispersion of the test statistics and were calculated using the statistical package R version 2.7.2 (see URLs) based on the 90% least significant SNPs as described previously10, Stepwise logistic regression was used to test for independent effects of an individual SNP, where the allelic dosage of the conditioning SNP was entered as a covariate in the regression model along with the population clusters identified by IBS-sharing analysis described above in order to adjust for population substructure. Haplotype analysis was performed by logistic regression, which looked at the presence or absence of the test haplotype and included the population clusters as a covariate in the model. Haplotypes were phased using the expectation-maximization algorithm implemented in PLINK, and only haplotypes with a frequency of ≧1% were analyzed. The cutoff point for genome-wide significance was set as P<1.7×10⁻⁷ (0.05/294,663 total SNPs) for stage 1, and P<3×10⁻³ (0.05/16 total SNPs) for the replication stage. For the combined analysis, we set the threshold for significance as P<5×10⁻⁸ as recently proposed⁴³. The replication and combined datasets were analyzed as described above except that the replication dataset was considered as a separate cluster when population clusters were used in a stratified CMH test or as a covariate in logistic regression models. The population attributable risk (PAR) for markers showing association with PDB was calculated according to the following formula:

PAR=p(OR−1)/[p(OR−1)+1]

where p is the frequency of the risk allele in controls and OR is the risk allele odds ratio. The cumulative PAR was calculated as follows:

cumulativePAR=1−(Π_{1→n}(1−PARi))

where n is the number of variants and PARi is the individual PAR for the i^{th} SNP. URLs. R, http://www.r-project.org/.

### Results & Discussion

In this study, we sought to identify genetic variants that predispose to PDB in individuals without SQSTM1 mutations by using a genome-wide association approach. In the discovery population (stage 1), we genotyped 750 cases and 1,002 controls⁹ using Illumina arrays. In the replication population (stage 2), we genotyped the most significant SNPs identified from stage 1 in an independent set of 500 cases and 535 controls using the Sequenom MassARRAY iPLEX platform. Details of the subjects used in the discovery and replication stages of the study are provided in the Online Methods.

We used a multidimensional scaling analysis of an identity-by-state (IBS) sharing matrix of all individuals plus HapMap samples to assess population ancestry (FIG. 1). After applying quality-control measures and excluding subjects with non-European ancestry, genotypic data in the discovery group were available for 294,663 SNPs in 692 cases and 1,001 controls. Association testing of the discovery stage data was performed using a stratified Cochran-Mantel-Haenszel (CMH) test in which samples were stratified according to their genome-wide IBS-sharing similarity. A quantile-quantile plot comparing the observed and expected distributions of −log 10 of P found by the CMH test showed some evidence for inflation of the test statistics given the multiple nationalities of PDB cases (genomic inflation factor¹⁰ λ=1.096; FIG. 2). To exclude the possibility of false positive association due to hidden population substructure, the observed test statistics were corrected using the genomic control method¹⁰. Six SNPs showed genome-wide significant associations with PDB after Bonferroni correction for multiple testing (FIG. 3). Three were located within a 14-kb region of chromosome 1p13.3 (rs10494112, rs499345 and rs484959), one was located on chromosome 10p13 (rs1561570) and two were located within a 22-kb region of chromosome 18q21.33 (rs2957128 and rs3018362; Table 1, FIG. 4 and Table 2). We then used genotype data from HapMap to impute SNPs located within 1.0 Mb of the six SNPs which reached genome wide significance. Association testing of imputed SNPs revealed several variants with genome-wide significance located close to the six genotyped significant SNPs identified from stage 1 (FIG. 4). The imputed SNPs showed a slightly stronger association signal than the genotyped markers due to the conservative nature of the stratified CMH test used to analyze the genotyped markers as compared to the regression methods used to test imputed markers.

In total, 76 SNPs with P values of 1×10⁻⁴ or less were identified in the discovery dataset (Table 3). From these, we selected those SNPs with P<1.0×10⁻⁶ and also those with P<1.0×10⁻⁵ in which an additional SNP within 50 kb attained a P value of 1.0×10⁻³ or less for further analysis in the replication group. Following application of quality-control measures on the replication dataset, genotype data were obtained for 481 cases and 520 controls for the 16 selected SNPs (Table 4). Eight SNPs showed significant association with PDB in the replication stage after correction for multiple testing (P<3×10⁻³), resulting in the identification of eight SNPs for which the P values attained genome-wide significance in the combined dataset (Table 4). The distribution of minor alleles and the direction of associations were similar in both the discovery and replication datasets (Table 1 and Table 4). Although all samples used in the replication stage were from individuals of European ancestry, confounding due to population substructure is possible given the multiple nationalities of the replication cohorts. To address this issue, we tested for association in replication samples from individuals of British descent only (256 cases and 488 controls) using the CMH test. This yielded results that were qualitatively similar to those obtained from the entire replication cohort (Table 5). Linkage disequilibrium (LD) patterns in the associated regions were also similar across the study samples and were similar to those observed in HapMap CEU samples (FIG. 5). Furthermore, the distribution of allele frequencies for SNPs showing genome-wide significant association to PDB was broadly similar across individuals in this study when grouped by origin (Table 6), and the replicated hits were not located in genomic regions with known geographic variation among European populations^{11,12}. This indicates that the associations reported here are unlikely to be confounded by population substructure.

Significant association with PDB was observed on chromosome 1p13.3 for three SNPs (rs10494112, rs499345 and rs484595); the strongest signal was with rs484959 (combined P=5.38×10⁻²⁴; Table 1 and Table 2). These SNPs were weakly correlated (r²<0.36) with other genotyped SNPs and are located in a 14-kb LD block 87 kb upstream of CSF1 (FIG. 4). Another gene (EPS8L3) is located 47 kb further upstream but is separated from the associated SNPs by two recombination hot spots (FIG. 4), making it a less likely candidate. Stepwise regression analysis that adjusted for population clusters and accounted for the genotypic additive effect of the three SNPs showed strong evidence for independent association with rs484959 (P=4.7×10⁻¹⁰ and rs10494112 (P=9.28×10⁻³) as compared to rs499345 (P=0.80; Table 7). This is not surprising, as rs499345 is correlated with rs10494112 (r²=0.61) but rs484959 and rs10494112 are not (r²=0.21; FIG. 5). An analysis of haplotypes formed by these three SNPs did not show a stronger association than an analysis of the individual SNPs (Table 8). CSF1 encodes macrophage colony-stimulating factor (M-CSF), which is a strong functional candidate for PDB susceptibility because it has a critical role in osteoclast formation and survival^{13,14}. Furthermore, loss-of-function mutations in rodent Csf1 cause osteopetrosis due to failure of osteoclast differentiation^{15,16}, whereas clinical studies have shown that individuals with PDB have increased serum levels of M-CSF¹⁷.

A second locus showing significant association with PDB was situated on chromosome 10p13. Three SNPs (rs1561570, rs825411 and rs2095388), all located within a 30-kb region, were analyzed in both stages of the study and the strongest signal was observed for rs1561570 (combined P=6.09×10⁻¹³; Table 1 and Table 4). These three SNPs are weakly correlated with other genotyped SNPs (highest r²<0.37). rs825411 is not in LD with rs1561570 (r²=0.04) (FIG. 5) but did attain borderline genome-wide significance (P=7.82×10⁻⁸) and appeared to have an independent association with PDB as revealed by regression analysis accounting for the genotypic additive effect of rs1561570 (P=2.23×10⁻⁹) and rs825411 (P=5.15×10⁻⁶; Table 7). Haplotype analysis showed a stronger association signal for alleles formed by both rs1561570 and rs825411 combined as compared to a single-SNP analysis, with the risk haplotype ‘TC’ showing the strongest association (P=2.50×10⁻¹⁷, OR=1.67; Table 8). The third SNP (rs2095388) showed no significant association with PDB in the combined analysis (P=3.08×10⁻⁶; Table 4) and had no independent effect after accounting for rs1561570 and rs825411 in the analysis (P=0.14; Table 7).

The 10p13 locus is marked by two recombination hot spots and contains only one known gene, OPTN (FIG. 4). It is interesting to note that this region of chromosome 10p13 has been previously linked to familial PDB but the causal gene within this region has not been identified⁶. It is therefore possible that the risk haplotype could be tagging rare allele(s) within OPTN that markedly increase susceptibility to PDB. In this regard, there have been other reports in which genome-wide association studies have identified common variants that are associated with diseases in which the associated variants lie within regions previously mapped by linkage analysis. Examples include variants associated with amyotrophic lateral sclerosis¹⁸ and Crohn's disease¹⁹. Alternatively, the risk haplotype could be tagging another common susceptibility variant within the gene. Further studies will be required to investigate these possibilities. OPTN, which encodes optineurin, is a new candidate gene for PDB. Mutations in OPTN have been linked to glaucoma²⁰, but until now, OPTN has not been implicated in regulating bone metabolism. Optineurin is a ubiquitously expressed cytoplasmic protein²¹ that contains a ubiquitin-binding domain, similar to that present in the protein NEMO. Optineurin negatively regulates TNF-α-induced NF-κB activation by interacting with ubiquitylated RIP proteins²². Furthermore, a putative NF-κB binding site has been identified in the OPTN promoter²³, and studies have shown that optineurin interacts with myosin VI, suggesting it plays a role in vesicular trafficking between the Golgi apparatus and plasma membrane²⁴. This is of interest because mutations affecting the VCP protein, which is also involved in vesicular trafficking, cause inclusion body myopathy with early-onset Paget's disease and frontotemporal dementia (IBMPFD) syndrome²⁵. Taken together, these data indicate that optineurin may have a hitherto unrecognized role in regulating bone metabolism through its effects on NF-κB signaling and/or vesicular trafficking.

The third region showing a significant association with PDB was located on chromosome 18q21.33 near TNFRSF11A, which encodes the receptor activator of NF-κB (RANK). Four SNPs within a 300-kb region reached genome-wide significance in the combined analysis (rs663354, rs2980996, rs2957128 and rs3018362). Regression analysis accounting for the genotypic-additive effect of the four SNPs showed that only rs2957128 (P=0.047) and rs3018362 (P=0.022) had independent effects (Table 7). Analysis of haplotypes formed by alleles of rs2957128 and rs3018362 showed that a risk haplotype ‘AA’ was consistently over-represented in PDB cases compared with controls in the combined sample of cases and controls (P=8.71×10⁻¹⁴, OR=1.55; Table 8). These two SNPs are moderately correlated (r²=0.55) and are located in adjacent LD blocks about 5 kb downstream of TNFRSF11A (FIG. 4c).

The TNFRSF11A gene product RANK plays a critical role in osteoclast differentiation and function. Mice with targeted disruption of Tnftsf11a exhibit severe osteopetrosis due to complete absence of osteoclasts²⁶, and loss-of-function mutations in TNFRSF11A cause osteoclast-poor osteopetrosis in humans²⁷. Mutations affecting the signal peptide region of RANK cause the PDB-like syndromes of familial expansile osteolysis, early-onset familial PDB and expansile skeletal hyperphosphatasia²⁸⁻³⁰. Mutations of TNFRSF11A have not so far been identified in individuals with classical PDB^{28,31}, although this region of chromosome 18q22 has been linked to PDB in some families³². It is also interesting to note that rs3018362 and rs884205, located downstream of TNFRSF11A, have recently been associated with bone mineral density and fracture risk³³⁻³⁵. The allele of rs3018362 that was associated with PBD was also associated with reduced bone mineral density, raising the possibility that this allele may be associated with increased bone turnover. rs884205 was not directly genotyped in our study, but it is moderately correlated with both rs3018362 (r²=0.53) and rs2957128 (r²=0.52). Imputation analysis showed evidence for association of rs884205 with PDB (imputed P value=5.93×10⁻¹¹), confirming the importance of TNFRSF11A in the genetic regulation of bone metabolism.

The three loci on chromosomes 1p13, 10p13 and 18q21 identified in this study appear to have independent roles, as we found no evidence to suggest that the associated SNPs within these loci interacted with each other to influence susceptibility to PDB (P>0.33 for all interlocus pairwise interactions; Table 9). These data are consistent with a multiplicative model for association with PDB. The cumulative population attributable risk for the SNPs showing independent association with PDB was 70%. Additionally, the risk of PDB increased with an increasing number of risk allele scores (OR_{per-risk allele}=1.34, 95% CI 1.29-1.40, P=5.81×10⁻⁴⁵), with individuals carrying ten or more risk alleles having a sixfold increase in PDB risk compared to those with the median number of risk alleles (Table 10).

It is likely that other genomic regions also contribute to PDB because the present study was powered only to detect variants with a moderate effect size (risk allele OR>1.6). A quantile-quantile plot showing the distribution of P values after removal of all genome-wide significant SNPs and correlated markers showed an excess in the number of SNPs with low P values compared to what is expected by chance (FIG. 2). For example, we observed 19 SNPs with P<1×10⁻⁵ compared to the expected 3, suggesting that other risk variants with a modest effect remain to be identified. These may include variants located on chromosomes 3p24, 8q22, 10q24 and 14q32, which did not reach genome-wide significance but could be considered suggestively associated with PDB (combined P<1×10⁻⁵; Table 4). For example, results from analysis of the 8q22 locus in an extended cohort of 1401 cases and 3199 controls confirmed the association of variants in TM7SF4 gene with PDB risk (P=9.16×10⁻¹², Table 1). TM7SF4 which encodes a dendritic cell-specific transmembrane protein (DC-STAMP) plays an essential role in osteoclast differentiation, as reflected by the fact that osteoclast fusion was completely absent in cells cultured from mice with targeted inactivation of DC-STAMP (Yagi et al 2005). Also of particular interest is the 14q32 locus containing RIN3, encoding Ras interaction/interference protein 3, which is involved in vesicular trafficking36 and could be important in osteoclast function.

In summary, we have demonstrated that common genetic variants at loci close to CSF1, OPTN, TM7SF4 and TNFRSF11A are independently associated with PDB. Further studies are now warranted to explore the mechanisms responsible for these associations.

### References for Example 1

1. Cooper, C. et al. The epidemiology of Paget's disease in Britain: is the prevalence decreasing? J. Bone Miner. Res. 14, 192-197 (1999).
2. Siris, E. S. Paget's disease of bone. J. Bone Miner. Res. 13, 1061-1065 (1998).
3. Morales-Piga, A. A., Rey-Rey, J. S., Corres-Gonzalez, J., Garcia-Sagredo, J. M. & Lopez-Abente, G. Frequency and characteristics of familial aggregation of Paget's disease of bone. J. Bone Miner. Res. 10, 663-670 (1995).
4. Laurin, N., Brown, J. P., Morissette, J. & Raymond, V. Recurrent mutation of the gene encoding sequestosome 1 (SQSTM1/p62) in Paget disease of bone. Am. J. Hum. Genet. 70, 1582-1588 (2002).
5. Hocking, L. J. et al. Domain-specific mutations in sequestosome 1 (SQSTM1) cause familial and sporadic Paget's disease. Hum. Mol. Genet. 11, 2735-2739 (2002).
6. Lucas, G. J. et al. Identification of a major locus for Paget's disease on chromosome 10p13 in families of British descent. J. Bone Miner. Res. 23, 58-63 (2008).
7. Hocking, L. J. et al. Genomewide search in familial Paget disease of bone shows evidence of genetic heterogeneity with candidate loci on chromosomes 2q36, 10p13, and 5q35. Am. J. Hum. Genet. 69, 1055-1061 (2001).
8. Laurin, N. et al. Paget disease of bone: mapping of two loci at 5q35-qter and 5q31. Am. J. Hum. Genet. 69, 528-543 (2001).
9. Tenesa, A. et al. Genome-wide association scan identifies a colorectal cancer susceptibility locus on 11q23 and replicates risk loci at 8q24 and 18q21. Nat. Genet. 40, 631-637 (2008).
10. Clayton, D. G. et al. Population structure, differential bias and genomic control in a large-scale, case-control association study. Nat. Genet. 37, 1243-1246 (2005).
11. Wellcome Trust Case Control Consortium. Genome-wide association study of 14,000 cases of seven common diseases and 3,000 shared controls. Nature 447, 661-678 (2007).
12. Novembre, J. et al. Genes mirror geography within Europe. Nature 456, 98-101 (2008).
13. Tsurukai, T., Udagawa, N., Matsuzaki, K., Takahashi, N. & Suda, T. Roles of macrophage-colony stimulating factor and osteoclast differentiation factor in osteoclastogenesis. J. Bone Miner. Metab. 18, 177-184 (2000).
14. Bouyer, P. et al. Colony-stimulating factor-1 increases osteoclast intracellular pH and promotes survival via the electroneutral Na/HCO3 cotransporter NBCn1. Endocrinology 148, 831-840 (2007).
15. Van Wesenbeeck, L. et al. The osteopetrotic mutation toothless (tl) is a loss-of-function frameshift mutation in the rat Csf1 gene: evidence of a crucial role for CSF-1 in osteoclastogenesis and endochondral ossification. Proc. Natl. Acad. Sci. USA 99, 14303-14308 (2002).
16. Wiktor-Jedrzejczak, W et al. Total absence of colony-stimulating factor 1 in the macrophage-deficient osteopetrotic (op/op) mouse. Proc. Natl. Acad. Sci. USA 87, 4828-4832 (1990).
17. Neale, S. D., Schulze, E., Smith, R. & Athanasou, N. A. The influence of serum cytokines and growth factors on osteoclast formation in Paget's disease. QJM 95, 233-240 (2002).
18. van Es, M. A. et al. Genome-wide association study identifies 19p13.3 (UNC13A) and 9p21.2 as susceptibility loci for sporadic amyotrophic lateral sclerosis. Nat. Genet. 41, 1083-1087 (2009).
19. Rioux, J. D. et al. Genome-wide association study identifies new susceptibility loci for Crohn disease and implicates autophagy in disease pathogenesis. Nat. Genet. 39, 596-604 (2007).
20. Rezaie, T. et al. Adult-onset primary open-angle glaucoma caused by mutations in optineurin. Science 295, 1077-1079 (2002).
21. Rezaie, T., Waitzman, D. M., Seeman, J. L., Kaufman, P. L. & Sarfarazi, M. Molecular cloning and expression profiling of optineurin in the rhesus monkey. Invest. Ophthalmol. Vis. Sci. 46, 2404-2410 (2005).
22. Zhu, G., Wu, C. J., Zhao, Y. & Ashwell, J. D. Optineurin negatively regulates TNFα-induced NF-κB activation by competing with NEMO for ubiquitinated RIP. Curr. Biol. 17, 1438-1443 (2007).
23. Sudhakar, C., Nagabhushana, A., Jain, N. & Swarup, G. NF-κB mediates tumor necrosis factor α-induced expression of optineurin, a negative regulator of NF-κB. PLoS One 4, e5114 (2009).
24. Sahlender, D. A. et al. Optineurin links myosin VI to the Golgi complex and is involved in Golgi organization and exocytosis. J. Cell Biol. 169, 285-295 (2005).
25. Watts, G. D. et al. Inclusion body myopathy associated with Paget disease of bone and frontotemporal dementia is caused by mutant valosin-containing protein. Nat.
Genet. 36, 377-381 (2004).
26. Li, J. et al. RANK is the intrinsic hematopoietic cell surface receptor that controls osteoclastogenesis and regulation of bone mass and calcium metabolism. Proc. Natl. Acad. Sci. USA 97, 1566-1571 (2000).
27. Villa, A., Guerrini, M. M., Cassani, B., Pangrazio, A. & Sobacchi, C. Infantile malignant, autosomal recessive osteopetrosis: the rich and the poor. Calcif. Tissue Int. 84, 1-12 (2009).
28. Hughes, A. E. et al. Mutations in TNFRSF11A, affecting the signal peptide of RANK, cause familial expansile osteolysis. Nat. Genet. 24, 45-48 (2000).
29. Nakatsuka, K., Nishizawa, Y. & Ralston, S. H. Phenotypic characterization of early onset Paget's disease of bone caused by a 27-bp duplication in the TNFRSF11A gene. J. Bone Miner. Res. 18, 1381-1385 (2003).
30. Whyte, M. P. & Hughes, A. E. Expansile skeletal hyperphosphatasia is caused by a 15-base pair tandem duplication in TNFRSF11A encoding RANK and is allelic to familial expansile osteolysis. J. Bone Miner. Res. 17, 26-29 (2002).
31. Wuyts, W. et al. Evaluation of the role of RANK and OPG genes in Paget's disease of bone. Bone 28, 104-107 (2001).
32. Hocking, L. et al. Familial Paget's disease of bone: patterns of inheritance and frequency of linkage to chromosome 18q. Bone 26, 577-580 (2000).
33. Styrkarsdottir, U. et al. Multiple genetic loci for bone mineral density and fractures. N. Engl. J. Med. 358, 2355-2365 (2008).
34. Rivadeneira, F. et al. Twenty bone-mineral-density loci identified by large-scale meta-analysis of genome-wide association studies. Nat. Genet. 41, 1199-1206 (2009).
35. Styrkarsdottir, U. et al. New sequence variants associated with bone mineral density. Nat. Genet. 41, 15-17 (2009).
36. Saito, K. et al. A novel binding protein composed of homophilic tetramer exhibits unique properties for the small GTPase Rab5. J. Biol. Chem. 277, 3412-3418 (2002).
37. Barrett, J. C., Fry, B., Maller, J. & Daly, M. J. Haploview: analysis and visualization of LD and haplotype maps. Bioinformatics 21, 263-265 (2005).
38. Langston, A. L. et al. Randomized trial of intensive bisphosphonate treatment versus symptomatic management in Paget's disease of bone. J. Bone Miner. Res. 25, 20-31 (2010).
39. Purcell, S. et al. PLINK: a tool set for whole-genome association and population-based linkage analyses. Am. J. Hum. Genet. 81, 559-575 (2007).
40. Price, A. L. et al. Long-range LD can confound genome scans in admixed populations. Am. J. Hum. Genet. 83, 132-135 (2008).
41. Li, Y. & Mach Abecasis, G. R. 1.0: Rapid haplotype reconstruction and missing genotype inference. Am. J. Hum. Genet. S79, 2290 (2006).
42. Li, Y., Willer, C., Sanna, S. & Abecasis, G. Genotype imputation. Annu. Rev. Genomics Hum. Genet. 10, 387-406 (2009).
43. Pe'er, I., Yelensky, R., Altshuler, D. & Daly, M. J. Estimation of the multiple testing burden for genomewide association studies of nearly all common variants. Genet. Epidemiol. 32, 381-385 (2008).

### Tables for Example 1 are Presented Below.

TABLE 1
SNPs showing genome-wide significant association with Paget's disease of bone

| | | | Risk | | Combined | | | | |
|---|---|---|---|---|---|---|---|---|---|
| | | | Allele | | | | Risk | | |
| | | Position | (Wild | RAF | RAF | Combined | Allele | PAR | Closest |
| Chr | SNP | (bp) | Type) | PDB | Controls | P a | OR | (%) | Gene |
| 1 | rs10494112 | 110,154,000 | G (A) | 0.298 | 0.203 | 1.67 × 10⁻¹⁴ | 1.65 | 11.7 | EPS8LS, |
| | | | | | | | | | CSF1 |
| 1 | rs484959 | 110,167,606 | G (A) | 0.651 | 0.510 | 5.38 × 10⁻²⁴ | 1.82 | 29.5 | EPS8LS, |
| | | | | | | | | | CSF1 |
| 8 | rs2458413 | 105,428,608 | A (G) | 0.659 | 0.578 | 9.16 × 10⁻¹² | 1.41 | 19.2 | TM7SF4 |
| | | | | | | | | | (DC-STAMP) |
| 10 | rs1561570 | 13,195,732 | T (C) | 0.646 | 0.545 | 6.09 × 10⁻¹³ | 1.54 | 22.7 | OPTN |
| 10 | rs825411 | 13,209,380 | C (T) | 0.598 | 0.520 | 7.82 × 10⁻⁸ | 1.35 | 15.4 | OPTN |
| 18 | rs2957128 | 58,211,715 | A (G) | 0.484 | 0.392 | 1.86 × 10⁻¹¹ | 1.46 | 15.3 | TNFRSF11A |
| 18 | rs3018362 | 58,233,073 | A (G) | 0.440 | 0.342 | 5.27 × 10⁻¹³ | 1.52 | 15.1 | TNFRSF11A |
| | | | | | | | Cumulative | 76.30% | |
| | | | | | | | PAR | | |

PAR defined as “The expected reduction in disease load after removal of a risk allele”
RAF is Risk Allele frequency, PDB is Paget's disease cases, OR is odds ratio for the risk allele PAR defined as the expected reduction in disease load after removal of a risk allele.
a Results from extended cohort of 1401 PDB cases and 3199 controls

TABLE 2
Details of SNPs showing genome-wide significant association with Paget's disease of bone.

| | | | | | Discovery | | | | | | | | | |
|---|---|---|---|---|---|---|---|---|---|---|---|---|---|---|
| | | | | | | | | | | | HWE | | Replication | |
| | | | | MAF | | MAF | | | OR | HWE | Con- | Call | | MAF |
| | | Position | Minor | HapMap | MAF | Con- | GWAS | | 95% | PDB | trols | rate | MAF | Con- |
| Chr | SNP | (bp) | Allele | CEU | PDB | trols | P a | OR | CI | P | P | (%) | PDB | trols |
| 1 | rs10494112 | 110,154,000 | G | 0.167 | 0.312 | 0.200 | 9.14 × 10⁻¹² | 1.80 | 1.53-2.12 | 0.37 | 0.20 | 99.5 | 0.277 | 0.210 |
| 1 | rs499345 | 110,163,205 | A | 0.258 | 0.396 | 0.284 | 1.13 × 10⁻¹⁰ | 1.68 | 1.44-1.95 | 0.47 | 0.70 | 98.9 | 0.400 | 0.293 |
| 1 | rs484959 | 110,167,606 | A | 0.475 | 0.355 | 0.489 | 1.15 × 10⁻¹³ | 0.56 | 0.49-0.65 | 0.8 | 0.80 | 100 | 0.340 | 0.491 |
| 10 | rs1561570 | 13,195,732 | C | 0.483 | 0.346 | 0.451 | 1.11 × 10⁻⁸ | 0.64 | 0.56-0.74 | 0.04 | 0.11 | 99.9 | 0.366 | 0.463 |
| 18 | rs2957128 | 58,211,715 | A | 0.417 | 0.499 | 0.384 | 4.21 × 10⁻¹⁰ | 1.61 | 1.40-1.86 | 0.54 | 0.25 | 98.9 | 0.463 | 0.408 |
| 18 | rs3018362 | 58,233,073 | A | 0.367 | 0.458 | 0.341 | 1.35 × 10⁻¹⁰ | 1.64 | 1.42-1.89 | 0.64 | 0.78 | 100 | 0.415 | 0.343 |

| | Replication | | | | | | | | | | | | |
|---|---|---|---|---|---|---|---|---|---|---|---|---|---|
| | | | | | HWE | | Combined | | | | | | |
| | | | OR | HWE | Con- | Call | | MAF | | | OR | | |
| | Replication | | 95% | PDB | trols | rate | MAF | Con- | Combined | | 95% | PAR | Closest |
| Chr | P | OR | CI | P | P | (%) | PDB | trols | P a | OR | CI | (%) | Gene |
| 1 | 4.34 × 10⁻⁴ | 1.45 | 1.18-1.77 | 0.25 | 0.36 | 99.9 | 0.298 | 0.203 | 1.67 × 10⁻¹⁴ | 1.65 | 1.45-1.88 | 12 | EPS8LS, |
| | | | | | | | | | | | | | CSF1 |
| 1 | 5.15 × 10⁻⁷ | 1.61 | 1.33-1.94 | 0.85 | 1.00 | 99.6 | 0.398 | 0.287 | 3.02 × 10⁻¹⁶ | 1.65 | 1.47-1.85 | 16 | EPS8LS, |
| | | | | | | | | | | | | | CSF1 |
| 1 | 7.25 × 10⁻¹² | 0.53 | 0.45-0.64 | 1.00 | 0.05 | 99.8 | 0.349 | 0.49 | 5.38 × 10⁻²⁴ | 0.55 | 0.49-0.62 | 29 | EPS8LS, |
| | | | | | | | | | | | | | CSF1 |
| 10 | 1.19 × 10⁻⁵ | 0.67 | 0.56-0.80 | 0.77 | 1.00 | 100 | 0.354 | 0.455 | 6.09 × 10⁻¹³ | 0.65 | 0.58-0.73 | 23 | OPTN |
| 18 | 1.35 × 10⁻² | 1.25 | 1.05-1.49 | 0.27 | 0.59 | 99.9 | 0.484 | 0.392 | 1.86 × 10⁻¹¹ | 1.46 | 1.30-1.63 | 15 | TNFRSF11A |
| 18 | 9.81 × 10⁻⁴ | 1.36 | 1.13-1.62 | 0.19 | 0.24 | 100 | 0.440 | 0.342 | 5.27 × 10⁻¹³ | 1.52 | 1.36-1.70 | 15 | TNFRSF11A |

MAF, minor allele frequency; PDB, Paget's disease of bone cases; OR, odds ratio for the minor allele; CI, confidence interval; PAR, population attributable risk calculated for the risk allele; HWE, Hardy-Weinberg equilibrium.
a P values from stratified Cochran-Mantel-Haenszel analysis corrected using genomic control.

TABLE 3
Results of all SNPs with P < 1.0 × 10⁻⁴ in the GWAS stage

| | | | | | | MAF | | | | |
|---|---|---|---|---|---|---|---|---|---|---|
| | | | | MAF | MAF | Con- | | | | |
| Chr | SNP | Position | Allele | Total | Cases | trol | GWAS P^{a} | OR | L95 | U95 |
| 1 | rs11578700 | 105,093,642 | C | 0.223 | 0.193 | 0.244 | 5.07 × 10⁻⁵ | 0.69 | 0.58 | 0.82 |
| 1 | rs10494112 | 110,154,000 | G | 0.246 | 0.312 | 0.200 | 9.14 × 10⁻¹² | 1.80 | 1.53 | 2.12 |
| 1 | rs499345 | 110,163,205 | A | 0.329 | 0.396 | 0.284 | 1.13 × 10⁻¹⁰ | 1.68 | 1.44 | 1.95 |
| 1 | rs484959 | 110,167,606 | A | 0.434 | 0.355 | 0.490 | 1.15 × 10⁻¹³ | 0.56 | 0.49 | 0.65 |
| 1 | rs4240529 | 111,626,267 | A | 0.279 | 0.320 | 0.251 | 8.32 × 10⁻⁶ | 1.45 | 1.24 | 1.70 |
| 1 | rs789666 | 230,753,520 | T | 0.469 | 0.430 | 0.496 | 5.11 × 10⁻⁵ | 0.74 | 0.64 | 0.85 |
| 2 | rs3771281 | 47,545,785 | T | 0.365 | 0.321 | 0.396 | 1.97 × 10⁻⁵ | 0.71 | 0.62 | 0.83 |
| 2 | rs10188090 | 47,562,657 | A | 0.368 | 0.325 | 0.398 | 5.24 × 10⁻⁵ | 0.73 | 0.63 | 0.84 |
| 2 | rs16831243 | 135,478,814 | T | 0.097 | 0.127 | 0.077 | 3.91 × 10⁻⁵ | 1.68 | 1.32 | 2.12 |
| 2 | rs7582173 | 135,479,788 | A | 0.124 | 0.157 | 0.102 | 4.10 × 10⁻⁵ | 1.59 | 1.29 | 1.97 |
| 2 | rs1869829 | 135,594,032 | C | 0.189 | 0.232 | 0.160 | 7.42 × 10⁻⁶ | 1.54 | 1.28 | 1.84 |
| 2 | rs6730157 | 135,623,558 | G | 0.255 | 0.302 | 0.222 | 1.69 × 10⁻⁵ | 1.45 | 1.24 | 1.71 |
| 2 | rs1900741 | 135,718,970 | T | 0.142 | 0.178 | 0.117 | 3.13 × 10⁻⁵ | 1.56 | 1.28 | 1.91 |
| 2 | rs1561277 | 135,808,531 | A | 0.228 | 0.276 | 0.195 | 4.95 × 10⁻⁶ | 1.51 | 1.28 | 1.79 |
| 2 | rs1446585 | 136,123,949 | C | 0.221 | 0.268 | 0.189 | 8.16 × 10⁻⁶ | 1.50 | 1.27 | 1.78 |
| 2 | rs1374330 | 136,137,378 | G | 0.101 | 0.129 | 0.081 | 4.74 × 10⁻⁵ | 1.65 | 1.31 | 2.09 |
| 2 | rs313519 | 136,155,987 | C | 0.179 | 0.220 | 0.152 | 2.64 × 10⁻⁵ | 1.51 | 1.26 | 1.82 |
| 2 | rs313528 | 136,162,339 | A | 0.181 | 0.224 | 0.151 | 5.02 × 10⁻⁶ | 1.56 | 1.30 | 1.87 |
| 2 | rs6739713 | 136,205,448 | G | 0.168 | 0.209 | 0.139 | 6.25 × 10⁻⁶ | 1.58 | 1.31 | 1.90 |
| 2 | rs1438307 | 136,215,636 | C | 0.179 | 0.220 | 0.151 | 2.33 × 10⁻⁵ | 1.52 | 1.26 | 1.83 |
| 2 | rs6430585 | 136,223,397 | A | 0.144 | 0.184 | 0.116 | 1.78 × 10⁻⁶ | 1.67 | 1.36 | 2.04 |
| 2 | rs9287442 | 136,239,180 | A | 0.170 | 0.215 | 0.141 | 2.25 × 10⁻⁶ | 1.61 | 1.33 | 1.94 |
| 2 | rs1469996 | 136,259,030 | G | 0.142 | 0.181 | 0.115 | 3.75 × 10⁻⁶ | 1.65 | 1.35 | 2.02 |
| 2 | rs2322659 | 136,272,129 | T | 0.183 | 0.225 | 0.154 | 5.31 × 10⁻⁶ | 1.56 | 1.30 | 1.87 |
| 2 | rs309160 | 136,401,698 | A | 0.203 | 0.247 | 0.173 | 1.99 × 10⁻⁵ | 1.49 | 1.25 | 1.78 |
| 2 | rs309137 | 136,482,421 | C | 0.204 | 0.245 | 0.176 | 5.45 × 10⁻⁵ | 1.46 | 1.22 | 1.74 |
| 2 | rs932206 | 136,541,742 | G | 0.348 | 0.404 | 0.310 | 1.31 × 10⁻⁶ | 1.47 | 1.26 | 1.70 |
| 2 | rs1427602 | 137,245,717 | A | 0.341 | 0.383 | 0.312 | 5.24 × 10⁻⁵ | 1.38 | 1.19 | 1.60 |
| 3 | rs13092967 | 18,100,506 | A | 0.065 | 0.088 | 0.049 | 3.49 × 10⁻⁶ | 1.95 | 1.48 | 2.58 |
| 3 | rs4688903 | 18,135,064 | T | 0.083 | 0.111 | 0.064 | 5.11 × 10⁻⁶ | 1.81 | 1.41 | 2.32 |
| 3 | rs9840549 | 102,033,340 | A | 0.371 | 0.417 | 0.339 | 1.41 × 10⁻⁵ | 1.40 | 1.21 | 1.62 |
| 4 | rs11248060 | 954,359 | T | 0.106 | 0.076 | 0.127 | 1.16 × 10⁻⁵ | 0.57 | 0.45 | 0.73 |
| 4 | rs4543123 | 38,468,919 | C | 0.154 | 0.190 | 0.129 | 2.01 × 10⁻⁵ | 1.56 | 1.28 | 1.89 |
| 4 | rs4833095 | 38,476,105 | C | 0.160 | 0.196 | 0.134 | 1.80 × 10⁻⁵ | 1.55 | 1.28 | 1.88 |
| 4 | rs10516593 | 114,298,261 | A | 0.365 | 0.325 | 0.393 | 3.55 × 10⁻⁵ | 0.72 | 0.62 | 0.84 |
| 4 | rs4279237 | 123,228,624 | G | 0.197 | 0.233 | 0.172 | 4.97 × 10⁻⁵ | 1.46 | 1.23 | 1.74 |
| 5 | rs957778 | 170,625,559 | G | 0.141 | 0.171 | 0.120 | 3.85 × 10⁻⁵ | 1.54 | 1.26 | 1.88 |
| 5 | rs2914341 | 170,635,683 | T | 0.140 | 0.170 | 0.120 | 4.12 × 10⁻⁵ | 1.54 | 1.26 | 1.88 |
| 5 | rs10516081 | 170,645,136 | T | 0.142 | 0.172 | 0.120 | 2.15 × 10⁻⁵ | 1.56 | 1.28 | 1.90 |
| 6 | rs9367903 | 16,334,247 | G | 0.338 | 0.386 | 0.306 | 9.88 × 10⁻⁶ | 1.42 | 1.22 | 1.65 |
| 6 | rs622936 | 126,188,404 | C | 0.292 | 0.255 | 0.317 | 6.63 × 10⁻⁵ | 0.71 | 0.61 | 0.84 |
| 6 | rs6570952 | 149,559,776 | G | 0.319 | 0.276 | 0.348 | 6.83 × 10⁻⁵ | 0.72 | 0.62 | 0.84 |
| 6 | rs652720 | 149,581,957 | A | 0.456 | 0.408 | 0.489 | 3.35 × 10⁻⁵ | 0.73 | 0.63 | 0.84 |
| 6 | rs6903203 | 161,808,401 | A | 0.094 | 0.121 | 0.076 | 1.74 × 10⁻⁵ | 1.71 | 1.35 | 2.16 |
| 8 | rs2458413 | 105,428,608 | G | 0.374 | 0.323 | 0.409 | 6.71 × 10⁻⁶ | 0.70 | 0.60 | 0.81 |
| 9 | rs1322155 | 10,260,636 | A | 0.312 | 0.353 | 0.285 | 5.42 × 10⁻⁵ | 1.39 | 1.19 | 1.62 |
| 9 | rs10991089 | 105,874,130 | A | 0.195 | 0.159 | 0.220 | 7.45 × 10⁻⁶ | 0.64 | 0.54 | 0.77 |
| 10 | rs1077685 | 12,532,385 | T | 0.075 | 0.099 | 0.059 | 1.96 × 10⁻⁵ | 1.80 | 1.39 | 2.34 |
| 10 | rs7921853 | 13,192,672 | G | 0.499 | 0.455 | 0.529 | 4.78 × 10⁻⁵ | 0.73 | 0.64 | 0.85 |
| 10 | rs1561570 | 13,195,732 | C | 0.408 | 0.346 | 0.451 | 1.11 × 10⁻⁸ | 0.64 | 0.56 | 0.74 |
| 10 | rs2095388 | 13,224,051 | G | 0.254 | 0.214 | 0.281 | 9.86 × 10⁻⁵ | 0.71 | 0.60 | 0.84 |
| 10 | rs477950 | 100,312,648 | T | 0.075 | 0.099 | 0.059 | 9.37 × 10⁻⁶ | 1.87 | 1.43 | 2.44 |
| 10 | rs535182 | 100,314,323 | T | 0.075 | 0.097 | 0.059 | 1.33 × 10⁻⁵ | 1.85 | 1.42 | 2.42 |
| 10 | rs551674 | 100,322,286 | G | 0.073 | 0.094 | 0.059 | 4.65 × 10⁻⁵ | 1.79 | 1.36 | 2.34 |
| 10 | rs605591 | 100,324,229 | C | 0.074 | 0.095 | 0.059 | 3.09 × 10⁻⁵ | 1.80 | 1.38 | 2.36 |
| 11 | rs10501600 | 85,287,207 | C | 0.426 | 0.379 | 0.458 | 2.26 × 10⁻⁵ | 0.72 | 0.63 | 0.83 |
| 11 | rs7105817 | 86,807,548 | A | 0.092 | 0.120 | 0.073 | 5.16 × 10⁻⁵ | 1.66 | 1.31 | 2.11 |
| 11 | rs2303663 | 129,259,110 | G | 0.412 | 0.370 | 0.441 | 3.43 × 10⁻⁵ | 0.73 | 0.63 | 0.84 |
| 12 | rs7137207 | 97,317,545 | A | 0.177 | 0.148 | 0.198 | 4.51 × 10⁻⁵ | 0.66 | 0.55 | 0.80 |
| 13 | rs6490370 | 28,684,977 | A | 0.203 | 0.234 | 0.181 | 2.34 × 10⁻⁵ | 1.48 | 1.24 | 1.75 |
| 14 | rs10498635 | 92,173,062 | T | 0.171 | 0.134 | 0.197 | 3.75 × 10⁻⁶ | 0.62 | 0.51 | 0.75 |
| 16 | rs276639 | 13,086,517 | T | 0.043 | 0.025 | 0.056 | 4.69 × 10⁻⁶ | 0.38 | 0.26 | 0.57 |
| 16 | rs17532886 | 50,026,847 | G | 0.206 | 0.244 | 0.180 | 9.02 × 10⁻⁶ | 1.51 | 1.27 | 1.79 |
| 16 | rs2550360 | 56,781,791 | C | 0.180 | 0.143 | 0.206 | 3.52 × 10⁻⁵ | 0.66 | 0.55 | 0.80 |
| 16 | rs2550363 | 56,785,484 | T | 0.260 | 0.219 | 0.288 | 5.55 × 10⁻⁵ | 0.70 | 0.60 | 0.83 |
| 17 | rs11653635 | 69,002,398 | T | 0.051 | 0.071 | 0.038 | 2.79 × 10⁻⁵ | 1.98 | 1.45 | 2.70 |
| 18 | rs972697 | 26,364,017 | C | 0.191 | 0.222 | 0.169 | 5.47 × 10⁻⁵ | 1.47 | 1.23 | 1.75 |
| 18 | rs9636100 | 57,862,290 | A | 0.430 | 0.481 | 0.396 | 6.13 × 10⁻⁶ | 1.41 | 1.23 | 1.63 |
| 18 | rs4941107 | 57,902,311 | A | 0.395 | 0.445 | 0.360 | 3.07 × 10⁻⁶ | 1.43 | 1.24 | 1.65 |
| 18 | rs663354 | 57,905,270 | T | 0.272 | 0.314 | 0.244 | 1.81 × 10⁻⁵ | 1.43 | 1.23 | 1.68 |
| 18 | rs677295 | 57,912,636 | T | 0.266 | 0.306 | 0.239 | 3.40 × 10⁻⁵ | 1.42 | 1.21 | 1.67 |
| 18 | rs2980996 | 58,130,115 | C | 0.272 | 0.315 | 0.242 | 7.45 × 10⁻⁶ | 1.46 | 1.25 | 1.71 |
| 18 | rs2957128 | 58,211,715 | A | 0.430 | 0.499 | 0.384 | 4.21 × 10⁻¹⁰ | 1.61 | 1.40 | 1.86 |
| 18 | rs3018362 | 58,233,073 | A | 0.389 | 0.458 | 0.341 | 1.35 × 10⁻¹⁰ | 1.64 | 1.42 | 1.89 |
| 22 | rs134458 | 48,302,537 | T | 0.359 | 0.312 | 0.391 | 2.26 × 10⁻⁵ | 0.71 | 0.62 | 0.83 |
| X | rs6621809 | 103,444,342 | G | 0.340 | 0.390 | 0.306 | 5.71 × 10⁻⁵ | 1.45 | 1.22 | 1.72 |

MAF, minor allele frequency; OR, odds ratio and it's 95% lower (L95) and upper (U95) confidence interval.
^{a}Genomic-control adjusted P value from stratified Cochran-Mantel-Haenszel test.

TABLE 4
Details of SNPs analysed in the discovery and replication cohorts.

| | | Position | | Discovery | | | Replication | |
|---|---|---|---|---|---|---|---|---|
| Chr | SNP | (bp) | Allele | MAF | P^{a} | OR (95% CI) | MAF | P |
| 1 | rs10494112 | 110154000 | G | 0.246 | 9.14 × 10⁻¹² | 1.80(1.53-2.12) | 0.242 | 4.34 × 10⁻⁴ |
| 1 | rs499345 | 110163205 | A | 0.329 | 1.13 × 10⁻¹⁰ | 1.68(1.44-1.95) | 0.345 | 5.15 × 10⁻⁷ |
| 1 | rs484959 | 110167606 | A | 0.434 | 1.15 × 10⁻¹³ | 0.56(0.49-0.65) | 0.418 | 7.25 × 10⁻¹² |
| 3 | rs4688903 | 18135064 | T | 0.083 | 5.11 × 10⁻⁶ | 1.81(1.41-2.31) | 0.072 | 0.239 |
| 8 | rs2458413 | 105428608 | G | 0.374 | 6.71 × 10⁻⁶ | 0.70(0.60-0.81) | 0.393 | 2.07 × 10⁻² |
| 10 | rs1561570 | 13195732 | C | 0.408 | 1.11 × 10⁻⁸ | 0.64(0.56-0.74) | 0.416 | 1.19 × 10⁻⁵ |
| 10 | rs825411 | 13209380 | T | 0.441 | 1.12 × 10⁻³ | 0.78(0.67-0.90) | 0.456 | 1.96 × 10⁻⁵ |
| 10 | rs2095388 | 13224051 | G | 0.254 | 9.86 × 10⁻⁵ | 0.71(0.60-0.84) | 0.262 | 1.02 × 10⁻² |
| 10 | rs477950 | 100312648 | T | 0.075 | 9.37 × 10⁻⁶ | 1.87(1.43-2.43) | 0.070 | 0.315 |
| 14 | rs10498635 | 92173062 | T | 0.171 | 3.75 × 10⁻⁶ | 0.62(0.51-0.75) | 0.165 | 7.94 × 10⁻³ |
| 18 | rs9636100 | 57862290 | A | 0.430 | 6.13 × 10⁻⁶ | 1.41(1.22-1.63) | 0.421 | 5.01 × 10⁻² |
| 18 | rs4941107 | 57902311 | A | 0.395 | 3.07 × 10⁻⁶ | 1.43(1.24-1.65) | 0.383 | 1.88 × 10⁻² |
| 18 | rs663354 | 57905270 | T | 0.272 | 1.81 × 10⁻⁵ | 1.43(1.22-1.68) | 0.280 | 3.47 × 10⁻⁴ |
| 18 | rs2980996 | 58130115 | C | 0.272 | 7.45 × 10⁻⁶ | 1.46(1.24-1.70) | 0.275 | 1.25 × 10⁻³ |
| 18 | rs2957128 | 58211715 | A | 0.430 | 4.21 × 10⁻¹⁰ | 1.61(1.40-1.86) | 0.434 | 1.35 × 10⁻² |
| 18 | rs3018362 | 58233073 | A | 0.389 | 1.35 × 10⁻¹⁰ | 1.64(1.42-1.89) | 0.378 | 9.81 × 10⁻⁴ |

| | Replication | Combined | | | Gene |
|---|---|---|---|---|---|
| Chr | OR (95% CI) | MAF | P^{a} | OR (95% CI) | region |
| 1 | 1.45(1.18-1.77) | 0.244 | 1.67 × 10⁻¹⁴ | 1.65(1.45-1.88) | EPS8LS, |
| | | | | | CSF1 |
| 1 | 1.61(1.33-1.94) | 0.335 | 3.02 × 10⁻¹⁶ | 1.65(1.47-1.85) | EPS8LS, |
| | | | | | CSF1 |
| 1 | 0.53(0.45-0.64) | 0.428 | 5.38 × 10⁻²⁴ | 0.55(0.49-0.62) | EPS8LS, |
| | | | | | CSF1 |
| 3 | 1.23(0.87-1.72) | 0.079 | 2.48 × 10⁻⁶ | 1.57(1.29-1.92) | SATB1 |
| 8 | 0.81(0.67-0.97) | 0.381 | 4.14 × 10⁻⁷ | 0.74(0.66-0.83) | TM7SF4 |
| 10 | 0.67(0.56-0.80) | 0.411 | 6.09 × 10⁻¹³ | 0.65(0.58-0.73) | OPTN |
| 10 | 0.68(0.57-0.81) | 0.446 | 7.82 × 10⁻⁸ | 0.74(0.66-0.83) | OPTN |
| 10 | 0.77(0.63-0.94) | 0.257 | 3.08 × 10⁻⁶ | 0.73(0.64-0.83) | OPTN |
| 10 | 1.19(0.85-1.68) | 0.073 | 5.53 × 10⁻⁶ | 1.57(1.28-1.94) | HPSE2 |
| 14 | 0.72(0.57-0.92) | 0.169 | 9.69 × 10⁻⁸ | 0.66(0.56-0.76) | RIN3 |
| 18 | 1.20(1.00-1.43) | 0.427 | 8.43 × 10⁻⁷ | 1.32(1.18-1.48) | PIGN |
| 18 | 1.25(1.04-1.51) | 0.391 | 1.75 × 10⁻⁷ | 1.36(1.22-1.53) | PIGN |
| 18 | 1.43(1.18-1.74) | 0.275 | 2.36 × 10⁻⁸ | 1.43(1.27-1.62) | PIGN |
| 18 | 1.38(1.14-1.68) | 0.273 | 3.35 × 10⁻⁸ | 1.43(1.26-1.61) | TNFRSF11A |
| 18 | 1.25(1.05-1.49) | 0.432 | 1.86 × 10⁻¹¹ | 1.46(1.30-1.63) | TNFRSF11A |
| 18 | 1.36(1.13-1.62) | 0.385 | 5.27 × 10⁻¹³ | 1.52(1.36-1.70) | TNFRSF11A |

MAF, minor allele frequency; OR, odds ratio for the minor allele; CI, confidence interval.
^{a}Genomic-control adjusted P values from association testing using stratified Cochran-Mantel-Haenszel test.

TABLE 5
Details of SNPs analysed in the replication cohort of British descent.

| | | Position | Minor Allele Frequency | | | | | | | HWE P | |
|---|---|---|---|---|---|---|---|---|---|---|---|
| CHR | SNP | (bp) | Allele | Cases | Control | Total | P^{a} | OR | OR 95% CI | Cases | Controls |
| 1 | rs10494112 | 110154000 | G | 0.297 | 0.203 | 0.235 | 4.90 × 10⁻⁵ | 1.66 | 1.30-2.12 | 0.10 | 0.33 |
| 1 | rs499345 | 110163205 | A | 0.380 | 0.284 | 0.317 | 1.41 × 10⁻⁴ | 1.55 | 1.24-1.95 | 1.00 | 0.91 |
| 1 | rs484959 | 110167606 | A | 0.385 | 0.506 | 0.464 | 8.33 × 10⁻⁶ | 0.61 | 0.49-0.76 | 0.69 | 0.09 |
| 3 | rs4688903 | 18135064 | T | 0.082 | 0.069 | 0.073 | 0.347 | 1.21 | 0.81-1.81 | 0.39 | 1.00 |
| 8 | rs2458413 | 105428608 | G | 0.360 | 0.420 | 0.399 | 0.025 | 0.78 | 0.62-0.97 | 0.89 | 0.23 |
| 10 | rs1561570 | 13195732 | C | 0.340 | 0.464 | 0.421 | 4.00 × 10⁻⁶ | 0.59 | 0.48-0.74 | 0.58 | 0.78 |
| 10 | rs825411 | 13209380 | T | 0.405 | 0.502 | 0.469 | 3.81 × 10⁻⁴ | 0.67 | 0.54-0.84 | 0.24 | 0.15 |
| 10 | rs2095388 | 13224051 | G | 0.207 | 0.284 | 0.257 | 1.27 × 10⁻³ | 0.66 | 0.51-0.85 | 0.57 | 0.74 |
| 10 | rs477950 | 100312648 | T | 0.082 | 0.065 | 0.071 | 0.241 | 1.27 | 0.85-1.91 | 0.68 | 0.14 |
| 14 | rs10498635 | 92173062 | T | 0.148 | 0.186 | 0.173 | 0.066 | 0.76 | 0.57-1.02 | 0.80 | 0.76 |
| 18 | rs9636100 | 57862290 | A | 0.460 | 0.397 | 0.419 | 0.021 | 1.29 | 1.04-1.61 | 0.61 | 0.30 |
| 18 | rs4941107 | 57902311 | A | 0.389 | 0.353 | 0.365 | 0.207 | 1.16 | 0.92-1.47 | 0.32 | 0.61 |
| 18 | rs663354 | 57905270 | T | 0.303 | 0.241 | 0.262 | 9.86 × 10⁻³ | 1.37 | 1.08-1.74 | 1.00 | 0.54 |
| 18 | rs2980996 | 58130115 | C | 0.293 | 0.239 | 0.257 | 0.023 | 1.32 | 1.04-1.68 | 0.88 | 0.45 |
| 18 | rs2957128 | 58211715 | A | 0.492 | 0.414 | 0.441 | 3.88 × 10⁻³ | 1.37 | 1.11-1.70 | 0.62 | 0.58 |
| 18 | rs3018362 | 58233073 | A | 0.440 | 0.344 | 0.377 | 3.20 × 10⁻⁴ | 1.49 | 1.20-1.86 | 0.25 | 0.37 |

^{a}Replication samples of British descent (256 cases and 488 controls) were tested for association with Paget's disease using Cochran-Mantel-Haenszel test.
Odds ratio OR) and 95% confidence interval (CI) are shown for each SNP tested in the replication stage. HWE, Hardy-Weinberg equilibrium.

TABLE 6
Distribution of SNP allele frequency in subjects grouped by origin.

| | | | MAF in Cases | | | |
|---|---|---|---|---|---|---|
| | | | Australia | | | MAF in |
| | | | & New | Italy & | | Controls |
| | | Minor | Zealand | Spain | UK | UK |
| Chr | SNP^{a} | Allele | n = 72 | n = 220 | n = 881 | n = 1489 |
| 1 | rs10494112 | G | 0.317 | 0.260 | 0.301 | 0.201 |
| 1 | rs499345 | A | 0.386 | 0.432 | 0.390 | 0.284 |
| 1 | rs484959 | A | 0.340 | 0.286 | 0.364 | 0.495 |
| 10 | rs1561570 | C | 0.408 | 0.402 | 0.337 | 0.455 |
| 18 | rs663354 | T | 0.333 | 0.327 | 0.310 | 0.243 |
| 18 | rs2980996 | C | 0.329 | 0.320 | 0.309 | 0.241 |
| 18 | rs2957128 | A | 0,450 | 0.427 | 0.502 | 0.394 |
| 18 | rs3018362 | A | 0.410 | 0.382 | 0.458 | 0.342 |

MAF, minor allele frequency.
^{a}Results are based on the combined dataset for the SNPs showing genome wide significant association with PDB.

TABLE 7
Regression analysis to test for independent association

| Test SNP | Conditioned on | Test SNP P | Conditioned SNP P |
|---|---|---|---|
| Chr1: rs484959 | Chr1: rs10494112 | 4.7 × 10⁻¹⁰ | 9.28 × 10⁻³ |
| | Chr1: rs499345 | | 0.799 |
| Chr10: rs1561570 | Chr10: rs825411 | 2.23 × 10⁻⁹ | 5.15 × 10⁻⁶ |
| | Chr10: rs2095388 | | 0.144 |
| Chr18: rs3018362 | Chr18: rs9636100 | 0.022 | 0.706 |
| | Chr18: rs4941107 | | 0.553 |
| | Chr18: rs663354 | | 0.995 |
| | Chr18: rs2980996 | | 0.413 |
| | Chr18: rs2957128 | | 0.047 |

SNPs reaching genome wide significance from each locus on chromosome 1p13, 10p13 and 18q21 were tested for their independent effect using logistic regression analysis. Results shown here are from the combined dataset. The allelic dosage of conditioned SNPs was entered as a covariate in the regression model along with population clusters.

TABLE 8
Haplotype analysis of regions associated with Paget's disease of bone on chr1, 10 and 18.

| | | GWAS | | | Replication | | | | Combined | | | |
|---|---|---|---|---|---|---|---|---|---|---|---|---|
| | HF | HF | | | HF | HF | | | HF | HF | | |
| Haplotype | Cases | Controls | OR | P value | Cases | Controls | OR | P value | Cases | Controls | OR | P value |

Chr 1: rs10494112, rs499345, rs484959

| | | | | | | | | | | | | |
|---|---|---|---|---|---|---|---|---|---|---|---|---|
| ACA | 0.350 | 0.486 | 0.57 | 3.35 × 10⁻¹⁴ | 0.336 | 0.487 | 0.54 | 4.34 × 10⁻¹¹ | 0.344 | 0.486 | 0.56 | 1.09 × 10⁻²³ |
| GAG | 0.310 | 0.194 | 1.88 | 3.69 × 10⁻¹⁴ | 0.274 | 0.204 | 1.46 | 3.38 × 10⁻⁴ | 0.295 | 0.197 | 1.71 | 2.73 × 10⁻¹⁶ |
| AAG | 0.085 | 0.091 | 0.93 | 0.570 | 0.127 | 0.091 | 1.42 | 0.014 | 0.103 | 0.091 | 1.13 | 0.197 |
| ACG | 0.255 | 0.229 | 1.15 | 0.094 | 0.264 | 0.218 | 1.29 | 0.017 | 0.258 | 0.225 | 1.19 | 0.007 |

Chr 10: rs1561570, rs825411

| | | | | | | | | | | | | |
|---|---|---|---|---|---|---|---|---|---|---|---|---|
| CT | 0.188 | 0.260 | 0.60 | 1.26 × 10⁻⁷ | 0.199 | 0.267 | 0.62 | 9.12 × 10⁻⁵ | 0.193 | 0.262 | 0.61 | 6.29 × 10⁻¹¹ |
| TT | 0.212 | 0.209 | 1.01 | 0.880 | 0.208 | 0.235 | 0.84 | 0.126 | 0.210 | 0.218 | 0.94 | 0.359 |
| CC | 0.158 | 0.191 | 0.75 | 0.006 | 0.169 | 0.195 | 0.81 | 0.096 | 0.162 | 0.193 | 0.77 | 0.001 |
| TC | 0.443 | 0.340 | 1.62 | 2.74 × 10⁻¹⁰ | 0.425 | 0.303 | 1.73 | 1.73 × 10⁻⁸ | 0.436 | 0.327 | 1.67 | 2.50 × 10⁻¹⁷ |

Chr 18: rs2957128, rs3018362

| | | | | | | | | | | | | |
|---|---|---|---|---|---|---|---|---|---|---|---|---|
| AA | 0.417 | 0.298 | 1.68 | 4.46 × 10⁻¹² | 0.379 | 0.310 | 1.36 | 0.001 | 0.401 | 0.302 | 1.55 | 8.71 × 10⁻¹⁴ |
| GA | 0.034 | 0.043 | 0.78 | 0.190 | 0.035 | 0.033 | 1.08 | 0.767 | 0.034 | 0.039 | 0.87 | 0.367 |
| AG | 0.083 | 0.086 | 0.96 | 0.740 | 0.083 | 0.097 | 0.84 | 0.265 | 0.083 | 0.090 | 0.91 | 0.366 |
| GG | 0.467 | 0.573 | 0.65 | 1.72 × 10⁻⁹ | 0.502 | 0.559 | 0.80 | 0.011 | 0.482 | 0.569 | 0.70 | 2.46 × 10⁻¹⁰ |

HF; Haplotype frequency, OR; odds ratio for the presence versus absence of each haplotype.
^{a}Odds ratio and P value were calculated using logistic regression adjusting for population clusters.

TABLE 9
Pair wise SNP interaction results.

| Locus 1 | SNP1 | Locus 2 | SNP2 | OR | P^{a} |
|---|---|---|---|---|---|
| Chr 1 | rs10494112 | Chr 10 | rs1561570 | 1.03 | 0.789 |
| Chr 1 | rs10494112 | Chr 10 | rs825411 | 0.99 | 0.947 |
| Chr 1 | rs10494112 | Chr 18 | rs2957128 | 0.98 | 0.851 |
| Chr 1 | rs10494112 | Chr 18 | rs3018362 | 1.01 | 0.900 |
| Chr 1 | rs484959 | Chr 10 | rs1561570 | 0.95 | 0.550 |
| Chr 1 | rs484959 | Chr 10 | rs825411 | 1.02 | 0.775 |
| Chr 1 | rs484959 | Chr 18 | rs2957128 | 1.08 | 0.332 |
| Chr 1 | rs484959 | Chr 18 | rs3018362 | 1.08 | 0.341 |
| Chr 10 | rs1561570 | Chr 18 | rs2957128 | 0.94 | 0.441 |
| Chr 10 | rs1561570 | Chr 18 | rs3018362 | 0.95 | 0.516 |
| Chr 10 | rs825411 | Chr 18 | rs2957128 | 1.08 | 0.338 |
| Chr 10 | rs825411 | Chr 18 | rs3018362 | 0.99 | 0.916 |

^{a}Pair-wise SNP interaction for SNPs showing independent association from each locus. Analysis was performed using the combined dataset. OR is odds ratio for interaction.

TABLE 10
Cumulative contribution of identified variants to PDB risk

| | Allele score | | | |
|---|---|---|---|---|
| | frequency (%) | | | |
| Risk allele score^{a} | Cases | Controls | OR^{b} (95% CI) | P |
| 0, 1 | 0.94 | 3.02 | 0.41 (0.21-0.82) | 9.00 × 10⁻³ |
| 2 | 1.71 | 6.57 | 0.34 (0.21-0.58) | 2.81 × 10⁻⁵ |
| 3 | 6.99 | 12.49 | 0.74 (0.54-1.02) | 0.07 |
| 4 | 11.59 | 18.67 | 0.83 (0.63-1.09) | 0.17 |
| 5 | 15.17 | 20.18 | 1.00 (0.77-1.30) | 1.00 |
| 6 | 18.33 | 16.90 | 1.44 (1.11-1.87) | 5.00 × 10⁻³ |
| 7 | 19.10 | 11.18 | 2.27 (1.73-2.98) | 2.25 × 10⁻⁹ |
| 8 | 13.73 | 6.51 | 2.81 (2.05-3.83) | 4.42 × 10⁻¹¹ |
| 9 | 6.39 | 3.16 | 2.69 (1.79-4.05) | 1.07 × 10⁻⁶ |
| 10 | 4.43 | 0.99 | 5.98 (3.27-10.93) | 1.60 × 10⁻¹⁰ |
| 11, 12 | 1.62 | 0.33 | 6.55 (2.40-17.85) | 3.06 × 10⁻⁵ |

^{a}Risk allele score of the six SNPs showing independent association with PDB in the combined dataset (chr.1: rs10494112, rs484959; chr. 10: rs1561570 and rs825411; chr. 18: rs2957128 and rs3018362). Allele scores were normally distributed in cases and controls. Individuals carrying low-frequency scores (allele scores labeled 0 and 1 and 11 and 12) were combined together.
^{b}ORs are relative to the median number of risk alleles in the controls (five risk alleles).

### EXAMPLE 2

### Materials & Methods

### GWAS Stage Study Subjects.

This study describes an extension to our previously reported GWAS of PDB in which we used genotype data from 692 PDB cases from our previously described study¹, and extended the case group by genotyping an additional 57 PDB cases. The additional cases were selected from recently recruited subjects in the PRISM study²³; a randomised trial of two different treatment strategies for PDB patients from the UK. We also increased the size of the control group by using genotype data from 2,930 subjects from the British 1958 Birth Cohort genotyped by the Wellcome Trust Case-Control Consortium⁷. This control group represents a better match to our PDB cases than the previous controls which were recruited from Scotland¹ since, like the PRISM participants, they were recruited from all over the UK. The extended samples size used in this study provided 90% power to detect disease associated allele with MAF=0.2 and genotype relative risk of 1.4 assuming a multiplicative model and a disease with population prevalence of 2%. This represents a substantial increase in power compared to our previous study¹ where we had 20% power to detect alleles with genotyped relative risk of 1.4.

GWAS stage genotyping and quality control. Genotyping and quality control for the 692 PDB cases were performed using Illumina HumanHap300-Duo arrays as described previously¹. The additional 57 PDB cases were genotyped using Illumina Human660W Quad version 1 arrays and quality control measures were applied as previously described¹. Briefly; SNPs with call rate <95% were excluded and samples with call rate <90% (n=1); excess heterozygosity (n=1); and non-European ancestry (n=6; Supplementary FIG. 4) were removed before analysis. The genotyping of the British 1958 Birth Cohort was previously performed by the Wellcome Trust Case-Control Consortium using the Illumina Human 1.2M Duo custom array (www.wtccc.org.uk.)⁷. For the control group, SNPs with call rate <95% were excluded and we removed 231 samples because they failed at least one of the following quality control criteria: low call rate, non-European ancestry, gender mismatch, or cryptic relatedness. Population ancestry was determined using multidimensional scaling analysis of identity-by-state (IBS) distances matrix as previously described¹. After quality control, we analysed 741 PDB cases and 2,699 controls with genotype data for 290,115 SNPs which were common to the three different genotyping arrays. To ensure consistent genotyping between different platforms, a subset of samples were genotyped using at least two different platforms and cross-platform genotype concordance rate was >99.7% (Table 5 (Example 2)). Additionally, the genotype cluster plots for all SNPs showing association with PDB at P<1.0×10⁻⁴ were visually inspected in cases and controls and only high quality genotype data were included in the analysis. Furthermore, genotype call rate for the top associated SNPs was consistent between cases and controls (Table 6 (Example 2)).

### Replication Samples.

The replication study groups were derived from clinic-based PDB patients and gender-matched controls selected from the same region. Patients with SQSTM1 mutations were excluded and all study participants provided informed consent. The first replication cohort comprised 175 PDB patients from the UK; 8 PDB cases from Sydney Australia and 215 PDB cases from Western Australia. These patients were of British descent and were matched with 485 unaffected British controls. The second replication cohort (Italian replication cohort 1) comprised 354 PDB cases and 390 unaffected controls enrolled from various referral centres in Italy who took part in the GenPage project²⁴. The third replication cohort (Italian replication cohort 2) comprised 205 Italian PDB cases and 238 unaffected controls enrolled from referral centres in Northern, Central and Southern Italy as previously described²⁵. The fourth replication cohort comprised 246 sporadic PDB patients recruited from various referrals centres in Belgium and these were matched with 263 controls with no clinical evidence of PDB as previously described⁸. The fifth replication cohort comprised 85 PDB patients and 93 controls recruited from various centres in the Netherlands as described^{8,26}. The sixth replication comprised 186 sporadic PDB cases recruited from the Salamanca region in the Castilla-Leon region of Spain and 202 unaffected controls from the same region.

### Replication Sample Genotyping and Quality Control.

Genotyping of replication samples was performed by Sequenom (Hamburg, Germany) using the MassARRAY iPLEX platform. To minimize genotyping bias due to variations between runs; DNA from cases and controls from the six different replication cohorts were distributed into 384 well plates so that each plate had the same number of cases and controls. We included 4000 known genotypes as a quality control measure and the concordance rate between the genotype calls was >99.8%. We removed 64 samples due to low call rate (<90%) and the call rate for all genotyped SNPs was >95%.

### Imputation.

Genome-wide genotype imputation for autosomal SNPs was performed using MACH²⁷ and the HapMap European (CEU) phased haplotype data from release 22 were used as a reference. We excluded SNPs with poor imputation quality based on the estimated correlation between imputed and true genotypes (r²<0.3). Additionally, a subset (2%) of known genotypes were masked during imputation and then imputed genotypes were compared with true genotypes and the average per allele imputation error rate was 2.9%. Imputed SNPs were tested for association using PorbABEL software²⁸ implementing a logistic regression model in which the allelic dosage of imputed SNP was used to adjust for uncertainty in imputed genotypes.

### Statistical Analysis.

Statistical analyses were performed using PLINK (Version 1.07)²⁹ and R (v2.11.1). In GWAS stage, genotyped SNPs were tested for association with PDB using standard allelic (1.d.f) χ² statistic. We also performed association testing using regression models in which we adjusted for gender, population clusters (as determined by multidimensional scaling analysis) but results were essentially identical to those obtained from the standard allelic test reported here (data not shown). The genomic inflation factor λ was calculated based on the 90% least significant SNPs as described previously³⁰. The observed test statistic values were corrected using the genomic control method (λ=1.05; Supplementary FIG. 3). Logistic regression was used to test for independent effects of SNPs where the allelic dosage of the conditioning SNP was entered as a covariate in the regression model. The cut off point for genome wide significance was set as P<5×10⁻⁸ as recently proposed³¹. Association testing of replication data was performed in each replication cohort using standard (1.d.f) χ² statistic. To assess combined genetic effects, we performed meta-analysis of all studies using the inverse-variance method assuming fixed-effect model. We also tested random-effects model using DerSimonian-Laird method³² and between-study heterogeneity was assessed using the Cochran's Q and I² metrics. Heterogeneity was considered significant if Pₕₑₜ<0.05. The population attributable risk (PAR) for markers showing association with PDB was calculated according to the following formula:

PAR=p(OR−1)/[p(OR−1)+1];

where p is the frequency of the risk allele in controls and OR is the risk allele odds ratio. The cumulative PAR was calculated as follows:

Cumulative PAR=1−(Π_{1→n}(1−PARᵢ));

where n is the number of variants and PARᵢ is the individual PAR for the ith SNP. The proportion of familial risk attributable to the identified loci was calculated as previously described³³ assuming a multiplicative model of association and a sibling relative risk λₛ=7.0 as estimated from previous epidemiological studies²⁰. Regional association plots were generated using the locuszoom tool³⁴.
eQTL Analysis.

SNPs showing genome wide significant association with PDB (or those in strong LD; D′≧0.8) were tested for association with cis-allelic expression of gene transcripts located in the associated regions using publicly available eQTL data^{22,35-38}. Only cis-acting allelic associations located within 250 kb of either 5′ or 3′ end of the associated gene with expression P-value <1×10⁻⁵ were considered. To avoid false detection, we excluded expression data if the gene probe contained a polymorphic SNP or was located in a highly repetitive sequence.

### Results & Discussion

In Example 1 we describe the identification of susceptibility alleles for PDB at the CSF1, OPTN, and TNFRSF11A loci by a genome wide association study involving 692 PDB cases and 1,001 controls with replication cohort of 481 cases and 520 controls¹. In order to identify additional susceptibility loci for the disease, we performed an extended GWAS involving a total of 749 PDB cases of British descent in whom SQSTM1 mutations had been excluded and 2,930 British controls derived from the 1958 Birth Cohort⁷ with replication in a further 1,474 cases and 1,671 controls from six independent populations.

After applying quality control measures and excluding samples of non-European ancestry, the extended cohort (henceforth referred to as the GWAS stage) comprised 741 cases and 2,699 controls with genotype information for 290,115 SNPs, providing a 4-fold increase in power to detect loci of moderate effect size (odds ratio≧1.4) compared with our previous study¹. We then genotyped the highest ranking SNPs identified from the GWAS stage in six independent replication cohorts of SQSTM1-negative PDB cases and matched controls from the UK, Australia, Italy, Spain, the Netherlands, and Belgium. Details of the study groups are provided in the online methods section and Table 2 (Example 2). To increase SNP coverage, we performed genome wide SNP imputation for the GWAS stage samples using phased haplotype data from the HapMap project as a reference. The results of association testing of genotyped and imputed SNPs (total 2,487,078 SNPs) from the GWAS stage are shown in FIG. 6. A locus on chromosomes 8q22.3 showed genome-wide evidence of association with PDB (P<5.0×10⁻⁸) in addition to the previously identified genome wide significant loci on 1p13.3, 10p13 and 18q21.33¹. The 8q22.3 locus was suggestively associated with PDB risk in our previous study (rs2458413; P=4.14×10⁻⁷)¹ but the same SNP reached genome wide significance in the present study (rs2458413; P=7.85×10⁻¹¹; OR=1.51).

In the second stage of this study we analysed the highest ranking SNPs observed in the GWAS stage (P values of 5×10⁻⁵ or less) for replication after excluding those in linkage disequilibrium (LD; r²>0.8 or D′>0.95) with the highest ranking SNP from each region. A total of 27 SNPs were genotyped in the replication cohorts which consisted of 1,474 PDB cases from six different geographic regions and 1,671 unaffected controls from the same regions that were matched with the cases by gender as described in the online methods section and Table 2 (Example 2). A meta-analysis of data from the GWAS stage and individual replication cohorts was performed under fixed and random effects models and the results are summarised in Table 3 (Example 2). This strengthened the association with PDB for the CSF1, OPTN, and TNFRSF11A loci which were identified in our previous study¹ and confirmed the association with 8q22.3 locus which was suggestively associated with PDB in our previous GWAS and was confirmed to be associated with PDB in a small study of Belgian and Dutch subjects⁸. Furthermore, three additional genome wide significant loci on 7q33, 14q32.12, and 15q24.1 were identified in the combined data set (P<5×10⁻⁸; Table 1 (Example 2)). To assess if the reported associations were confounded by age, age of onset or recruitment centre, we performed a regression analysis using case-only data from the GWAS stage to test if any of these factors were associated with the top hits using linear regression models. The results of this analysis showed no evidence to suggest that the reported association is confounded by age (P>0.11), age of onset (P>0.10) or recruitment centre (P>0.44).

The strongest signal on 8q22.3 was with rs2458413 (combined P-value=7.38×10⁻¹⁷; OR=1.4). There was no significant heterogeneity between the study groups (I²=44.3%; Pₕₑₜ=0.10; Table 1 (Example 2), FIG. 8 and Table 3 (Example 2)) and the direction of association was similar in all cohorts. The associated region spans ˜220 kb and contains three known genes (RIMS2, TM7SF4, and DPYS) but SNPs with the highest association signal appear to cluster within an 18-kb LD block spanning the entire Transmembrane 7 superfamily member 4 gene (TM7SF4; FIG. 7 and Supplementary FIG. 1). This gene encodes dendritic cell-specific transmembrane protein (DC-STAMP)⁹ which is a strong functional candidate gene for PDB since it is required for the fusion of osteoclast precursors to form mature osteoclasts¹⁰. Previous studies have shown that RANKL induced DC-STAMP expression is essential for osteoclast formation¹¹ and in a recent study; Nishida et al showed that the connective tissue growth factor CCN2 stimulates osteoclast fusion through interaction with DC-STAMP¹². Since osteoclasts from patients with PDB are larger in size and contain more nuclei than normal osteoclasts, it seems likely that the genetic variants that predispose to PDB do so by enhancing TM7SF4 expression or by causing gain-of-function at the protein level but further studies will be required to investigate these possibilities.

The first new locus for PDB susceptibility was on 7q33 tagged by rs4294134 (combined P-value=8.45×10⁻¹⁰; OR=1.45). The direction of association was similar in all study cohorts and analysis of the combined data set showed no evidence for heterogeneity between study groups (I²=0%; Pₕₑₜ=0.83; Table 1 (Example 2), FIG. 8 and Table 4 (Example 2)). The associated region spans ˜350 kb and contains three known genes (CNOT4, NUP205, and SLC13A4) and two predicted protein coding transcripts (PL-5283 and FAM180A; FIG. 7). The strongest signal was with rs4294134, located within the 22^{nd} intron of NUP205. This gene encodes a protein called nucleoporin 205 kDa which is one of the main components of the nuclear pore complex involved in the regulation of transport between the cytoplasm and nucleus¹³. All SNPs with P<1×10⁻⁵ in the 350 kb associated region were in moderate to strong LD with rs4294134 (r² 0.5; D′ 0.95) with the exception of two SNPs (rs3110788 and rs3110794) which were poorly correlated with rs4294134 (r²≦0.21; D′≧0.95; FIG. 7). Conditional analysis in the GWAS stage indicated that the association signal appeared to be driven by rs4294134 (P=8.8×10⁻³) after adjusting for rs3110788 (P=0.31) and rs3110794 (P=0.10). None of the three genes located in this region are known to affect bone metabolism and further studies will be required to identify the functional variant(s) responsible for association with PDB.

The second new susceptibility locus was located on 14q32.12 and was tagged by rs10498635. This SNP showed borderline evidence of association with PDB in our previous study (P=9.69×10⁻⁸)¹ but reached genome-wide significance in the present study (combined P-value=2.55×10⁻¹¹; OR=1.44). Association testing showed no evidence for heterogeneity between the study groups V′=0.0%; Pₕₑₜ=0.62; Table 1 (Example 2), FIG. 8 and Table 4 (Example 2)). The 62 kb-associated region is bounded by two recombination hotspots and contains the gene RIN3 (FIG. 7) that encodes the Ras and Rab interactor 3, a protein that plays a role in vesicular trafficking through interaction with small GTPases such as Ras and Rab^{14,15}. The function of RIN3 in bone metabolism is currently unknown, but it could play a role in bone resorption in view of the importance that small GTPases play in vesicular trafficking and in osteoclast function^{16,17}. It is of interest to note that mutations affecting the VCP, a protein also involved in vesicular trafficking, cause the syndrome of inclusion body myopathy with early-onset Paget's disease and frontotemporal dementia (IBMPFD)¹⁸.

The third new susceptibility locus was located on 15q24.1 and the strongest association was with rs5742915 (combined P-value=1.60×10⁻¹⁴; OR=1.34; I²=0.0%; Pₕₑₜ=0.56; Table 1 (Example 2), FIG. 8 and Table 4 (Example 2)). The associated region is bounded by two recombination hot spots and spans ˜200 kb but a gap spanning ˜40 kb was observed in this region with no SNP coverage in the illumina arrays or the HapMap CEU population. The associated SNPs were clustered within the promyelocytic leukaemia gene (PML; FIG. 7) and the strongest signal was observed for rs5742915, which results in a phenylalanine to leucine amino acid change at codon 645 (F645L) of the PML protein. The function of PML in bone metabolism is unclear but it is known to be involved in TGF-β signalling¹⁹. Accordingly Lin et al showed that cells from pml knock out mice were resistant to TGF-β-dependent growth arrest and apoptosis, had impaired induction of TGF-β target genes, and exhibited abnormal nuclear translocation of the TGF-β signalling proteins Smad2 and Smad3¹⁹. Since TGF-β is known to play a role in the regulation of bone remodelling, it is possible that the association between PDB and PML could be mediated by an effect on TGF-β signalling, but further research will be required to investigate this possibility. The GOLGA6A gene is also located in the associated region and encodes a protein that belongs to golgin, a family of coiled-coil proteins associated with the Golgi apparatus and play a role in membrane fusion and as structural supports for the Golgi cisternae. This gene is located in the 40 kb gap region that contains a large low-copy repeat sequence, but its function in bone metabolism is unknown.

We were also able to replicate our previously reported association between variants at the CSF1, OPTN, and TNFRSF11A loci and PDB in the present study. The results of meta-analysis of the combined data set for these loci are shown in Table 1 (Example 2) and Supplementary FIG. 2 which provide conclusive evidence for association of variants at CSF1 (rs10494112; P-value=7.06×10⁻³⁵; risk-allele OR=1.72; I²=0.0%; Pₕₑₜ=0.97), OPTN (rs1561570; P-value=4.37×10⁻³⁸; risk-allele OR=1.67; I²=65.7%; Pₕₑₜ=0.01), and TNFRSF11A (rs3018362; P-value=7.98×10⁻²¹; risk-allele OR=1.45; I²=0.0%; Pₕₑₜ=0.46) with PDB. Evidence of heterogeneity between study groups was observed for rs1561570 at OPTN but this was due to differences in effect size rather than the direction of effect and the association remained genome wide significant after accounting for heterogeneity using the random effects model (P=4.34×10⁻¹²; OR=1.68). The heterogeneity was caused by larger effect size observed in the Dutch cohort (Supplementary FIG. 2) possibly due to the small sample size of this cohort. These observations provide highly robust evidence for association between these loci and PDB and extend those recently reported⁸ in the Dutch and Belgian populations which were also included in the present study.

We next wanted to determine if the identified loci on 15q24.1, 7q33 and 14q32.12 interacted with each other or with the previously identified loci on 1p13.3, 8q22.3, 10p13 and 18q21.33 to affect the risk of PDB. Pair-wise interaction analysis showed weak evidence for interaction between 7q33 (rs4294134) with 8q22.3 (rs2458413; P=0.03) and 10p13 (rs1561570; P=0.02). However, these interactions were not significant after adjusting for multiple testing and none of the other loci showed evidence for interaction (P>0.05) suggesting a multiplicative model of association with PDB risk. In order to estimate the effect size of the identified loci on the development of PDB, we calculated the proportion of familial risk explained by the genome wide significant loci in the replication sample assuming a sibling relative risk for PDB of 7.0²⁰. This showed that the proportion of familial risk explained was ˜13% which is much greater than observed for other bone diseases like osteoporosis²¹. We also estimated the cumulative population attributable risk of these loci in the replication cohort and found it to be 86% and we found that the risk of PDB increased with increasing number of risk allele scores defined by the seven loci (ORₚₑᵣ₋ᵣᵢₛₖₐₗₗₑₗₑ=1.44, 95% CI=1.38-1.51, P=5.4×10⁻⁵⁷). When allele scores were weighted according to their estimated effect size we found that subjects in the top 10% of the allele score distribution (D10; n=315) had 10.1 fold (95% CI; 7.0-14.6) increase in risk of developing PDB compared to those in the bottom 10% of the distribution (D1; n=315) from the replication dataset (FIG. 9). Although these data suggest that a large part of the genetic risk of PDB in patients without SQSTM1 mutations is accounted for by these loci, we acknowledge that the functional variants need to be identified before we can precisely estimate the contribution that these loci make to the risk of developing PDB. To assess the functional effect of the identified SNPs on gene expression, we tested the association between top PDB-associated SNPs (or those in LD; D′≧0.8) from each of the seven loci and cis-allelic expression of genes located in the associated regions using publicly available expression quantitative trait loci (eQTL) data. This showed highly significant associations for transcripts of TM7SF4 (rs2458415; expression P-value=1.22×10⁻¹⁸) and OPTN (rs1561570; expression P-value=6.61×10⁻⁶²) in peripheral blood monocytes²² suggesting that the association with PDB risk for these loci could be mediated by influencing gene expression levels.

In addition to the loci mentioned above, additional variants were identified that showed suggestive evidence for association with PDB. For example a locus on chromosome Xq24 showed borderline evidence for association with PDB (rs5910578 within SLC25A43 gene; combined P=1.26×10⁻⁷; OR=1.34; Pₕₑₜ=0.44; I²=0.0%) as did another locus on chromosome 6p22.3 (rs1341239 near PRL gene; combined P=3.83×10⁻⁶; OR=1.20; Pₕₑₜ=0.63; I²=0.0%; Table 3 (Example 2)). Given that we observed 6 genotyped variants with P<1×10⁻⁵ in the GWAS stage after removal of confirmed SNPs and associated variants when we only expect 3 by chance (Supplementary FIG. 3), it is likely that some of the associations observed are true but our study was not sufficiently powered to detect them at a genome wide significance level (P<5×10⁻⁸).

This study has been successful in identifying seven loci that contribute substantially to the risk of developing PDB. The identified loci have relatively large effect sizes compared with other common diseases such as osteoporosis and rheumatoid arthritis. This indicates that susceptibility to PDB is most probably mediated by inheritance of a relatively small number of genes with large effect sizes as opposed to a large number of genes with small effect sizes as seen in other complex diseases. Many of the susceptibility variants lie within or close to genes that are known to play important roles in regulating osteoclast differentiation and function whereas other variants lie within genes not previously implicated in the regulation of bone metabolism. Whilst further work will be required to identify the functional variants, the present study has provided new insights into the genetic architecture of PDB and has identified several genes that previously were not suspected to play a role in bone metabolism. Finally, the large effect size of the variants identified means that it may be possible in the future to identify people at risk of developing PDB by genetic profiling.

### References for Example 2

1. Cooper, C. et al. The epidemiology of Paget's disease in Britain: is the prevalence decreasing? J. Bone Miner. Res. 14, 192-197 (1999).
2. Siris, E. S. Paget's disease of bone. J. Bone Miner. Res. 13, 1061-1065 (1998).
3. Morales-Piga, A. A., Rey-Rey, J. S., Corres-Gonzalez, J., Garcia-Sagredo, J. M. & Lopez-Abente, G. Frequency and characteristics of familial aggregation of Paget's disease of bone. J. Bone Miner. Res. 10, 663-670 (1995).
4. Laurin, N., Brown, J. P., Morissette, J. & Raymond, V. Recurrent mutation of the gene encoding sequestosome 1 (SQSTM1/p62) in Paget disease of bone. Am. J. Hum. Genet. 70, 1582-1588 (2002).
5. Hocking, L. J. et al. Domain-specific mutations in sequestosome 1 (SQSTM1) cause familial and sporadic Paget's disease. Hum. Mol. Genet. 11, 2735-2739 (2002).
6. Lucas, G. J. et al. Identification of a major locus for Paget's disease on chromosome 10p13 in families of British descent. J. Bone Miner. Res. 23, 58-63 (2008).
7. Hocking, L. J. et al. Genomewide search in familial Paget disease of bone shows evidence of genetic heterogeneity with candidate loci on chromosomes 2q36, 10p13, and 5q35. Am. J. Hum. Genet. 69, 1055-1061 (2001).
8. Laurin, N. et al. Paget disease of bone: mapping of two loci at 5q35-qter and 5q31. Am. J. Hum. Genet. 69, 528-543 (2001).
9. Tenesa, A. et al. Genome-wide association scan identifies a colorectal cancer susceptibility locus on 11q23 and replicates risk loci at 8q24 and 18q21. Nat. Genet.
40, 631-637 (2008).
10. Clayton, D. G. et al. Population structure, differential bias and genomic control in a large-scale, case-control association study. Nat. Genet. 37, 1243-1246 (2005).
11. Wellcome Trust Case Control Consortium. Genome-wide association study of 14,000 cases of seven common diseases and 3,000 shared controls. Nature 447, 661-678 (2007).
12. Novembre, J. et al. Genes mirror geography within Europe. Nature 456, 98-101 (2008).
13. Tsurukai, T., Udagawa, N., Matsuzaki, K., Takahashi, N. & Suda, T. Roles of macrophage-colony stimulating factor and osteoclast differentiation factor in osteoclastogenesis. J. Bone Miner. Metab. 18, 177-184 (2000).
14. Bouyer, P. et al. Colony-stimulating factor-1 increases osteoclast intracellular pH and promotes survival via the electroneutral Na/HCO3 cotransporter NBCn1. Endocrinology 148, 831-840 (2007).
15. Van Wesenbeeck, L. et al. The osteopetrotic mutation toothless OD is a loss-of-function frameshift mutation in the rat Csf1 gene: evidence of a crucial role for CSF-1 in osteoclastogenesis and endochondral ossification. Proc. Natl. Acad. Sci. USA 99, 14303-14308 (2002).
16. Wiktor-Jedrzejczak, W et al. Total absence of colony-stimulating factor 1 in the macrophage-deficient osteopetrotic (op/op) mouse. Proc. Natl. Acad. Sci. USA 87, 4828-4832 (1990).
17. Neale, S. D., Schulze, E., Smith, R. & Athanasou, N. A. The influence of serum cytokines and growth factors on osteoclast formation in Paget's disease. QJM 95, 233-240 (2002).
18. van Es, M. A. et al. Genome-wide association study identifies 19p13.3 (UNC13A) and 9p21.2 as susceptibility loci for sporadic amyotrophic lateral sclerosis. Nat. Genet. 41, 1083-1087 (2009).
19. Rioux, J. D. et al. Genome-wide association study identifies new susceptibility loci for Crohn disease and implicates autophagy in disease pathogenesis. Nat. Genet. 39, 596-604 (2007).
20. Rezaie, T. et al. Adult-onset primary open-angle glaucoma caused by mutations in optineurin. Science 295, 1077-1079 (2002).
21. Rezaie, T., Waitzman, D. M., Seeman, J. L., Kaufman, P. L. & Sarfarazi, M.
Molecular cloning and expression profiling of optineurin in the rhesus monkey. Invest. Ophthalmol. Vis. Sci. 46, 2404-2410 (2005).
22. Zhu, G., Wu, C. J., Zhao, Y. & Ashwell, J. D. Optineurin negatively regulates TNFα-induced NF-κB activation by competing with NEMO for ubiquitinated RIP. Curr. Biol. 17, 1438-1443 (2007).
23. Sudhakar, C., Nagabhushana, A., Jain, N. & Swamp, G. NF-κB mediates tumor necrosis factor α-induced expression of optineurin, a negative regulator of NF-κB. PLoS One 4, e5114 (2009).
24. Sahlender, D. A. et al. Optineurin links myosin VI to the Golgi complex and is involved in Golgi organization and exocytosis. J. Cell Biol. 169, 285-295 (2005).
25. Watts, G. D. et al. Inclusion body myopathy associated with Paget disease of bone and frontotemporal dementia is caused by mutant valosin-containing protein. Nat. Genet. 36, 377-381 (2004).
26. Li, J. et al. RANK is the intrinsic hematopoietic cell surface receptor that controls osteoclastogenesis and regulation of bone mass and calcium metabolism. Proc. Natl. Acad. Sci. USA 97, 1566-1571 (2000).
27. Villa, A., Guerrini, M. M., Cassani, B., Pangrazio, A. & Sobacchi, C. Infantile malignant, autosomal recessive osteopetrosis: the rich and the poor. Calcif. Tissue Int. 84, 1-12 (2009).
28. Hughes, A. E. et al. Mutations in TNFRSF11A, affecting the signal peptide of RANK, cause familial expansile osteolysis. Nat. Genet. 24, 45-48 (2000).
29. Nakatsuka, K., Nishizawa, Y. & Ralston, S. H. Phenotypic characterization of early onset Paget's disease of bone caused by a 27-bp duplication in the TNFRSF11A gene. J. Bone Miner. Res. 18, 1381-1385 (2003).
30. Whyte, M. P. & Hughes, A. E. Expansile skeletal hyperphosphatasia is caused by a 15-base pair tandem duplication in TNFRSF11A encoding RANK and is allelic to familial expansile osteolysis. J. Bone Miner. Res. 17, 26-29 (2002).
31. Wuyts, W. et al. Evaluation of the role of RANK and OPG genes in Paget's disease of bone. Bone 28, 104-107 (2001).
32. Hocking, L. et al. Familial Paget's disease of bone: patterns of inheritance and frequency of linkage to chromosome 18q. Bone 26, 577-580 (2000).
33. Styrkarsdottir, U. et al. Multiple genetic loci for bone mineral density and fractures. N Engl. J. Med. 358, 2355-2365 (2008).
34. Rivadeneira, F. et al. Twenty bone-mineral-density loci identified by large-scale meta-analysis of genome-wide association studies. Nat. Genet. 41, 1199-1206 (2009).
35. Styrkarsdottir, U. et al. New sequence variants associated with bone mineral density. Nat. Genet. 41, 15-17 (2009).
36. Saito, K. et al. A novel binding protein composed of homophilic tetramer exhibits unique properties for the small GTPase Rab5. J. Biol. Chem. 277, 3412-3418 (2002).
37. Barrett, J. C., Fry, B., Mailer, J. & Daly, M. J. Haploview: analysis and visualization of LD and haplotype maps. Bioinformatics 21, 263-265 (2005).
38. Langston, A. L. et al. Randomized trial of intensive bisphosphonate treatment versus symptomatic management in Paget's disease of bone. J. Bone Miner. Res. 25, 20-31 (2010).
39. Purcell, S. et al. PLINK: a tool set for whole-genome association and population-based linkage analyses. Am. J. Hum. Genet. 81, 559-575 (2007).
40. Price, A. L. et al. Long-range LD can confound genome scans in admixed populations. Am. J. Hum. Genet. 83, 132-135 (2008).
41. Li, Y. & Mach Abecasis, G. R. 1.0: Rapid haplotype reconstruction and missing genotype inference. Am. J. Hum. Genet. S79, 2290 (2006).
42. Li, Y., Willer, C., Sanna, S. & Abecasis, G. Genotype imputation. Annu. Rev. Genomics Hum. Genet. 10, 387-406 (2009).
43. Pe'er, I., Yelensky, R., Altshuler, D. & Daly, M. J. Estimation of the multiple testing burden for genomewide association studies of nearly all common variants. Genet. Epidemiol. 32, 381-385 (2008).

### Tables for Example 2 are Presented Below

TABLE 1
(Example 2). Summary of the seven loci showing genome-wide
significant association with Paget's disease of bone.

| | | | | | Replication | | Combined Overall Effect |
|---|---|---|---|---|---|---|---|
| | | | GWAS Stage | | Fixed effect | | Fixed effect |
| Chr | SNP | RA | P | OR (95% CI) | P | OR (95% CI) | P |
| 1 | rs10494112 | G | 5.83 × 10⁻¹⁷ | 1.75 (1.54-1.99) | 4.93 × 10⁻¹⁹ | 1.69 (1.50-1.89) | 7.06 × 10⁻³⁵ |
| 7 | rs4294134 | G | 1.20 × 10⁻⁰⁵ | 1.50 (1.25-1.79) | 2.29 × 10⁻⁰⁵ | 1.42 (1.20-1.66) | 8.45 × 10⁻¹⁰ |
| 8 | rs2458413 | A | 7.85 × 10⁻¹¹ | 1.51 (1.34-1.71) | 1.09 × 10⁻⁰⁷ | 1.32 (1.19-1.46) | 7.38 × 10⁻¹⁷ |
| 10 | rs1561570 | T | 9.56 × 10⁻¹⁸ | 1.71 (1.51-1.93) | 2.09 × 10⁻²¹ | 1.64 (1.48-1.81) | 4.37 × 10⁻³⁸ |
| 14 | rs10498635 | C | 1.51 × 10⁻⁰⁵ | 1.45 (1.23-1.71) | 5.64 × 10⁻⁰⁷ | 1.42 (1.29-1.63) | 2.55 × 10⁻¹¹ |
| 15 | rs5742915 | C | 1.40 × 10⁻⁰⁷ | 1.38 (1.22-1.54) | 3.99 × 10⁻⁰⁸ | 1.32 (1.20-1.46) | 1.60 × 10⁻¹⁴ |
| 18 | rs3018362 | A | 1.87 × 10⁻¹¹ | 1.50 (1.34-1.69) | 1.27 × 10⁻¹⁰ | 1.40 (1.26-1.55) | 7.98 × 10⁻²¹ |

| | Combined Overall Effect | | | | | |
|---|---|---|---|---|---|---|
| | Fixed effect | Random effects | | | | |
| Chr | OR (95% CI) | P | OR (95% CI) | Pₕₑₜ | I² | Closest Gene |
| 1 | 1.72 (1.57-1.87) | 7.06 × 10⁻³⁵ | 1.72 (1.57-1.87) | 0.97 | 00.0 | CSF1, |
| | | | | | | EPS8LS |
| 7 | 1.45 (1.29-1.63) | 8.45 × 10⁻¹⁰ | 1.45 (1.29-1.63) | 0.83 | 00.0 | NUP205 |
| 8 | 1.40 (1.29-1.51) | 1.61 × 10⁻⁰⁷ | 1.36 (1.21-1.53) | 0.10 | 44.3 | TM7SF4 |
| 10 | 1.67 (1.54-1.80) | 4.34 × 10⁻¹² | 1.68 (1.45-1.95) | 0.01 | 65.7 | OPTN |
| 14 | 1.44 (1.29-1.60) | 2.55 × 10⁻¹¹ | 1.44 (1.29-1.60) | 0.62 | 00.0 | RIN3 |
| 15 | 1.34 (1.25-1.45) | 1.60 × 10⁻¹⁴ | 1.34 (1.25-1.45) | 0.56 | 00.0 | PML |
| 18 | 1.45 (1.34-1.56) | 7.98 × 10⁻²¹ | 1.45 (1.34-1.56) | 0.46 | 00.0 | TNFRSF11A |

RA, risk allele; OR, odds ratio for the risk allele; CI, confidence interval; I², heterogeneity statistics; Pₕₑₜ, P-value for heterogeneity. Newly identified loci are shown in bold letters.

TABLE 2
(Example 2) Summary of replication study groups

| | | Mean | Males | Females |
|---|---|---|---|---|
| Sample group | Number | Age ± SD | (n) | (n) |
| Belgian cases | 246 | 73.1 ± 10.4 | 136 | 110 |
| Belgian control | 263 | 71.2 ± 7.0 | 138 | 125 |
| Dutch cases | 85 | 69.8 ± 9.5 | 46 | 39 |
| Dutch controls | 93 | 67.1 ± 6.5 | 47 | 46 |
| Italian Replication 1 cases | 354 | 69.3 ± 9.8 | 195 | 159 |
| Italian Replication 1 controls | 390 | 60.9 ± 12.2 | 215 | 175 |
| Italian Replication 2 cases | 205 | 71.7 ± 11.4 | 118 | 87 |
| Italian Replication 2 controls | 238 | 66.5 ± 6.8 | 122 | 116 |
| Spanish cases | 186 | 68.2 ± 11.5 | 110 | 76 |
| Spanish controls | 202 | 74.6 ± 9.4 | 116 | 86 |
| UK/Australian cases | 398 | 79.6 ± 9.2 | 207 | 191 |
| UK/Australian controls | 485 | 71.0 ± 10.42 | 249 | 236 |

Details of the number, age, and gender of cases and controls from each of the replication cohorts is shown. The distribution of males and females was not statistically different between cases and controls in any of the cohorts studied (P > 0.18).

TABLE 3
(Example 2). Association test results of 27 SNPs genotyped in the replication samples.

| | | | | GWAS | | | | Replication | |
|---|---|---|---|---|---|---|---|---|---|
| | | | | RAF | | | | Fixed effect | |
| CHR | SNP | Position | RA | Cases | Controls | P | OR | P | OR |
| 1 | rs11578700 | 105,093,642 | A | 0.800 | 0.748 | 5.26 × 10⁻⁰⁵ | 1.35 | 4.12 × 10⁻⁰¹ | 1.05 |
| 1 | rs10494112 | 110,154,000 | G | 0.308 | 0.203 | 5.83 × 10⁻¹⁷ | 1.75 | 4.93 × 10⁻¹⁹ | 1.69 |
| 1 | rs484959 | 110,167,606 | G | 0.639 | 0.527 | 6.48 × 10⁻¹⁴ | 1.59 | 1.24 × 10⁻²¹ | 1.67 |
| 1 | rs10926978 | 241,498,277 | T | 0.730 | 0.674 | 5.08 × 10⁻⁰⁵ | 1.31 | 1.77 × 10⁻⁰¹ | 1.07 |
| 3 | rs9841941 | 82,971,603 | A | 0.649 | 0.585 | 2.24 × 10⁻⁰⁵ | 1.31 | 6.74 × 10⁻⁰¹ | 1.02 |
| 4 | rs1871584 | 5,796,621 | C | 0.568 | 0.504 | 2.16 × 10⁻⁰⁵ | 1.29 | 2.55 × 10⁻⁰¹ | 0.94 |
| 4 | rs2973276 | 37,273,986 | T | 0.781 | 0.726 | 4.26 × 10⁻⁰⁵ | 1.34 | 1.53 × 10⁻⁰¹ | 0.92 |
| 6 | rs1341239 | 22,412,183 | T | 0.437 | 0.377 | 4.20 × 10⁻⁰⁵ | 1.29 | 1.35 × 10⁻⁰² | 1.14 |
| 7 | rs7801223 | 93,032,526 | A | 0.147 | 0.106 | 2.51 × 10⁻⁰⁵ | 1.45 | 6.49 × 10⁻⁰¹ | 1.04 |
| 7 | rs4294134 | 134,943,668 | G | 0.886 | 0.839 | 1.20 × 10⁻⁰⁵ | 1.50 | 2.29 × 10⁻⁰⁵ | 1.42 |
| 8 | rs1545370 | 105,244,117 | G | 0.389 | 0.319 | 9.25 × 10⁻⁰⁷ | 1.36 | 1.05 × 10⁻⁰² | 1.15 |
| 8 | rs2458413 | 105,428,608 | A | 0.674 | 0.577 | 7.85 × 10⁻¹¹ | 1.51 | 1.09 × 10⁻⁰⁷ | 1.32 |
| 8 | rs2669429 | 105,532,866 | T | 0.505 | 0.436 | 4.07 × 10⁻⁰⁶ | 1.32 | 1.07 × 10⁻⁰² | 1.14 |
| 9 | rs892688 | 26,229,410 | T | 0.095 | 0.062 | 2.19 × 10⁻⁰⁵ | 1.58 | 1.53 × 10⁻⁰¹ | 0.86 |
| 10 | rs1561570 | 13,195,732 | T | 0.659 | 0.531 | 9.56 × 10⁻¹⁸ | 1.71 | 2.09 × 10⁻²¹ | 1.64 |
| 10 | rs825411 | 13,209,380 | C | 0.607 | 0.531 | 3.22 × 10⁻⁰⁷ | 1.37 | 2.33 × 10⁻⁰⁶ | 1.27 |
| 10 | rs10994443 | 62,065,524 | G | 0.944 | 0.892 | 3.92 × 10⁻⁰⁹ | 2.06 | 1.87 × 10⁻⁰¹ | 0.89 |
| 14 | rs10498635 | 92,173,062 | C | 0.865 | 0.816 | 1.51 × 10⁻⁰⁵ | 1.45 | 5.64 × 10⁻⁰⁷ | 1.42 |
| 15 | rs5742915 | 72,123,686 | C | 0.530 | 0.451 | 1.40 × 10⁻⁰⁷ | 1.38 | 3.99 × 10⁻⁰⁸ | 1.32 |
| 16 | rs1510189 | 54,658,025 | A | 0.563 | 0.492 | 2.76 × 10⁻⁰⁶ | 1.33 | 8.50 × 10⁻⁰² | 1.09 |
| 17 | rs2304961 | 908,650 | G | 0.394 | 0.331 | 9.14 × 10⁻⁰⁶ | 1.32 | 5.06 × 10⁻⁰¹ | 0.96 |
| 18 | rs1551821 | 41,120,468 | T | 0.888 | 0.828 | 6.17 × 10⁻⁰⁸ | 1.65 | 1.15 × 10⁻⁰¹ | 1.11 |
| 18 | rs2957128 | 58,211,715 | A | 0.497 | 0.412 | 1.61 × 10⁻⁰⁸ | 1.41 | 1.12 × 10⁻¹⁰ | 1.39 |
| 18 | rs3018362 | 58,233,073 | A | 0.455 | 0.357 | 1.87 × 10⁻¹¹ | 1.50 | 1.27 × 10⁻¹⁰ | 1.40 |
| 19 | rs265558 | 17,755,878 | T | 0.429 | 0.368 | 3.56 × 10⁻⁰⁵ | 1.29 | 9.41 × 10⁻⁰¹ | 1.00 |
| X | rs760871 | 96,164,943 | C | 0.024 | 0.007 | 5.04 × 10⁻⁰⁶ | 3.36 | 7.80 × 10⁻⁰¹ | 0.94 |
| X | rs5910578 | 118,451,730 | C | 0.810 | 0.744 | 1.15 × 10⁻⁰⁵ | 1.47 | 1.69 × 10⁻⁰³ | 1.25 |

| | Combined Overall Effect | | | | | |
|---|---|---|---|---|---|---|
| | Fixed effect | | Random effects | | | |
| CHR | P | OR | P | OR | Pₕₑₜ | I² |
| 1 | 1.36 × 10⁻⁰³ | 1.15 | 3.73 × 10⁻⁰² | 1.12 | 0.21 | 28.2 |
| 1 | 7.06 × 10⁻³⁵ | 1.72 | 7.06 × 10⁻³⁵ | 1.72 | 0.97 | 00.0 |
| 1 | 2.14 × 10⁻³⁴ | 1.63 | 3.57 × 10⁻²⁸ | 1.63 | 0.34 | 11.4 |
| 1 | 2.44 × 10⁻⁰⁴ | 1.16 | 3.57 × 10⁻⁰³ | 1.15 | 0.31 | 15.5 |
| 3 | 1.91 × 10⁻⁰³ | 1.13 | 2.23 × 10⁻⁰¹ | 1.09 | 0.02 | 59.7 |
| 4 | 4.94 × 10⁻⁰² | 1.08 | 7.45 × 10⁻⁰¹ | 0.97 | 8.0 × 10⁻⁴ | 74.1 |
| 4 | 1.27 × 10⁻⁰¹ | 1.07 | 7.04 × 10⁻⁰¹ | 0.96 | 3.0 × 10⁻⁴ | 76.1 |
| 6 | 3.83 × 10⁻⁰⁶ | 1.20 | 3.83 × 10⁻⁰⁶ | 1.20 | 0.63 | 00.0 |
| 7 | 1.18 × 10⁻⁰³ | 1.21 | 1.54 × 10⁻⁰¹ | 1.13 | 0.11 | 42.2 |
| 7 | 8.45 × 10⁻¹⁰ | 1.45 | 8.45 × 10⁻¹⁰ | 1.45 | 0.83 | 00.0 |
| 8 | 1.76 × 10⁻⁰⁷ | 1.23 | 1.47 × 10⁻⁰² | 1.18 | 0.03 | 56.4 |
| 8 | 7.38 × 10⁻¹⁷ | 1.40 | 1.61 × 10⁻⁰⁷ | 1.36 | 0.10 | 44.3 |
| 8 | 5.38 × 10⁻⁰⁷ | 1.21 | 1.79 × 10⁻⁰³ | 1.19 | 0.13 | 39.9 |
| 9 | 4.38 × 10⁻⁰² | 1.16 | 7.31 × 10⁻⁰¹ | 0.95 | 1.6 × 10⁻³ | 71.8 |
| 10 | 4.37 × 10⁻³⁸ | 1.67 | 4.34 × 10⁻¹² | 1.68 | 0.01 | 65.7 |
| 10 | 2.79 × 10⁻¹² | 1.31 | 1.39 × 10⁻¹⁰ | 1.31 | 0.37 | 08.0 |
| 10 | 2.10 × 10⁻⁰² | 1.17 | 8.40 × 10⁻⁰¹ | 1.04 | 1.0 × 10⁻⁵ | 01.6 |
| 14 | 2.55 × 10⁻¹¹ | 1.44 | 2.55 × 10⁻¹¹ | 1.44 | 0.62 | 00.0 |
| 15 | 1.60 × 10⁻¹⁴ | 1.34 | 1.60 × 10⁻¹⁴ | 1.34 | 0.56 | 00.0 |
| 16 | 1.01 × 10⁻⁰⁵ | 1.19 | 1.90 × 10⁻⁰² | 1.14 | 0.10 | 44.2 |
| 17 | 1.17 × 10⁻⁰² | 1.11 | 7.49 × 10⁻⁰¹ | 1.02 | 0.01 | 62.7 |
| 18 | 6.75 × 10⁻⁰⁶ | 1.27 | 2.80 × 10⁻⁰² | 1.21 | 0.02 | 58.5 |
| 18 | 4.73 × 10⁻¹⁸ | 1.40 | 3.44 × 10⁻⁰⁸ | 1.41 | 0.05 | 51.7 |
| 18 | 7.98 × 10⁻²¹ | 1.45 | 7.98 × 10⁻²¹ | 1.45 | 0.46 | 00.0 |
| 19 | 4.74 × 10⁻⁰³ | 1.12 | 2.83 × 10⁻⁰¹ | 1.07 | 0.07 | 48.1 |
| X | 8.08 × 10⁻⁰³ | 1.60 | 7.45 × 10⁻⁰¹ | 1.15 | 4.0 × 10⁻⁴ | 75.9 |
| X | 1.26 × 10⁻⁰⁷ | 1.34 | 1.26 × 10⁻⁰⁷ | 1.34 | 0.44 | 00.0 |

RA, risk allele; RAF, risk allele frequency; OR, odds ratio for the risk allele; CI, confidence interval; I², Heterogeneity statistic; Pₕₑₜ, P-value for heterogeneity.

TABLE 4
(Example 2). Details of SNPs showing genome-wide significant
association with Paget's disease of bone

| | | | | Study Group | | |
|---|---|---|---|---|---|---|
| Locus | SNP | Gene | RA | Name | Cases | Controls |
| 7q33 | rs4294134 | NUP205 | G | GWAS | 741 | 2,699 |
| | | | | UK/Australia Replication | 398 | 485 |
| | | | | Italian Replication 1 | 354 | 390 |
| | | | | Belgian Replication | 246 | 263 |
| | | | | Italian Replication 2 | 205 | 238 |
| | | | | Spanish Replication | 186 | 202 |
| | | | | Dutch Replication | 85 | 93 |
| | | | | Overall Fixed effect | 2,215 | 4,370 |
| | | | | Overall Random effects | 2,215 | 4,370 |
| 8q22.3 | rs2458413 | TM7SF4 | A | GWAS | 741 | 2,699 |
| | | | | UK/Australia Replication | 398 | 485 |
| | | | | Italian Replication 1 | 354 | 390 |
| | | | | Belgian Replication | 246 | 263 |
| | | | | Italian Replication 2 | 205 | 238 |
| | | | | Spanish Replication | 186 | 202 |
| | | | | Dutch Replication | 85 | 93 |
| | | | | Overall Fixed effect | 2,215 | 4,370 |
| | | | | Overall Random effects | 2,215 | 4,370 |
| 14q32.12 | rs10498635 | RIN3 | C | GWAS | 741 | 2,699 |
| | | | | UK/Australia Replication | 398 | 485 |
| | | | | Italian Replication 1 | 354 | 390 |
| | | | | Belgian Replication | 246 | 263 |
| | | | | Italian Replication 2 | 205 | 238 |
| | | | | Spanish Replication | 186 | 202 |
| | | | | Dutch Replication | 85 | 93 |
| | | | | Overall Fixed effect | 2,215 | 4,370 |
| | | | | Overall Random effects | 2,215 | 4,370 |
| 15q24.1 | rs5742915 | PML | C | GWAS | 741 | 2,699 |
| | | | | UK/Australia Replication | 398 | 485 |
| | | | | Italian Replication 1 | 354 | 390 |
| | | | | Belgian Replication | 246 | 263 |
| | | | | Italian Replication 2 | 205 | 238 |
| | | | | Spanish Replication | 186 | 202 |
| | | | | Dutch Replication | 85 | 93 |
| | | | | Overall Fixed effect | 2,215 | 4,370 |
| | | | | Overall Random effects | 2,215 | 4,370 |

| | Risk Allele Frequency | | | | | | |
|---|---|---|---|---|---|---|---|
| Locus | Cases | Controls | OR | 95% CI | P | I² | Pₕₑₜ |
| 7q33 | 0.886 | 0.838 | 1.50 | 1.25-1.79 | 1.20 × 10⁻⁵ | | |
| | 0.875 | 0.849 | 1.25 | 0.95-1.65 | 0.117 | | |
| | 0.921 | 0.884 | 1.53 | 1.07-2.18 | 0.018 | | |
| | 0.874 | 0.825 | 1.47 | 1.04-2.08 | 0.030 | | |
| | 0.951 | 0.911 | 1.91 | 1.08-3.36 | 0.023 | | |
| | 0.923 | 0.888 | 1.53 | 0.92-2.52 | 0.096 | | |
| | 0.881 | 0.867 | 1.13 | 0.60-2.13 | 0.693 | | |
| | NA | NA | 1.45 | 1.29-1.63 | 8.45 × 10⁻¹⁰ | | |
| | NA | NA | 1.45 | 1.29-1.63 | 8.45 × 10⁻¹⁰ | 00.0 | 0.83 |
| 8q22.3 | 0.673 | 0.577 | 1.51 | 1.34-1.71 | 7.85 × 10⁻¹¹ | | |
| | 0.643 | 0.580 | 1.30 | 1.07-1.58 | 0.007 | | |
| | 0.639 | 0.592 | 1.22 | 0.99-1.51 | 0.065 | | |
| | 0.689 | 0.572 | 1.66 | 1.28-2.14 | 1.16 × 10⁻⁴ | | |
| | 0.669 | 0.576 | 1.49 | 1.13-1.96 | 0.004 | | |
| | 0.626 | 0.572 | 1.25 | 0.94-1.67 | 0.124 | | |
| | 0.606 | 0.638 | 0.87 | 0.57-1.34 | 0.527 | | |
| | NA | NA | 1.40 | 1.29-1.51 | 7.38 × 10⁻¹⁷ | | |
| | NA | NA | 1.36 | 1.21-1.53 | 1.61 × 10⁻⁷ | 44.3 | 0.10 |
| 14q32.12 | 0.865 | 0.815 | 1.45 | 1.23-1.71 | 1.51 × 10⁻⁵ | | |
| | 0.869 | 0.817 | 1.48 | 1.12-1.93 | 0.003 | | |
| | 0.856 | 0.814 | 1.36 | 1.03-1.79 | 0.029 | | |
| | 0.896 | 0.829 | 1.78 | 1.23-2.57 | 0.002 | | |
| | 0.841 | 0.809 | 1.25 | 0.88-1.78 | 0.203 | | |
| | 0.857 | 0.842 | 1.13 | 0.76-1.68 | 0.535 | | |
| | 0.887 | 0.801 | 1.95 | 1.07-3.54 | 0.027 | | |
| | NA | NA | 1.44 | 1.29-1.60 | 2.55 × 10⁻¹¹ | | |
| | NA | NA | 1.44 | 1.29-1.60 | 2.55 × 10⁻¹¹ | 00.0 | 0.62 |
| 15q24.1 | 0.530 | 0.451 | 1.38 | 1.22-1.54 | 1.40 × 10⁻⁷ | | |
| | 0.531 | 0.468 | 1.29 | 1.07-1.55 | 0.008 | | |
| | 0.566 | 0.482 | 1.40 | 1.14-1.72 | 0.001 | | |
| | 0.474 | 0.449 | 1.10 | 0.86-1.41 | 0.426 | | |
| | 0.544 | 0.485 | 1.26 | 0.97-1.65 | 0.081 | | |
| | 0.549 | 0.453 | 1.47 | 1.11-1.95 | 0.0078 | | |
| | 0.518 | 0.383 | 1.73 | 1.13-2.63 | 0.010 | | |
| | NA | NA | 1.34 | 1.25-1.45 | 1.60 × 10⁻¹⁴ | | |
| | NA | NA | 1.34 | 1.25-1.45 | 1.60 × 10⁻¹⁴ | 00.0 | 0.56 |

RA, risk allele; OR, odds ratio for the risk allele; CI, confidence interval; I², Heterogeneity statistic; Pₕₑₜ, F-value for heterogeneity.

TABLE 5
(Example 2) Cross-Platform Genotype Concordance Rate¹

| | | Samples | SNPs | Genotype |
|---|---|---|---|---|
| | | Genotyped on | Genotyped on | Concordance |
| | | Both Platforms | Both Platforms | Rate Between |
| Platform 1 | Platform 2 | (n) | (n) | Platforms (%) |
| HumanHap300-Duo | Sequenom iPLEX | 96 | 27 | 99.93 |
| Human660W-Quad | Sequenom iPLEX | 10 | 27 | 99.76 |
| HumanHap300-Duo | Human660W-Quad | 6 | 290,115 | 99.80 |
| Human1.2M-Duo | Affymetrix v 6.0 | 2,779 | 88,146 | 99.79 |

¹As a quality control measure, a subset of PDB samples used in the GWAS stage were genotyped on two different platforms and genotype concordance rate was calculated. For control samples which were genotyped by WTCCC using illumina Human 1.2M-Duo chip, the same samples were also genotyped using Affymetrix v6.0 and genotype concordance rate was calculated for the 88,146 overlapping SNPs (out of 290,115 SNPs used in GWAS analysis). Only genotypes from illumina Human 1.2M chip were used for GWAS analysis.

TABLE 6
(Example 2) Genotype call rate for top hits associated with PDB risk

| | | GWAS | | Replication | |
|---|---|---|---|---|---|
| | | samples Genotype | | samples Genotype | |
| | | Call Rate (%) | | Call Rate (%) | |
| | | Cases | Controls | Cases | Controls |
| Chr | SNP | (n = 741) | (n = 2699) | (n = 1474) | (n = 1671) |
| 1 | rs10494112 | 98.92 | 99.96 | 99.93 | 100.00 |
| 7 | rs4294134 | 98.25 | 99.89 | 98.03 | 95.57 |
| 8 | rs2458413 | 99.19 | 99.96 | 99.25 | 99.88 |
| 10 | rs1561570 | 99.73 | 99.96 | 99.86 | 99.52 |
| 14 | rs10498635 | 98.11 | 99.59 | 99.86 | 99.76 |
| 15 | rs5742915 | 98.92 | 99.93 | 99.93 | 100.00 |
| 18 | rs3018362 | 100.00 | 99.93 | 99.86 | 99.88 |

1-15. (canceled)

16. A method of diagnosing Paget's disease of bone (PDB) or identifying an altered risk of developing PDB in a subject, said method comprising the steps of probing a sample provided by said subject, for sequence variations at or within one or more of the chromosomal loci selected from the group consisting of:
(i) 10p13;
(ii) 1p13.3;
(iii) 18q21;
(iv) 8q22.3;
(v) 7q33;
(vi) 14q32.12;
(vii) 15q24.1;
(viii) 6p22.3; and
(ix) Xq24;
wherein sequence variants are detected by comparison with a reference sequence and subjects identified as harbouring sequence variations at or within one or more of the chromosomal loci (i)-(ix), may have Paget's disease of bone (PDB) or may be at an altered risk of developing PDB.

17. The method of claim 16, wherein the sample is probed using an antibody having affinity for or selective/specific for, a polypeptide or an epitope thereof encoded by the variant sequence.

18. The method of claim 16, wherein the sample is probed using oligonucleotide primers which amplify the variant nucleic acid sequence or an oligonucleotide probe which hybridises to the variant nucleic acid sequence.

19. The method of claim 16, wherein the sample provided by the subject is probed for sequence variations within the 10p13 locus.

20. The method of claim 19, wherein the sample is probed using an antibody having affinity for or selective/specific for, a polypeptide or an epitope thereof encoded by a variant sequence at or within the chromosomal loci 10p13.

21. The method of claim 19, wherein the sample is probed using oligonucleotide primers which amplify a variant nucleic acid sequence at or within the chromosomal locus 10p13 or an oligonucleotide probe which hybrises to a variant nucleic acid sequence at or within the chromosomal locus 10p13.

22. The method of claim 19, wherein the sample is probed for the presence of a variant OPTN sequence.

23. The method of claim 19, wherein the sample is probed for a variant OPTN gene, wherein the variant OPTN sequence comprises the rs1561570 SNP.

24. The method of claim 19, wherein the sample is probed using an antibody having affinity for or selective/specific for, a polypeptide or an epitope thereof encoded by a variant OPTN sequence.

25. The method of claim 19, wherein the sample is probed using oligonucleotide primers which amplify a variant OPTN nucleic acid sequence or an oligonucleotide probe which hybrises to a variant OPTN nucleic acid sequence.

26. The method of claim 19, wherein the sample provided by the subject is further probed for sequence variations within one or more of the chromosomal loci selected from the group consisting of:
(i) 1p13.3;
(ii) 18q21;
(iii) 8q22.3;
(iv) 7q33;
(v) 14q32.12;
(vi) 15q24.1;
(vii) 6p22.3; and
(viii) Xq24.

27. The method of claim 16, wherein the sample is probed for sequence variations within or near/adjacent to, one or more of the genes selected from the group consisting of:
(i) OPTN;
(ii) CSF1;
(iii) TNFRSF11A;
(iv) TM7SF4;
(v) RIMS2;
(vi) DPYS;
(vii) CNOT4;
(viii) NUP205;
(ix) SLC13A4;
(x) RIN3;
(xi) PML;
(xii) PRL and
(xiii) SLC25A43

28. The method of claim 27, wherein the sample is probed for a sequence variation within or near/adjacent to the OPTN gene and/or for the presence of the rs1561570 SNP.

29. The method of claim 16, wherein the method comprises probing the sample for the presence of one or more SNPs selected from the group consisting of:
(i) rs1561570
(ii) rs10494112;
(iii) rs499345;
(iv) rs484959;
(v) rs2458413;
(vi) rs2957128;
(vii) rs3018362;
(viii) rs4294134;
(ix) rs10498635;
(x) rs5742915;
(xi) rs1341239;
(xii) rs5910578; and
(xiii) SNPs or sequence variations in linkage disequilibrium with any of (i)-(xii).

30. The method of claim 29, wherein the sample is probed for the presence of the rs1561570 SNP.

31. The method of claim 30, wherein the sample is further probed for one or more of the SNPs selected from the group consisting of:
(i) rs10494112;
(ii) rs499345;
(iii) rs484959;
(iv) rs2458413;
(v) rs2957128;
(vi) rs3018362;
(vii) rs4294134;
(viii) rs10498635;
(ix) rs5742915;
(x) rs1341239
(xi) rs5910578; and
(xii) SNPs or sequence variations in linkage disequilibrium with any of (i)-(xii).

32. The method claim 28, wherein the method further comprises probing the sample for variant SQSTM1 sequences.

33. The method of claim 29, wherein the method of determining a subject's altered risk of developing PDB comprises identifying the number of PDB risk alleles present at one or more of the SNP locations and calculating a risk allele score, wherein the risk allele score is indicative of the subject's altered risk of developing PDB.

34. A method of screening patients for PDB and/or a predisposition/susceptibility thereto, said method comprising the step of subjecting a sample provided by a subject to be tested to the method of claim 16, wherein subjects identified as having provided a sample indicating PDB or an altered risk of developing PDB, are recommended for treatment with a medicament or composition to treat and/or prevent PDB.

35. The method of claim 16, wherein the sample provided by the subject to be tested is probed using molecular or PCR based and/or immunological techniques.

36. A method of treating PDB, said method comprising administering a therapeutically effective amount of one or more of the genes or polynucleotides selected from the group consisting of:
(i) OPTN;
(ii) CSF1;
(iii) TNFRSF11A;
(iv) TM7SF4;
(v) RIMS2;
(vi) DPYS;
(vii) CNOT4;
(viii) NUP205;
(ix) SLC13A4;
(x) RIN3;
(xi) PML;
(xii) PRL;
(xiii) SLC25A4; and
(xiv) a polynucleotide fragment of any (i)-(xiii).

37. A method of treating or preventing PDB, said method comprising the step of administering a therapeutically effective amount of an antisense sequence, wherein said antisense sequences modulate the expression of one or more of the genes selected from the group consisting of:
(i) OPTN;
(ii) CSF1;
(iii) TNFRSF11A;
(iv) TM7SF4;
(v) RIMS2;
(vi) DPYS;
(vii) CNOT4;
(viii) NUP205;
(ix) SLC13A4;
(x) RIN3;
(xi) PML;
(xii) PRL; and
(xiii) SLC25A4.

38. A kit for identifying a variant 1p13.3, 10p13, 18q21, 8q22.3, 7q33, 14q32.12, 15q24.1, 6p22.3 and/or Xq24 sequence in a sample, said kit comprising pairs of oligonucleotide primers for amplifying a variant nucleotide sequence at or within one or more the chromosomal loci selected from the group consisting of:
(i) 1p13.3;
(ii) 10p13;
(iii) 18q21;
(iv) 8q22.3;
(v) 7q33;
(vi) 14q32.12;
(vii) 15q24.1;
(viii) 6p22.3; and
(ix) Xq24;

39. The kit of claim 38, wherein the kit may further comprise one or more additional components selected from the group consisting of:
(i) a polymerizing agent,
(ii) elongating nucleotides or terminating nucleotides and
(iii) reaction and/or storage buffers.
